(19) United States
(12) Patent Application Publication
(10) Pub. No.: US 2002/0177125 A1
(43) Pub. Date: Nov. 28, 2002

(54) Human rhinovirus assays, and compositions therefrom

(76) Inventors: Carl Alexander Kamb, Salt Lake City, UT (US); Mark Aaron Poritz, Salt Lake City, UT (US); David Heng-Fai Teng, Salt Lake City, UT (US)

Correspondence Address: Joseph A. Williams, Jr. Ph.D., Marshall, Gerstein, & Borun, 6300 Sears Tower, 233 South Wacker Drive, Chicago, IL, 60606-6402, US

(21) Appl. No.: 09/991,003

(22) Filed: Nov. 16, 2001

Related U.S. Application Data
(63) Continuation-in-part of application No. 08/812,994, filed on Mar. 4, 1997, now Pat. No. 5,955,275.
(63) Continuation-in-part of application No. 09/259,155, filed on Feb. 26, 1999, now abandoned.
(60) Provisional application No. 60/253,333, filed on Nov. 27, 2000.
(60) Provisional application No. 60/272,026, filed on Feb. 28, 2001.

(51) Int. Cl. C12Q 1/70; C12N 9/50; C12N 7/00; C12N 5/08; C07H 21/04

(52) U.S. Cl. 435/5; 435/69.3; 435/219; 435/235.1; 435/366; 435/320.1; 536/23.72

(57) ABSTRACT

Methods for assaying for viral-related activity are disclosed. The assays of the invention provide for the identification of biologically active phenotypic probes and cellular targets and fragments, variants and mimetics thereof.

This application claims priority from and is a continuation-in-part of Ser. No. of 08/812,994, now issued as U.S. Pat. No. 5,955,275, U.S. application Ser. No. 09/259,155, U.S. application Ser. No. 60/253,333 (VEN008/00/P1, filed Nov. 27, 2000) and U.S. application Ser. No. 60/272,026 (VEN008/00/P2, filed Feb. 28, 2001), the entire disclosures of which are specifically incorporated by reference herein in their entireties.

### BACKGROUND OF THE INVENTION

Rhinoviral pathogens are the primary agent(s) responsible for the common cold (Makela M. J. et al (1998) “Viruses and bacteria in the etiology of the common cold” Journal of Clinical Microbiology 36:539-42, Dick, E. C. et al. (1992) “Rhinoviruses” in Textbook of pediatric infectious diseases. 3^{rd} ed. Philadelphia: W B Saunders). The 100⁺ serologically distinct agents that constitute the rhinoviral family contribute prominently to the nation's total medical costs and make up a significant percentage of the nationwide employee absenteeism rate (>twenty-five million days of missed work each year in the USA). In addition, patients exhibiting the symptoms of rhinoviral infection are predisposed to secondary pathogens (e.g. bacterial) that lead to more threatening symptomologies including sinusitis, otitus media, bronchitis, pneumonia, and asthmatic exacerbation (Pitkaranta, A. et al. (1998) “Detection of rhino-virus, respiratory syncytial virus, and coronavirus infections in acute otitis media by reverse transcriptase polymerase chain reaction.” Pediatrics 102:291-295)

The clinical features of the common cold are familiar to all and result from the introduction of rhinovirus into the ciliated epithelial cells of the upper respiratory tract (Noah, T. L. et al. (1995) “Nasal cytokine production in viral acute upper respiratory infection in children.” J. Infec Dis. 171:584-592). In response to viral attack, the host releases inflammatory mediators (cytokines) such as IL-1B, TNF-α, and IL-8 as well as vasoactive agents (e.g. bradykinin) that attract inflammatory leukocytes (e.g. granulocytes and monocytes, see, for example, Pitkaranta, A. (1998) “What's new with common colds? Pathogenesis and diagnosis.” Infectious Med. 15:50-59). Thus, in rhinoviral infections, many of the symptoms are “host-induced” and it is for this reason that the common cold is often referred to as a “cytokine disease”.

Rhinoviruses, along with hepatoviruses (e.g.Hepatitis A) enteroviruses (e.g. coxsackievirus, echoviruses, enterovirses 68-71, Foot and Mouth Disease virus (FMDV)), and cardiovirus (e.g. encephalomyocarditis virus (EMC)) belong to the class of viruses known as picomaviridae (Reuckert, R. R. (1996) “Picomaviridae: The viruses and their replication.” in “Fields Virology” Lippincott-Raven Publishers, Philadelphia). The picornaviridae are all small (˜30 nm in diameter), non-enveloped viruses that carry a single (+) stranded RNA encapsulated in a protein shell with icosahedral symmetry. The rhinovirus genome consists of a single “plus” (“messenger active”) strand of RNA containing ˜7,209 base pairs (see FIG. 1) that is polyadenylated on the 3′ end and covalently linked to a small viral protein (VPg) at the 5′ end. The outer shell or capsid of the picomavirus is composed of four viral proteins (VP1-VP4) organized into subunits called protomers, with a single capsid containing sixty protomers arranged as twelve pentameric units. VP1, VP2, and VP3 are exposed to the viral surface. In contrast, VP4 lies buried below the surface of the pathogens proteinaceous exterior in close association with the RNA core (Lund G. A. et al (1977) “Distribution of capsid polypeptides with respect to the surface of the virus particle.” Virology 78:35-44). Together these proteins (i) protect the viral genome from nucleases, (ii) determine the host range or tropism of the virus, (iii) carry information for packaging the viral genome, and (iv) are responsible for delivery of the viral RNA to the cytosol of susceptible cells.

Similarities in the genome organization of various different members of the picomavirus class (e.g. rhinovirus and poliovirus) allow generalizations about the picomaviral lifecycle. Initially, viruses of this class attach themselves to the surface of the cell membrane through association with a host receptor (FIG. 2). In the case of rhinoviridae, two unique receptor classes exist. The major class of rhinoviruses (>eighty serotypes) associate with the host encoded ICAM-1 molecule (see, for example, Greve, J. M. et al. (1989) “The Major Human Rhinovirus Receptor is ICAM-1.” Cell 56:839-847) while the minor class are believed to be associated with the LDL receptor (LDLR, see, for example, Hoefer, F. et al. (1994) “Members of the low density lipoprotein receptor family mediate cell entry of a minor-group common cold virus.” PNAS 91:1839-1842). ICAM-1 is a member of the Ig superfamily and is structurally related to receptors for poliovirus, coxsackie B virus, and echoviruses. Antibodies directed against ICAM-1 protect cells from infection by rhinoviruses (see Greve et al.). Furthermore, X-ray crystallography has elucidated that the interaction between ICAM-1 and the rhinoviral surface is mediated through a cleft or “canyon” on the viral surface and a site on the ICAM-1 receptor that is distal from the plasma membrane (see, for example, Kolatkar, P. R. et al. (1999) “Structural studies of two rhinovirus serotypes complexed with fragments of their cellular receptor:” EMBO J. 18:6249-6259).

The means by which the ICAM-RV interaction leads to injection of the viral genome is poorly understood. Upon initial contact, a natural (yet unidentified) “pocket molecule” located in the viral canyon is displaced, allowing optimal shape and charge complementarity between the virus and host receptor (see for example, Oliveira, M. A. et al. (1993) “The structure of human rhinovirus 16.” Structure 1:51-68). Thereafter, in a process referred to as “eclipse”, the VP4 subunit of the viral capsid is ejected, leading to a conformational change of the protomer cassette (“uncoating”) and subsequent introduction of the viral genome into the host cell (see for example, Chow, M. Et al. (1987) Myristylation of picornavirus capsid protein VP4 and its structural significance.” Nature 327:482-486; Shepard D A et al (1993) “WIN 52035-2 inhibits both attachment and eclipse of human rhinovirus 14.” J Virology 67(4): 2245-54). Viral RNA may be injected into the host cell through a channel or pore made up of hydrophobic residues of capsid proteins (similar to the action of hemagglutinin of influenza virus, see “Fields Virology”). Alternatively, association of the virus with ICAM-1 may induce receptor-mediated endocytosis (see, for example, Madshus, I. H. (1984) “Mechanism of entry into the cytosol of poliovirus type 1: requirement for low pH.” J Cell Biol. 98:1194-1200).

Upon entry of the viral genome into the host cytosol, the virus utilizes the necessary host machinery to synthesize infectious viral particles. To accomplish this, a single long polyprotein encoding such important viral functions as (i) viral RNA initiation and elongation, (ii) capsid formation, and (iii) polyprotein processing, is translated from the viral RNA and cleaved into functional gene products by viral encoded proteases (see FIG. 2). Viral RNA that is complementary to the viral genome (i.e. the “minus” strand) is then synthesized and used as a template to expand the number of “plus” strands. Subsequently, infectious virions consisting of full-length viral genomic RNA and capsid proteins are constructed via a pathway that involves assembly and maturation of the virus. This process includes, but is not limited to such complex and poorly understood processes as, (i) threading the viral RNA molecule through an existing pore, created in an empty, immature, capsid shell (see, for example, Jacobson, M. et al. (1968) “Morphogenesis of poliovirus I. Association of the viral RNA with the coat protein.” J. Mol Biol. 33: 369-378) and (ii) converting non-virulent, immature “provirions” into infectious particles by cleavage of capsid protein VP0 into VP2 and VP4 (see, for example, Lee W. M. et al. (1993) “Role of maturation cleavage in infectivity of picornaviruses: activation of an infectosome.” J. Virology 67: 2110-2122).

Concomitant with picomaviral replication, host cellular functions are crippled to provide for optimal viral growth. For instance, the rate of host RNA synthesis declines rapidly after viral infection, due, in part, to recognition of the host polymerase and various other transcription factors by viral protease 3C (see, for example, Clark, M. E. et al. (1991) “Poliovirus proteinase 3C converts an active form of transcription factor IIIC to an inactive form; a mechanism for inhibition of host cell polymerase III transcription by poliovirus.” EMBO J 10:2941-2947; Rubenstein S. J. et al. (1992) “Infection of HeLa cells with poliovirus results in modification of a complex that binds to the rRNA promoter.” J Virology 66:3062-3068). Similarly, virally infected cells exhibit reduced levels of cellular protein synthesis; a phenomena that is most likely achieved through viral-induced cleavage of the host-encoded p220 molecule (see, for example, Etchison, D. et al. (1982) “Inhibition of HeLa cell protein synthesis following poliovirus infection correlates with the proteolysis of a 220,000-dalton polypeptide associated with eucaryotic initiation factor 3 and a cap binding protein complex.” J Biol Chem 257:14806-14810). The p220 protein is the largest subunit of the CAP binding complex (CBC) which, in turn, is responsible for attachment of the m⁷G cap group to the 5′ terminus of most cellular mRNA's. Host mRNA's require the 5′ m⁷G cap for efficient translation. Picornaviral mRNA's have eliminated the need for the 5′ cap by replacing this modification with an internal ribosome entry site (IRES) that enables ribosomes to bind downstream of the 5′ end (see, for example, Pelletier, J. et al. (1988) “Internal initiation of translation of eukaryotic mRNA directed by a sequence derived from poliovirus RNA.” Nature 334:320-325.).

As with many viral pathogens, there are several steps in the viral replication cycle of picomavirus that could serve as potential targets for antiviral therapy including, but not limited to i) attachment, ii) endocytosis, iii) uncoating, iv) protein synthesis, v) replication of the viral genome, vi) assembly of viral capsids, vii) maturation of the virion, and viii) lysis of the cell. To that end, several therapeutic compounds and strategies have been developed to combat rhinoviral infection. Interferons have been administered to affect host cell susceptibility to rhinoviral infection (see for example, Hayden, F. et al. (1983) “Intranasal interferon alpha2 for prevention of rhinovirus infection and illness.” Journal of Infectious Disease 148:543-550). Used prophylactically, these compounds act by inducing a variety of proteins that exhibit antiviral activity (e.g. double stranded RNA-dependent protein kinase, 2′,5′adenylate synthetase, and Mx proteins). Prophylactic immunotherapies (e.g. immuno-globulins) designed to offer passive immunity have also been considered as a method to prevent infection by picomaviruses. In a separate category, capsid inhibiting compounds which block viral uncoating and/or viral attachment to host receptors have recently been explored as potential inhibitors of rhinoviral infection. Many of these compounds (e.g. the “WIN” series, Pleconaril) fill the hydrophobic pocket at the base of the viral canyon and increase capsid stability, thus making the virus more resistant to uncoating (see, for example, Rotbart, H. A. (2000) “Antiviral theyrapy for enteroviruses and rhinoviruses.” Antiviral Chemistry and Chemotherapy 11:261-271). Molecules that inhibit RNA replication (i.e. target viral protein 3A, e.g. Enviroxime, Lilly Pharmaceuticals) and/or viral protein processing (e.g. protease inhibitors, see, for example, Wang Q. M. (1999) “Protease inhibitors as potential antiviral agents for the treatment of picomaviral infections.” Prog Drug Res 52:197-219) have also been tested.

While many of the before mentioned compounds have shown promise as antiviral agents in vitro, most have proven limited in in vivo applications. For instance, patients who received interferon one day post infection exhibited no cessation in the development of infection or symptoms (see for instance, Hayden, F. (1983)). Other compounds such as WIN 54954 (a capsid inhibitor) or Enviroxime, have either failed to significantly reduced the number and severity of colds or were discontinued due to poor pharmacokinetics or adverse reactions (see, for example, Turner, R. B. et al. (1993) “Efficacy of oral WIN 54954 for prophylaxis of experimental rhinovirus infection.” Antimicrobial Agents and Chemotherapy 37:297-300; Miller, F. D. et al. (1985) Controlled trial of enviroxime against natural rhinovirus infections in a community.” Antimicrobial Agents and Chemotherapy 27:102-106). Thus, despite the need for new rhinoviral therapeutics and for a greater understanding of the rhinoviral-host interaction, the art to date has not provided an efficient method of exploring the details of the RV infection cycle. The present invention meets this need and provides a methodology for identifying inhibitors of rhinoviral pathogens.

### BRIEF SUMMARY OF THE INVENTION

The present invention relates to methods of assessing viral activity, and from such methods, obtaining perturbagens with viral-related activity. Such perturbagens then are used to obtain viral-related targets, which in turn can be used to identify potential therapeutics. The invention also provides genetic material for the development of gene therapy agents, vectors and host cells.

The present invention provides perturbagen cW985, biologically active fragments, analogs and modifications thereof, and polypeptides consisting essentially of such perturbagen sequences. In other aspects, the invention provides polypeptides having at least 99%, at least 95%, at least 90%, at least 85% or at least 80% sequence identitity or homology with such perturbagens, and in other aspects provides N- and C-terminal fragments of such perturbagens. The invention further provides a composition of such polypeptides in a pharmaceutically acceptable carrier, and for treating a viral-related condition with a therapeutically effective amount of a polypeptide of the invention.

The present invention also provides polypeptides having viral activity that are fused to heterologous sequences, in some aspects a scaffold or more particularly, a fluorescent protein scaffold, and provides polypeptides having viral activity that are chemically modified, or more particularly, radiolabelled, acetylated, glycosylated, or fluorescently tagged. Antibodies to the polypeptides of the invention also are provided.

The present invention further provides polynucleotides encoding perturbagen cW985, biologically active fragments, analogs and modifications thereof, and polypeptides consisting essentially of perturbagen cW985. In other aspects, the invention provides polynucleotides encoding polypeptides having at least 99%, at least 95%, at least 90%, at least 85% or at least 80% sequence identitity or homology with such perturbagens, and in other aspects provides polynucleotides encoding N- and C-terminal fragments of such perturbagens. In some aspects, the polynucleotides are chemically synthesized.

The present invention further provides host cells, vectors, and gene therapy vectors comprising the polynucleotides of the invention. The host cells of the invention further provide for methods for producing polypeptides having viral activity by culturing such host cells and recoving such polypeptides.

The present invention also provides methods for identifying a cellular target that interacts with the polypeptides of the invention. In some aspects, the method is performed in vitro and comprises detecting reporter expression, and in particular aspects, utilizes a yeast two-hybrid assay format. The present invention further provides for the use of such target in screening for putative viral therapeutics, and in some aspects screens for disruption of polypeptide-target pairs. In particular aspects, a combinatorial chemical library is so screened.

### BRIEF DESCRIPTION OF THE DRAWINGS

Figure Legends

FIG. 1. Diagram of the picornavirus genome and translation products.

FIG. 2. Diagram of the prominent steps in the picornaviral lifecycle FIG. 3. Perturbagen screen for isolation of antiviral sequences. HeLa cells containing members of a cDNA expression library are seeded in T175 flasks and infected with RV-14 at a multiplicity of infection (MOI) of 10. Four hours later, a neutralizing antibody is added to the media. At 24 hours the flasks are washed, additional antibody is added, and cultures are shifted to 39° C. to prevent super-infection. At 48 hours dead cells are again removed by washing and live cells are collected by trypsinization and centrifugation. The cDNA inserts are recovered by PCR. Fresh sublibraries are then created and used to infect HeLa cells for additional rounds of enrichment.

FIG. 4. A. Mapping the biologically important region of a perturbagen. Four perturbagens are derived from different breakpoints within the same gene. By mapping the smallest sequence that is common to all four perturbagens (dotted line) it is possible to identify biologically critical regions (black box). B. Critical regions of a gene can be determined by deletion analysis. For instance, a series of N-terminal deletions (dotted line) can be tested for biological activity. In this example, full activity requires a molecule that is longer than deletion 2 but smaller than deletion 1.

FIG. 5. Basic two-hybrid methodology. When bait and prey molecules interact, the Gal4-AD and Gal40-BD binding domains of the Gal4 transcriptional activator are reconstituted. As a result, this functional unit can associate with the Gal1 UAS and induce transcription of the reporter gene (Leu2).

FIG. 6. Four-Hybrid System. Host cell RNA targets are identified through a four-hybrid modification of the original two-hybrid scheme. Expanded region (lower left) pictures interaction between “bait” and “target” RNA molecules.

FIG. 7. LANCE™. In the homogeneous assay, a Cy5 labeled perturbagen binds to an Eu-target molecule in solution. A. When the two molecules are in close proximity, the emissions of the lanthanide chelate can excite Cy5 and give rise to a robust signal. B. In the presence of a small molecule inhibitor, the Cy5-perturbagen-Target-Eu interaction is prevented. Subsequent excitation of Eu results in little or no signal.

FIG. 8. DELFIA™. In the heterogeneous assay, the target is immobilized to a solid support using an Eu labeled monoclonal antibody. Following incubation with the Cy5 labeled perturbagen, the well is washed to remove unbound Cy5. Due to the close proximity of the Eu and Cy5 moieties in the bound complex, excitation of the lanthanide chelate leads to excitation (and emission) of Cy5. In the presence of a small molecule inhibitor (black circles), the Eu-target and Cy5-perturbagen moieties never come in close proximity. In subsequent washes, the free, unbound, Cy5-peptide conjugate is removed and the Eu induced Cy5 signal is insignificant.

FIG. 9. Description of the infection, washing, and harvest conditions used at each cycle to isolate RV-14 anti-viral perturbagens.

FIG. 10. Table showing the percentage of cells surviving RV-14 infection over the course of the selection cycles.

FIG. 11. DNA and peptide sequence of W985.

FIG. 12. Results of experiments testing the ability of W985 to induce resistance to RV-14 infection when placed out-of-frame. A. RV-14 resistance assay comparing 1) the control (pVT352), 2) GFP-W985 (in-frame) and 3) GFP-W985 out-of-frame (OF). B. Western Blot comparing the relative levels of GFP scaffolded W985 in 1) untransduced H1-Hela cells, 2) cells transduced with the control vector (pVT352) expressing only the GFP scaffold, 3) cells expressing GFP-W985 in frame, and 4) cells expressing GFP-W985 out-of-frame. Perturbagen levels were detected using an anti-GFP antibody directed against the scaffold. The expected size of the GFP ORF is larger in the pVT352 vector construct than it is in the W985-OF construct.

FIG. 13. Western Blot of hsp70 expression in W985 transduced cells. HeLa cells were grown at 37° C. (lane 1) or 39° C. (lane 2). Three different neomycin selected transductants in the W985 contig: W904, W909 and W927 were grown at 33° C. (lanes 3-5 respectively). Transductants of the control vector pVT352.1 grown at 33° C. were heat shocked at 45° C. for 30′ (lane 6). A) Coomassie Blue stained gel of cellular protein extracts. B) Western blot stained with anti-hsp70 antibody. The MOI of the retroviral transduction for samples 3, 4, and 5 was 0.9, 1.0, and 3.5 respectively.

FIG. 14. Single Step Growth Curve. A single step growth curve was performed to compare viral production in HeLa cells containing 1) pVT352.1, 2) W985, 3) the highly penetrant cell clone W985hp2, and 4) the cell clone W985hp3.

FIG. 15. Northern Blot Analysis. The time course of viral RNA production was examined in H1-HeLa cells containing either pVT352.1 (control plasmid) or the perturbagen containing cellular subclone (W985hp3).

FIG. 16. Plaque assay results comparing ability of RV-14 to form plaques on the cell lines described in FIG. 15.

FIG. 17. Sequence of oligo primers used to amplify RV-14 cDNA fragments.

FIGS. 18-21. Vector diagrams.

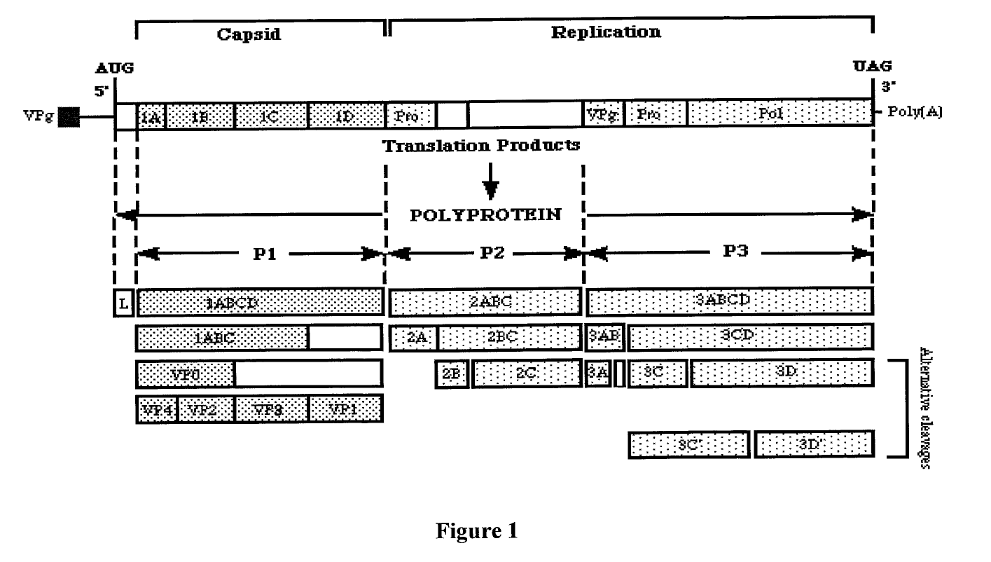

### DEFINITIONS

The terms “perturbagen” or “phenotypic probe” refers to an agent that is proteinaceous or ribonucleic in nature and acts in a transdominant mode to interfere with specific biochemical processes in cells, i.e., through its interaction with specific cellular target(s) or other such component(s), capable of disrupting or activating a particular signaling pathway and/or cellular event. Perturbagens may be encoded by a naturally derived library of compounds such as a cDNA or genomic DNA (gDNA) expression library, or an artificial library comprising synthetic oligonucleotide sequences of a desired length or range of lengths, e.g. a random peptide library. Alternatively, the perturbagen itself can be synthesized using chemical methods. The term “proteinaceous perturbagen” encompasses peptides, oligo- or polypeptides, proteins, protein fragments, or protein variants. Some proteinaceous perturbagens can be as short as three amino acids in length. Alternatively, these agents can be greater than 3 amino acids but less than ten amino acids. Other agents can be greater than ten amino acids but shorter than 30 amino acids in length. Still other agents can be greater than 30 amino acids but less than 100 amino acids in length. Still other agents can be greater than 100 amino acids in length. Naturally occurring proteinaceous perturbagens (i.e. those derived from cDNA or genomic DNA) exhibit a range in size from as little as three to several hundred amino acids. In contrast, synthetic perturbagens (such as those present in a synthetic peptide library) may range in size from three amino acids to fifty amino acids in length and more preferably, from three to 20 amino acids in length, and yet more preferably, about 15 amino acids in length. Similarly, the length of RNA perturbagens can vary. Some RNA perturbagens are as short as 6-10 nucleotides in length. Other RNA perturbagens are between 10 and 50 nucleotides in length. Still other RNA perturbagens are between 50 and 200 nucleotides in length. Other RNA perturbagens are greater than 200 nucleotides in length.

The term “mimetic” refers to a small molecule that (i) exerts the same or similar physiological or phenotypic effect in a bioassay system or in an animal model as does a given perturbagen, or (ii) is capable of displacing a perturbagen from a target in a displacement assay.

The term “small molecule” refers to a chemical compound, for instance a peptide or oligonucleotide that may optionally be derivatized, natural product or any other low molecular weight (less than about 1 kdalton) organic, bioinorganic or inorganic compound, of either natural or synthetic origin. Such small molecules may be a therapeutically deliverable substance or may be further derivatized to facilitate delivery.

The term “target” refers to any cellular component that is directly acted upon by the perturbagen that leads to and/or induces the phenotypic change, detectible for example in a bioassay system.

The terms “library” or “genetic library” refer to a collection of nucleic acid fragments are expressed in a cell and may individually range in size from about nine base pairs to about a ten thousand base pairs. These fragments are generated using a variety of techniques familiar to the art.

The term “sublibrary” refers to a portion of a genetic library that has been isolated by application of a specific screening or selection procedure.

The term “insert” in the context of a library refers to an individual DNA fragment that constitutes a single member of the library.

The terms “reporter gene” and “reporter” refer to nucleic acid sequences (or encoded polypeptides) for which screens or selections can be devised. Reporters may be proteins capable of emitting light, or genes that encode intracellular or cell surface proteins detectible by antibodies. Preferably, the reporter activity may be evaluated in a quantitative manner. Alternatively, reporter genes can confer antibiotic resistance or selectable growth advantages.

The term “gene” refers to a DNA substantially encoding an endogenous cellular component, and includes both the coding and antisense strands, the 5′ and 3′ regions that are not transcribed but serve as transcriptional control domains, and transcribed but untranslated domains such as introns (including splice junctions), polyadenylation signals, ribosomal recognition domains, and the like.

The terms “polynucleotide” or “nucleic acid molecule” are used interchangeably to refer to polymeric forms of nucleotides of any length. The polynucleotides may contain deoxyribonucleotides, ribonucleotides and/or their analogs. Nucleotides may have any three-dimensional structure, and may perform any function, known or unknown. The term “polynucleotide” includes single-, double-stranded and triple helical molecules.

“Oligonucleotide” refers to polynucleotides of between 5 and about 100 nucleotides of single- or double-stranded DNA. Oligonucleotides are also known as oligomers or oligos and may be isolated from genes, or chemically synthesized by methods known in the art. The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A nucleic acid molecule may also comprise modified nucleic acid molecules, such as methylated nucleic acid molecules and nucleic acid molecule analogs. Analogs of purines and pyrimidines are known in the art, and include, but are not limited to, aziridinycytosine, 4-acetylcytosine, 5-fluorouracil, 5-bromouracil, 5-carboxymethylaminomethyl-2-thiouracil, 5-carboxymethyl-arninomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, pseudouracil, 5-pentylnyluracil and 2,6-diaminopurine. The use of uracil as a substitute for thymine in a deoxyribonucleic acid is also considered an analogous form of pyrmidine.

The term “fragment” refers to any portion of a proteinaceous perturbagen that is at least 3 amino acids in length, or any RNA molecule that is at least 5 nucleotides in length. The descriptors “biologically relevant” or “biologically active” refer to that portion of a protein or protein fragment, RNA or RNA fragment, or DNA fragment that encodes either of the two previous entities, that is responsible for an observable phenotype. some portion of an observable phenotype, or for activation of a correlative reporter construct.

The term “variant” refers to biologically active forms of the perturbagen sequence (or the polynucleotide sequence that encodes the perturbagen) that differ from the sequence of the initial perturbagen.

The terms “homology” or “homologous” refers to the percentage of residues in a candidate sequence that are identical with the residues in the reference sequence after aligning the two sequences and introducing gaps, if necessary, to achieve the maximum percent of overlap (see, for example, Altschul, S. F. et al. (1990) “Basic local alignment search tool.” J Mol Biol 215(3):403-10; Altschul, S. F. et al. (1997) “Gapped BLAST and PSI-BLAST: a new generation of protein database search programs.” Nucleic Acids Res 25(17):3389-402). It is understood that homologous sequences can accommodate insertions, deletions and substitutions in the nucleotide sequence. Thus, linear sequences of nucleotides can be essentially identical even if some of the nucleotide residues do not precisely correspond or align. The reference sequence may be a subset of a larger sequence, such as a portion of a gene or flanking sequence, or a repetitive portion of a chromosome.

The term “scaffold” refers to a proteinaceous or RNA sequence to which the perturbagen is covalently linked to provide e.g., conformational stability and/or protection from degradation.

### DETAILED DESCRIPTION OF THE PREFERRED EMBODIMENTS

Agents isolated from the methods described herein have broad potential and application. For example, each RNA or proteinaceous agent (or a mimetic thereof identified through, e.g., routine small molecule screens) may be useful as a direct therapeutic agent in the treatment of picornavirus-induced diseases. With each agent, a corresponding target molecule can be readily identified using standard interaction methodologies such as the two-hybrid technique and/or immunoprecipitation. Such targets are useful in the development of novel drugs for new chemotherapeutic strategies and may provide useful diagnostic tools for profiling the genetic background (genotype) of the particular disease under study.

A. Overview of the Invention

The invention describes the isolation of new and previously unidentified agents that alter a cell's sensitivity to infection by to rhinovirus (RV or RV-14).

The perturbagens described herein were isolated using a phenotypic assay. (see priority document U.S. Pat. No. 5,955,275, “Methods for identifying nucleic acid sequences encoding agents that affect cellular phenotypes,” the disclosure of which is incorporated by reference herein in its entirety. Briefly, the assay identifies agents that alter a cell's susceptibility to killing by RV-14. To accomplish this, a library of polynucleotide sequences is ligated into a standard retroviral expression vector and transferred into a population of cells that are susceptible to rhinoviral infection (e.g. HeLa cells). Subsequently, the library containing cells are challenged with the virus at a multiplicity of infection (MOI) of 10 and screened for sequences that protect the cell from viral-induced cell death (FIG. 3). The assay advantageously identifies one or more relevant sequences from the library in a single experimental procedure. Cells expressing a biologically relevant perturbagen induce a particular phenotype (or correlative activation of a reporter gene), and are then separated from the rest of the population by either centrifugation or high-throughput screening procedures such as Fluorescent Activated Cell Sorting (FACS). FACS machines are particularly attractive in these procedures because they are both highly sensitive and efficient (obtaining screening speeds of approximately 10,000 to up to approximately 65,000 cells or more per minute), thus facilitating identification of biologically relevant sequences that exist at low frequencies within a cell population.

Though there are several conceptual similarities between viral perturbagen screens and previous screens described in, for instance, U.S. Pat. No. 5,955,275, a unique set of practical and theoretical and problems present themselves in developing screens for anti-viral agents. In perturbagen assays based on standard positive selections or Trans-FACS principles (see U.S. Pat. No. 5,955,275 or 5,998,136), both the amount of the agent added to each culture and the length time cells are exposed to said agent, can be tightly regulated. As such, the experimenter can control the fraction of the population that exhibits the phenotype of choice (e.g. cell death, activation of a transcriptionally regulated reporter). For instance, in positive selections designed to identify perturbagens that protect cells from the cytotoxic effects of agents such as cisplatin, the level or length of time to which cells are exposed to said agent can be controlled to improve the chances of identifying perturbagens that block the action of the chemical. In contrast, biological assays designed to identify perturbagens that block the viral life cycle are complicated by the fact that the agent designed to induce the phenotype of choice (e.g. cell death) is capable of self-replication. Thus, though a researcher may add a quantity of virus that is sufficient to infect each cell with only a single copy of the viral genome, subsequent rounds of viral replication and release effectively increase the “concentration” of the viral titer, and thus alter the experimental conditions. For this reason, assays designed to identify viral perturbagens must contain modifications that can control such experimental fluctuations. In some cases, varying the levels of viral agents can be controlled by introducing additional washing steps. In other instances, chemical or biological agents (e.g. antibodies) that neutralize newly released viral particles may be added to the culture to limit further infection of the cells. For instance, specific drugs that intercalate into the viral capsid and neutralize the virus ability to bind to the host receptor or docking molecule can be utilized to prevent supra-infection. In yet another approach, culture conditions such as pH or temperature may be shifted to prevent supra-infection of the cells. It should be noted that due to the large numbers of viral particles being utilized in these experiments and the high rate at which virus spontaneously mutate, it may, in some instances, be necessary to apply two or more of the above mentioned modifications in order to aptly control the viral titer. Thus in some experiments, both additional washing steps, a neutralizing antibody, and temperature shifts, will be incorporated into the screening procedures to limit viral infection.

To identify molecules that alter a cell's ability to resist or deter RV infection, a random primed library of 12×10⁶ clones was constructed from cDNA isolated from placental tissue. This genetic library was transfected into twenty million cervical adenocarcinoma cells (HeLa) that were previously shown to be susceptible to RV infection. Subsequently, the library containing population was expanded nine-fold and then exposed to rhinovirus to identify perturbagens that inhibit the viral lifecycle.

Perturbagen identification may elucidate the function of known host genes, or alternatively may work in a black-box approach to identify new genes, gene products, or cellular targets. Thus in some instances, perturbagens may be encoded by a previously identified gene (or gene fragment thereof). Such a gene may be one whose contribution to the disease pathway has previously been identified (e.g. eIF4G, see, for instance, Haghighat A. et al. (1996) “The eIF4G-eIF4E complex is the target for direct cleavage by the rhinovirus 2A proteinase.” J. Virol 70(12):8444-50). Alternatively, the gene's contribution to the pathway may have been previously unrecognized. In other cases, the perturbagen may be found to have no homology with any previously identified polynucleotide or proteinaceous agent. Such perturbagens may be derived from previously unidentified genes, or alternatively may be random sequences that have the proper conformation and/or chemical characteristics needed to block, alter or modulate one or more components of a pathway(s) that adversely influences the viral lifecycle. In the methodology described herein, no prior knowledge of the perturbagen or of its corresponding gene, gene product or cellular target is necessary. Moreover, because it is possible for multiple perturbagens to assume similar secondary or tertiary structures and/or have shared or related chemistries, two or more variants of the same perturbagen may be identified and isolated from a single library without any additional screening steps. Thus unlike alternative approaches in which a pre-selected candidate molecule is designed, redesigned or manipulated, the methodology described herein has the capacity to evaluate a large number of molecules (e.g., >10⁶) and efficiently identify agents of interest, without preconceived experimental bias.

B. Phenotypic Probes

The invention encompasses both the phenotypic probes (perturbagens) described herewith and the polynucleotide sequences encoding them. As one of ordinary skill appreciates, agents may be described by their amino acid sequence, RNA sequence, or encoding DNA sequence. Alternatively, the agents can be sufficiently described in terms of their identity as isolates of a library that exhibit a particular biological activity.

Perturbagens may be encoded by a variety of genetic libraries, including those developed from cDNA, gDNA, and random, synthetic oligonucleotides synthesized using current available methods in chemistry (see, for example, Caponigro et al. (1998) “Transdominant genetic analysis of a growth control pathway.” PNAS 95:7508-7513; Caruthers, M. H. et al. (1980) Nucleic Acids Symposium, Ser. 7:215-223; Horn, T. et al. (1980) Nucleic Acids Symposium, Ser. 7:225-232; Cwirla, S. E. et al. (1990) “Peptides on phage: a vast library of peptides for identifying ligands.” Proc Natl Acad Sci 87(16):6378-82). Alternatively, the perturbagen itself can be synthesized using chemical methods. For example, peptide and RNA synthesis can be performed using various techniques (Roberge, J. Y. et al. (1995) “A strategy for a convergent synthesis of N-linked glycopeptides on a solid support.” Science 269:202-204; Zhang, X. et al. (1997) “RNA synthesis using a universal base-stable allyl linker.” NAR 25(20): 3980-3983). Automated synthesis may be achieved using commercially available equipment such as the ABI 431A peptide synthesizer (Perkin-Elmer).

In some cases, the polynucleotide sequence encoding a perturbagen represents a fragment of an existing gene. Using currently available software, it is possible to identify the full length cDNA by aligning the perturbagen encoding sequence with pre-existing sequences maintained in, for instance, publicly available genomic and/or EST data bases. In situations where the gene has not been identified, the perturbagen can be readily used to reverse engineer and identify the gene from which the phenotypic probe is derived.

In the case where a perturbagen is encoded by only a portion of a particular gene, the nucleic acid sequence of such a perturbagen may be extended utilizing a partial nucleotide sequence and employing various PCR-based methods known in the art to detect upstream sequences. One such method, restriction site PCR, uses universal and nested primers to amplify unknown sequence from genomic DNA within a cloning vector (Sarkar, G. (1993) “Restriction-site PCR: a direct method of unknown sequence retrieval adjacent to a known locus by using universal primers.” PCR Methods Applic. 2:318-322). Another method, inverse PCR, uses primers that extend in divergent directions to amplify unknown sequence from a circularized template. The template is derived from restriction fragments comprising a known genomic locus and surrounding sequences (see Triglia, T. et al. (1988) “A procedure for in vitro amplification of DNA segments that lie outside the boundaries of known sequences.” NAR. 16:8186). A third method, capture PCR, involves PCR amplification of DNA fragments adjacent to known sequences in human and yeast artificial chromosome DNA (Lagerstrom, M. et al. (1991) “Capture PCR: efficient amplification of DNA fragments adjacent to a known sequence in human and YAC DNA.” PCR Methods Applic. 1:111-119). In this method, multiple restriction enzyme digestions and ligations may be used to insert an engineered double stranded sequence into a region of known sequence before performing PCR. Other methods which may be used to retrieve unknown sequences are known in the art (Parker, J. D. et al (1991) “Targeted gene walking polymerase chain reaction.” NAR. 19:3055-3060). In addition, one may use nested primers and PROMOTERFINDER libraries (Clontech, Palo Alto, Calif.) to walk genomic DNA. This procedure avoids the need to screen libraries and is useful in finding intronlexon junctions. For all PCR based methods, primers may be designed, using commercially available software such as OLIGO 4.06 Primer Analysis software (National Biosciences, Plymouth Minn.) or another appropriate program, to be about 22 to 30 nucleotides in length, to have a GC content of about 50% or more, and to anneal to the template at temperatures of about 68° C. to 72° C.

In one particular embodiment, the invention encompasses proteinaceous perturbagens, biologically active fragments, (N-terminal, C-terminal, or central) or variants thereof. Proteinaceous perturbagens can exert their effects by multiple means and may act on either a host or viral protein or nucleic acid target. For example, a peptide may act by binding and disrupting the interactions between two or more proteinaceous entities within the cell. Alternatively, a peptide perturbagen can, for instance, bind to, and disrupt translation of a viral or host mRNA molecule. As still another alternative, peptide perturbagens may bind to genomic DNA and disrupt gene expression by altering the ability of one or more transcription factor(s) (e.g. activators or repressors) from binding to a critical enhancer/promoter region of the regulatory region of a gene that is necessary for viral replication.

In addition to these intracellular modes of perturbagen action, perturbagens can disrupt the viral lifecycle by acting extracellularly. For example, a particular perturbagen may be designed to be secreted out of the cell where it then exhibits antiviral nature by binding to the rhinoviral capsid “canyon” and thereby disrupts viral-ICAM-1 (receptor) interactions. Alternatively, the perturbagen may, as a result of it's insertion into the cytoplasmic membrane, disrupt the configuration or orientation of the viral receptor and thus prevent either the attachment of the virus to the host cell surface or the injection of the viral genome into the host cytosol. Perturbagens can act by these means or may have other modes of action to disrupt viral replication.

Penetrance is another property of perturbagens. Penetrance is defined as the fraction of cells carrying a perturbagen that exhibit the phenotype in question (i.e. protection against a viral pathogen). Depending upon the background of the assay it may be useful to adjust this number by subtracting out the fraction of cells exhibiting the phenotype when there is no perturbagen present. The penetrance of any given pertubagen can vary depending upon a number of parameters including 1) the cell type it is being expressed in, 2) the vector being used to express the perturbagen, 3) the biological stability (half-life) of the perturbagen or mRNA encoding the perturbagen 4) the concentration of the perturbagen in the cell, as well as other parameters such as the particular strain of the virus that is infecting the cell or, possibly, the extracellular conditions (e.g. pH) in situations where there perturbagen is acting extracellularly. Thus, in some instances a desirable, biologically active perturbagen may present a relatively low rate of penetrance. As one of ordinary skill will appreciate, perturbagens of low penetrance may be obtained and manipulated via standard cycling and/or amplification procedures. Thus, some preferred perturbagens may exhibit as low as 1-2% penetrance. Other preferred perturbagens may exhibit between 2% and 5% penetrance, between 5 and 10% penetrance, 10% and 20% penetrance, between 20% and 50% penetrance, or even in some instances, between 50% and 100% penetrance.

In some instances, the action, penetrance, or biological activity of a perturbagen may be affected in some part by the scaffold to which it is associated. In some cases (for instance, in situations where the agent is shorter than 30 amino acids) the scaffold may drive the perturbagen to adopt a conformation that enhances its biological action. In still other instances, one or more neighboring residues from, e.g., the C-terminus of a scaffold, may act in concert with the perturbagen to enhance the functionality of the molecule. In cases such as these, the complete biologically active sequence may include one or more C-terminal residues derived from the scaffold molecule. Multiple techniques may be used to determine the contribution of the scaffold to the phenotypic effect of any given perturbagen. Initially, perturbagen sequences can be shifted to alternative scaffolds and retested for biological activity. If these procedures result in a significant loss of the perturbagen's activity, a fusion between the perturbagen and, for instance, the 30 most residues from the C-terminus of the scaffold may be linked to a second scaffold molecule and retested for biological activity. Should operations such as these lead to the recovery of lost activity, experiments in which smaller and small portions of the primary scaffold are associated with the perturbagen can be tested.

In other embodiments, the phenotypic probe is an RNA molecule which is itself active (i.e. is not acting through the correlative encoded protein or peptide that results from translation of the RNA). There are multiple mechanisms by which RNA molecules may act to inhibit or activate a biological pathway. In some instances, the RNA perturbagen acts in an antisense mode to disrupt ribonucleic acid transcription or translation of a cellular or viral mRNA target via hybridization to a target ribonucleic acid (Weiss, B. et al.(1999) “Antisense RNA gene therapy for studying and modulating biological processes.” Cell Mol Life Sc.i 55(3):334-58). In this context the term “antisense” refers to any composition containing a nucleic acid sequence which is complementary to the “sense” strand of a particular RNA or DNA target (see, for example, Chadwick, D. R. et al. (2000) “Antisense RNA sequences targeting the 5′ leader packaging signal region of human immunodeficiency virus type-1 inhibits viral replication at post-transcriptional stages of the life cycle.” Gene Therapy 7(16):1362-8.) In other instances, RNA perturbagens may act as a RNA-PRO (RNA-protein) agents, disrupting the viral lifecycle by interacting with proteinaceous components of the virus or cell (see Sengupta, D. J. (1999) “Identification of RNAs that bind to a specific protein using the yeast three-hybrid system.” RNA 5:596-601). In still other instances, RNA agents act as a triplex-forming oligonucleotide (TFO) agents to interact with promoter sequences, exons, introns, or other portions of genomic DNA to (for example) activate transcription of components that interfere with viral replication (see Postel, E. H. et al. (1989) “Evidence that a triplex-forming oligonucleotide binds to the c-myc promoter in HeLa cells, thereby reducing c-myc RNA levels.” PNAS 88: 8227-8231; Svinarchuk, F. et al. (1997) “Recruitment of transcription factors to the target site by triplex-forming oligonucleotides.” NAR 25:3459-3464).

There does not appear to be a necessary correlation between size of a particular RNA (or proteinaceous) perturbagen and penetrance. Instead, the penetrance of perturbagens are dependent upon the perturbagen stability or half-life, the perturbagen's ability to achieve access to the target molecule, and other factors.

Perturbagens may also exhibit cross-reactivity. A variety of host target proteins can contain similarities in both the primary and secondary structure. As a result, one or more of the agents described herein may exhibit affinity for one or more target variants/isoforms present in nature. Similarly, agents identified in the following screens may exhibit affinity for two or more functionally unrelated proteins that contain regions or domains that share homology or related functional groups. Thus, for instance, a perturbagen that recognizes a zinc-binding domain of one protein may also show affinity for the homologous (and functionally equivalent) region of a second protein (see, e.g., Mavromatis K. O. et al. (1997) “The carboxyl-terminal zinc-binding domain of the human papillomavirus E7 protein can be functionally replaced by the homologous sequences of the E6 protein.” Viral Research 52(1):109-18). In cases where such interactions lead to relevant biological phenotypes, the underlying mechanism(s) may differ considerably from those brought about by the original perturbagen-target interactions. Furthermore, in cases where an agent exhibits cross reactivity with secondary targets, said agents may be useful in a broader set of therapeutic and diagnostic applications than originally intended.

Host range is another characteristic of perturbagens. The term “host range” refers to the breadth of potential host cells that exhibit perturbagen-induced phenotypes. In some instances, such as the case where the perturbagen is represented by an apoptosisinducing fragment of BID, the host range is broad, due to the near ubiquitous participation of BID or BID-like agents in the apoptotic pathway of many cells. In contrast, some perturbagens have a very limited host range due to, for instance, the restricted expression of the perturbagen target.

C. Sequence Variants

In another embodiment, the invention includes sequence variants of both the phenotypic probes and the polynucleotide sequences that encode them. Thus, in the case of proteinaceous perturbagens, variants contain at least one amino acid substitution, deletion, or insertion from the original isolated form of the perturbagen that provides biological properties that are substantially similar to those of the initial perturbagen. Similarly, variants of RNA-based phenotypic probes contain at least one nucleotide substitution, deletion, or insertion when compared to the original isolated sequence.

In addition to being described by their respective sequence, variants may also be identified by the relative amounts of homology they have in common with the original perturbagen sequence. Alternatively, a variant of a proteinaceous perturbagen may be described in terms of the nature of an amino acid substitution. “Conservative” substitutions are those in which the substituting residue is structurally or functionally similar to the substituted residue. In non-conservative substitutions, the substituting and substituted residue will be from structurally or functionally different classes. For the purposes herein, these classes are as follows: 1. Electropositive: R, K, H; 2. Electronegative: D, E; 3. Aliphatic: V, L, I, M; 4. Aromatic: F, Y, W; 5. Small: A, S, T, G, P, C; 6. Charged: R, K, D, E, H; 7. Polar: S, T, Q, N, Y, H, W; and Small Hydrophilic: C, S, T. Interclass substitutions generally are characterized as nonconservative, while intraclass substitutions are considered to be conservative. In some instances, variant polypeptides sequences can have 65-75% homology with the original agent. In other embodiments, variants have between 75% and 85% homology with the original agent. In still other embodiments, variants will have between 85% and 95% homology with the original perturbagen agent. In yet other embodiments, variants have between 95% and greater than 99% polypeptide sequence identity with the original perturbagen agent. In some cases, the homology between two perturbagens (variants) is confined to a small region of the molecule (e.g. a motif). Such conserved sequences are often indicative of regions that contain biologically important functions and suggest the perturbagens share a common cellular or viral target. In these situations, while only limited and conservative amino acid changes are desirable within the region of the motif, greater levels of variation can exist in adjacent and more distal portions of the polypeptide.

Like their proteinaceous counterparts, variants of RNA perturbagens may also be described in terms of percent homology. In some instances, the variant ribonucleotide sequences can have 65-75% homology with the original agent. In other embodiments, the variants have between 75% and 85% homology with the original agent or between 85% and 95% homology with the original perturbagen sequence, or even between 95% and greater than 99% sequence identity with the original perturbagen agent. Again, greater variation can, in some embodiments, exist outside an identified region/motif without altering biological activity.

Lastly, in reference to the DNA sequences encoding proteinaceous perturbagens, one who is skilled in the art will appreciate that the degree of variance will depend upon and/or reflect the degeneracy of the genetic code. As one in the art appreciates, a given protein sequence is equivalently encoded by a large number of polynucleotide sequences. Therefore, the invention encompasses each variation of polynucleotide sequence that encodes the given perturbagen, such variations being made in accordance with the standard triplet genetic code as applied to the polynucleotide sequence of each perturbagen. For each proteinaceous perturbagen described by amino acid sequence herein, all such corresponding DNA variations are to be considered as being specifically disclosed.

Variants of phenotypic probes may arise by a variety of means. Some variants may be artifactual and result from, for instance, errors that occur in the process of PCR amplification or cloning of the perturbagen encoding sequence. Alternatively, variants may be constructed intentionally. For instance, it may be advantageous to produce nucleotide sequences encoding perturbagens possessing a substantially different codon usage. Codons may be selected to increase the rate at which expression of the peptide or RNA occurs in a particular prokaryotic or eukaryotic cell in accordance with the frequency with which particular codons are utilized by the host (Berg, O. G. (1997) “Growth rate-optimized tRNA abundance and codon usage.” J Mol Biol 270(4):544-50). Additional reasons for substantially altering the nucleotide sequence encoding proteinaceous perturbagens (without altering the encoded amino acid sequences) include, but are not limited to, producing RNA transcripts that have increased half-life. This may be accomplished by altering a sequence's structural stability (see, for example, Gross, G. et al. (1990) “RNA primary sequence or secondary structure in the translational initiation region controls expression of two variant interferon-beta genes in Escherichia coli.” J Biol Chem. 265(29): 17627-36; Ralston, C. Y. et al. (2000) “Stability and cooperativity of individual tertiary contacts in RNA revealed through chemical denaturation.” Nat Struct Biol. 7(5):371-4), or through addition of untranslated sequences that increase RNA stability/half-life through RNA-protein interactions (see, for example, Wang, W. et al. (2000) “HuR regulates cyclin A and cyclin B1 mRNA stability during cell proliferation.” EMBO J. 19(10):2340-50; Shetty, S. and Idell, S. (2000) “Posttranscriptional regulation of plasminogen activator inhibitor-1 in human lung carcinoma cells in vitro.” Am J Physiol Lung Cell Mol Physiol 278(1):L148-56). Also included the category of intentional variants are those whose sequence has been altered in order to add or deleted sites involved in post-translational modification. Included in this list are variants in which phosphorylation sites, acetylation sites, methylation sites, and/or glycosylation sites have been added or deleted (see, for example, Wicker-Planquart, C. (1999) “Site-directed removal of N-glycosylation sites in human gastric lipase.” Eur J Biochem. 262(3):644-51; Dou, Y. (1999) “Phos-phorylation of linker histone H1 regulates gene expression in vivo by mimicking H1 removal.” Mol Cell. 4(4):641-7).

Variants may also arise as a result of simple and relatively routine techniques involving random mutagenesis or DNA shuffling; procedures that are often used to rapidly evolve perturbagen encoding sequences and allow identification of variants that have increased biological stability or activity (see, for instance, Ner, S. S. et al. (1988) “A simple and efficient procedure for generating random point mutations and for codon replacements using mixed oligonucleotides.” DNA 7:127-134; Stemmer, W. (1994) “Rapid evolution of a protein in vitro by DNA shuffling.” Nature 370:389-391). For instance, in mutagenic PCR, the fragment encoding the perturbagen is PCR amplified under conditions that increase the error rate of Taq polymerase. This is accomplished by i) increasing the MgCl₂ concentrations to stabilize non-complementary pairings, ii) addition of MnCl₂ to diminish template specificity of the polymerase and iii) increasing the concentration of dCTP and dTTP to promote misincorporation of basepairs in the reaction. As a result of this process, the error rate of Taq polymerase may be increased from 1.0×10⁻⁴ errors per nucleotide per pass of the polymerase, to approximately 7×10⁻³ errors per nucleotide per pass. Amplifying a perturbagen-encoding sequence under these conditions allows the development of a library of dissimilar sequences which can subsequently be screened for variants that exhibit improved biological activity.

In addition to variants that are created by artificial or accidental means, natural variants may also exist. For instance, in the course of screening any given genolic or cDNA library, it is possible that a perturbagen, derived from a sequence that exists in multiple copies within the genome (e.g. duplications, repetitive sequences), may be isolated numerous times. Such sequences often contain polymorphisms that result in alterations in the encoded RNA and polypeptide sequence (see, for example, Satoh, H. et al. (1999) “Molecular cloning and characterization of two sets of alpha-theta genes in the rat alpha-like globin gene cluster.” Gene 230(1):91-9) and thus, may represent natural variants of the perturbagen agent. Alternatively, if multiple libraries are utilized to screen for perturbagens and two or more of those libraries are derived from unrelated individuals, it is possible that variants may be isolated as a result of allelic variation (see, for example, Posnett, D. N. (1990) “Allelic variations of human TCR V gene products.” Immunol Today. 11(10):368-73). Variants of phenotypic probes may arise by these and other means.

Variants of any given perturbagen may in some instances exhibit additional biological properties. For instance, perturbagens that previously recognized only a single target may demonstrate broadened specificity, e.g., may bind multiple isoforms or serotypes of a target in response to the alteration of a single amino acid in the perturbagen variant. Similarly, a perturbagen having a specific phenotype in one cell may exhibit additional phenotypes or may exhibit a broader effective host range after making small alterations in perturbagen variant sequence.

D. Biologically Active Fragments

Some embodiments of the invention encompass biologically active fragments of a given proteinaceous or RNA-based perturbagen. Biologically active fragments may be compromised of N-terminal, C-terminal, or internal fragments of peptide perturbagens, or 5′, 3′ or internal fragments of RNA perturbagens. In some instances, the fragment encodes or represents portions of a natural gene. In other instances the fragment is derived from a larger polynucleotide or polypeptide that has no known natural counterpart. In still other instances, biologically active regions of a perturbagen can be artificially synthesized (by chemical or recombinant methods) so that multiple, tandem copies of the phenotypic probe are covalently linked together and expressed. All such biologically active perturbagen fragments are, in turn, encoded by a variety of correlative DNA sequences.

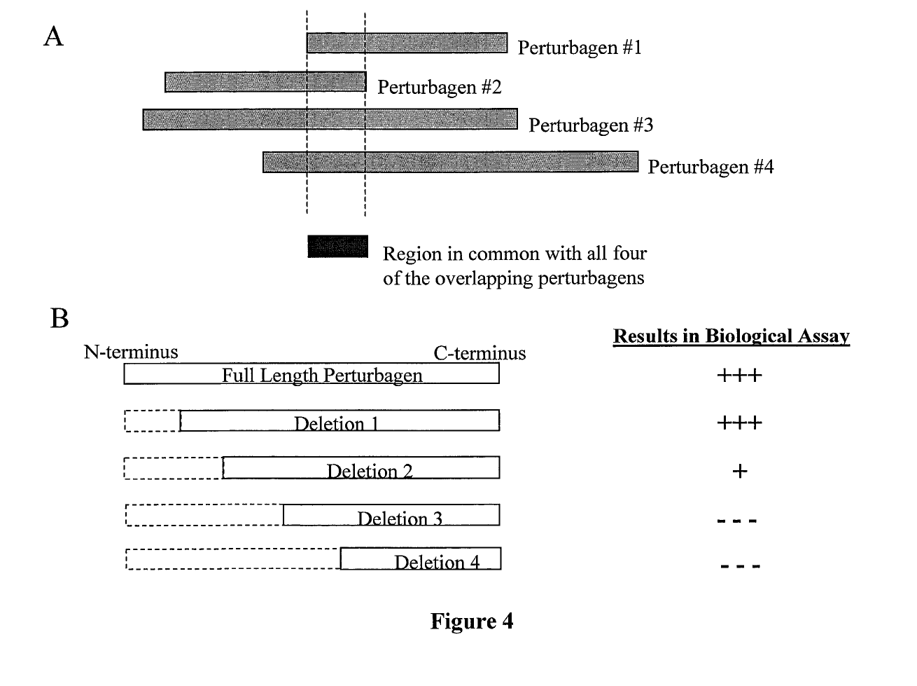

The biologically active portion of a molecule can be identified by several means. In some instances, biological relevant regions can be deduced by simple physical mapping of families of overlapping sequences isolated from a phenotypic assay (Hingorani, K. et al. (2000) “Mapping the functional domains of nucleolar protein B23.” J Biol Chem May 26). For instance, in the course of any given screen, multiple perturbagens, derived from alternative breakpoints of the same gene, may be isolated from one or more genetic libraries. (FIG. 4). The smallest region that is common to all of the perturbagens can demarcate the area of biological importance.

Alternatively, critical regions of a perturbagen can frequently be distinguished by comparing the polynucleotide and/or amino acid sequence of two or more perturbagens that share a common target (see, for example, Grundy, W. N. (1998) “Homology detection via family pair-wise search.” J Comput Biol. 5(3):479-9; Gorodkin, J. et al. (1997) “Finding common sequence and structure motifs in a set of RNA sequences.” Ismb 5:120-3). In this instance, conserved sequences (or motifs) that are identified by this form of analysis often provide important clues necessary to determine biologically important regions of a given molecule. Alternatively, methods that identify biologically relevant regions by altering or deleting regions of the perturbagen molecule can also be used. For instance, the gene encoding a particular perturbagen can be subjected to deletion analysis whereby portions of the gene are removed in a systematic fashion, thus allowing the remaining entity to be retested for its ability to evoke a biological response (see, for example, Huhn, J. et al. (2000) “Molecular analysis of CD26-mediated signal transduction in cells.” Immunol Lett 72(2):127-132; Davezac, N. et al. (2000) “Regulation of CDC25B phosphatases subcellular localization.” Oncogene 19(18):2179-85).

Alternatively, biologically critical regions of a molecule can be identified by inducing mutations in the sequence encoding the polypeptide (see, for example, Ito, Y. et al. (1999) “Analysis of functional regions of YPM, a superantigen derived from gramnegative bacteria.” Eur J Biochem; 263(2):326-37; Kim, S. W. et al. (2000) “Identification of functionally important amino acid residues within the C2-domain of human factor V using alanine-scanning mutagenesis.” Biochemistry 39(8):1951-8.). Subsequent testing of the variants of said molecule for biological activity enables the investigator to identify regions of the perturbagen that are both critical and sensitive to manipulation. Furthermore, molecular probes such as monoclonal antibodies and epitope-specific peptides can be useful in the identification of biologically important regions of a perturbagen (see, for example, Midgley, C. A. et al. (2000) “An N-terminal p14ARF peptide blocks Mdm2-dependent ubiquitination in vitro and can activate p53 in vivo.” Oncogene 19(19):2312-23; Lu, D. et al. (2000) “Identification of the residues in the extracellular region of KDR important for interaction with vascular endothelial growth factor and neutralizing anti-KDR antibodies.” J Biol Chem 275(19):14321-30). In this procedure, probes that bind and thus mask specific regions of a perturbagen can be tested for their ability to block the biological activity of the molecule. These techniques (as well as others) can be used to map the boundaries of any given biologically active residues.

E. Heterologous Sequences

In another embodiment, the invention encompasses all heterologous forms of the phenotypic probes and the polynucleotide sequences encoding them described herewith. In this context, “heterologous sequence(s)” include versions of the perturbagens that are i) scaffolded by other entities, ii) tagged with marker sequences that can be recognized by antibodies or specific peptides, iii) altered to transform post-translational patterns of modification or iv) altered chemically so as to cyclize the molecule for alternative pharmacodynamic/pharmacokinetic properties.

1. Scaffolds

Peptide perturbagens can be fused to protein scaffolds at N-terminal, C-terminal, or internal sites. Similarly, RNA derived perturbagens can be fused to RNA sequences at 5′, 3′ or internal sites. The fusion of a perturbagen to a second entity can increase the relative effectiveness of the perturbagen by increasing the stability of either the messenger RNA (mRNA) or protein of said agent. In some instances, scaffolds may be a relatively inert protein, (i.e. having no enzymatic activity or fluorescent properties) such as hemagglutinin. Such proteins can be stably expressed in a wide variety of cell types without disrupting the normal physiological functions of the cell. In other instances, scaffolds may serve a dual function, e.g., increasing perturbagen stability while at the same time, serving as an indicator or gauge of the level of perturbagen expression. In this case, the scaffold may be an autofluorescent molecule such as a green fluorescent protein (Clontech) or embody an enzymatic activity capable of altering a substrate in such a way that it can be detected by eye or instrumentation (e.g. β galactosidase). For example, in the invention described herein, various molecular techniques that are common to the field are used to link the perturbagen library to, e.g., the C-terminus of a nonfluorescent variant of GFP. “dEGFP” (also referred to as “dead-GFP”) is one such nonfluorescent variant brought about by conversion of Tyr→Phe at codon 66 of EGFP (Clontech). By linking the perturbagen library to this molecule, each library member is fused to a separate dEGFP molecule. Such chimeric fusions can easily be detected by Western Blot analysis using antibodies directed against GFP and are useful in determination of intracellular expression levels of perturbagens. In addition, by modifying the perturbagen sequences or the scaffold to which they are attached with various localization signals, the perturbagen may be directed to a particular compartment within the host cell. For example, proteinaceous perturbagens can be directed to the nucleus of certain cell types by attachment of a nuclear localization sequence (NLS); a heterogeneous sequence made up of short stretches of basic amino acid residues recognized by importins alpha and/or beta.

2. Antibody-Tagged Perturbagens

Perturbagens can be constructed to contain a heterologous moiety (a “tag”) that is recognized by a commercially available antibody. Such heterologous forms may facilitate studies of subjects including, but not limited to, i) perturbagen subcellular localization, ii) intracellular concentration assessment and iii) target binding interactions. In addition, the tagging of a perturbagen may also facilitate purification of fusion proteins using commercially available matrices (see, for example, James, E. A. et al. “Production and characterization of biologically active human GM-CSF secreted by genetically modified plant cells.” Protein Expr Purif. 19(1):131-8; Kilic, F. and Rudnick, G. (2000) “Oligomerization of serotonin transporter and its functional consequences.” Proc Natl Acad Sci U S A. 97(7):3106-11). Such moieties include, but are not limited to glutathione-S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc and HA enable immunoaffinity purification of fusion proteins using commercially available monoclonal and polyclonal antibodies that specifically recognize these epitope tags. Such fusion proteins may also be engineered to contain a proteolytic cleavage site located between the perturbagen sequence and the heterologous protein sequence, so that the perturbagen may be cleaved away from the heterologous moiety following purification. A variety of commercially available kits may be used to facilitate expression and purification of fusion proteins.

3. Chemnically Modified Perturbagens

In addition to the chimeric variants described above, chemical modification encompass a variety of modifications including, but not limited to, perturbagens that have been radiolabeled with ³²P or ³⁵S, acetylated, glycosylated, or labeled with fluorescent molecules such as FITC or rhodamine. These modifications may be directly imposed on the perturbagen itself (see, for example, Shuvaev, V. V. et al. (1999) “Glycation of apolipoprotein E impairs its binding to heparin: identification of the major glycation site.” Biochim Biophys Acta 1454(3):296-308; Dobransky, T. et al. (2000) “Expression, purification and characterization of recombinant human choline acetyltransferase: phosphorylation of the enzyme regulates catalytic activity.” Biochem J. 349(Pt 1): 141-151). Alternatively, changes may be made to the polynucleotide sequence encoding the perturbagen so as to alter the pattern of phosphorylation, acetylation, or glycosylation. In addition, the term “chemical modification” may include methods that lead to cyclization of peptides in order to alter membrane permeability and/or pharmacodynamicpharmacokinetic properties (see, for example, Borchardt, R. T. (1999) “Optimizing oral adsorption of peptides using prodrug strategies.” J Control Release 62(1-2):231-8.).

F. Hybridization

The invention also encompasses polynucleotide sequences that are capable of hybridizing to the claimed polynucleotide sequences encoding phenotypic probes and said variants of such entities described previously, under various conditions of stringency. Such reagents may be useful in i) therapeutics, ii) diagnostic assays, iii) immunocytology, iv) target identification, and v) purification. For example, if the sequence encoding a particular perturbagen is introduced into a subject for gene therapeutic purposes, it may be necessary to monitor the success of integration and the levels of expression of said agent by Southern and Northern Blot analysis respectively (Pu, P. et al. (2000) “Inhibitory effect of antisense epidermal growth factor receptor RNA on the proliferation of rat C6 glioma cells in vitro and in vivo.” J Neurosurg. 92(1):132-9). In other instances, hybridization may be used as a tool to define or describe a perturbagen variant or fragment. Alternatively a hybridizing sequence thus may have direct relevance as an anti-viral mimetic or other such therapeutic agent.

The term “hybridization” refers to any process by which a strand of nucleic acid binds with a complementary or near-complementary strand through base pairing. There are several parameters that play a role in determining whether two polynucleotide molecules will hybridize including salt concentrations, temperature, and the presence or absence of organic solvents. For instance stringent salt concentrations will ordinarily be less than about 750 mM NaCl and 75 mM trisodium citrate, preferably less than about 500 mM NaCl and 50 mM trisodiium citrate, and most preferably less than about 250 mM NaCl and 25 mM trisodium citrate. Low stringency hybridization can be obtained in the absence of organic solvent (e.g. formamide) while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent and the inclusion or exclusion of carrier DNA are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide and 100 ug/ml denatured salmon sperm DNA (ssDNA). In a most preferred embodiment, hybridization will occur at 42° C. in 250 mM NaCl, 25 mM trisodium citrate, 1% SDS, 50% formamide and 200 ug/ml denatured ssDNA. Useful variations on these conditions will be readily apparent to those skilled in the art.

The washing steps that follow hybridization can also vary greatly in stringency. Wash stringency conditions can be defined by salt concentration and by temperature. As above, wash stringency can be increased by decreasing salt concentration or by increasing temperature. For example, stringent salt concentrations for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include temperatures of at least about 25° C., more preferably of at least about 42° C., and most preferably of at least about 68° C. In a preferred embodiment, wash steps will occur at 25° C. in 30 mM NaCl, 3 mM trisodium citrate and 0.1% SDS. In a more preferred embodiment, wash steps will occur at 42° C. in 15 mM NaCl, 1.5 mM trisodium citrate and 0.1% SDS. In a most preferred embodiment, wash steps will occur at 68° C. in 15 mM NaCl, 1.5 mM trisodium citrate and 0.1% SDS. Additional variations on these conditions will be readily apparent to those skilled in the art.

G. Expression Vectors

The DNA sequence encoding each perturbagen or target (or variant or fragment thereof) may be inserted into an expression vector which contains the necessary elements for transcriptional/translational control in a selected host cell. Thus the DNA sequence may be expressed for, e.g., testing in a bioassay such as those described herein, or in a binding assay such as those described herein, or for production and recovery of the proteinaceous agent. Methods which are well known to those skilled in the art are used to construct expression vectors containing sequences encoding the perturbagens and the appropriate transcriptional and translational control elements. These methods include in vitro recombinant DNA techniques, synthetic techniques, and in vivo genetic recombination (see Sambrook, J. et al. (1989) “Molecular Cloning, A Laboratory Manual”, Cold Spring Harbor Press, Plainview N.Y.).

Exemplary expression vectors may include one or more of the following: (i) regulatory sequences, such as enhancers, constitutive and inducible promoters, and/or (ii) 5′ and 3′ untranslated regions, and/or (iii) mRNA stabilizing sequences or scaffolds, for optimal expression of the perturbagen in a given host. For instance, intracellular perturbagen levels can be modulated using alternative promoter sequences such as CMV, RSV, and SV40 promoters, to drive transcription (see, for example, Zarrin, A. A. et al. (1999) “Comparison of CMV, RSV, SV40 viral and Vlambdal cellular promoters in B and T lymphoid and non-lymphoid cell lines.” Biochim Biophys Acta. 1446(1-2):135-9). Alternatively, inducible promoter systems, (e.g. ponesterone-induced promoter (PIND, Invitrogen, see Dunlop, J. et al. (1999) “Steroid hormone-inducible expression of the GLT-1 subtype of high-affinity I-glutamate transporter in human embryonic kidney cells.” Biochem Biophys Res Commun. 265(1):101-5), tissue specific enhancers (see Scharf, D. et al. (1994) Results Probl. Cell Differ. 20:125-162), or scaffolding molecules (see, for example, see Abedi, M. et al. (1998), “Green fluorescent protein as a scaffold for intracellular presentation of peptides.” Nucleic Acid Research 26(2):623-630) can be used to modulate intracellular perturbagen levels.

A variety of paired expression vector/host systems may be utilized to contain and express sequences encoding the perturbagens. As one of ordinary skill will appreciate, the selection of a given system is dictated by the purpose of expression: e.g., bioassay, binding assay, or production of proteinaceous product for subsequent isolation and purification. Such systems include, but are not limited to, microorganisms such as bacteria transformed with recombinant bacteriophage, plasmid or cosmid DNA expression vectors; yeast transformed with yeast expression vectors, insect cell systems infected with viral expression vectors (e.g. baculovirus), plant cell systems transformed with viral expression vectors (e.g. tobacco mosaic virus, TMV) or with bacterial expression vectors (e.g. Ti or pBR322 plasmids; or mammalian cell systems (e.g. COS, CHO, BHK, 293, 3T3) harboring recombinant expression constructs containing promoters derived from the genome of mammalian cells (e.g., metallothionine promoter) or from mammalian viruses (e.g., the adenovirus late promoter; the vaccinia virus 7.5 K promoter). The host cell employed does not limit the invention.

In bacterial systems, a number of cloning and expression vectors may be selected depending upon the use intended for polynucleotide sequences encoding the perturbagens or targets. For example, routine cloning, subcloning, and propagation of polynucleotide sequences encoding perturbagens can be achieved using a multifunctional E. coli vector such as PBLUESCRIPT (Stratagene, La Jolla Calif.). Ligation of sequences encoding perturbagens into the vector's cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. (see e.g., Van Heeke, G. and Schuster, S. M. (1989) “Expression of human asparagine synthetase in Escherichia coli.” J. Biol. Chem. 264:5503-5509). When large quantities of perturbagens or targets are needed, e.g. for the production of antibodies, vectors which direct high level expression of perturbagens may be used. Exemplary vectors feature the strong, inducible T5 or T7 bacteriophage promoter; the E. coli expression vector pUR278 (Ruther et al., EMBO J., 2:1791-94 (1983)), in which the gene protein coding sequence may be ligated individually into the vector in frame with the lac Z coding region so that a fusion protein is produced; pIN vectors (Inouye & Inouye, Nucleic Acids Res., 13:3101-09 (1985); Van Heeke et al., J. Biol. Chem., 264:5503-9 (1989)); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned anaphylatoxin C3a receptor gene protein can be released from the GST moiety.

Yeast expression systems may also be used for production of perturbagens and targets. A number of vectors containing constitutive or inducible promoters such as alpha factor, alcohol oxidase, and PGH promoters, may be used in the yeast Saccharomyces cerivisiae or related strains. In addition, such vectors can be designed to direct either the secretion or intracellular retention of expressed proteins and enable integration of foreign sequences in the host genome for stable propagation. (see, e.g. Bitter, G. A. et al. (1987) “Expression and secretion vectors for yeast.” Methods Enzymology. 153:516-544; and Scorer, C. A. et al. (1994) “Rapid selection using G418 of high copy number transformants of Pichia pastoris for high-level foreign gene expression.” Biotechnology 12:181-184).

In mammalian host cells, a number of viral-based expression systems may be utilized. In cases where an adenovirus is used as an expression vector, the gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing gene protein in infected hosts. (e.g., see Logan et al., Proc. Natl. Acad. Sci. USA, 81:3655-59 (1984)). Specific initiation signals may be used to achieve more efficient translation of sequences encoding the perturbagen or target. Such signals include the ATG initiation codon and adjacent sequences, e.g. the Kozak sequence. In cases where sequences encoding the perturbagen or target and its initiation codon and upstream regulatory sequences are inserted into the appropriate expression vector, no additional transcriptional or translational control signals may be needed. However, in cases where only coding sequence is inserted, exogenous translational control signals including an in-frame ATG initiation codon are provided by the vector. Furthermore, the initiation codon must be in phase with the reading frame of the desired coding sequence to ensure translation of the entire insert. Such exogenous translational elements and initiation codons may be of various origins, both natural and synthetic. The efficiency of expression may be enhanced by the inclusion of appropriate transcription enhancer elements, transcription terminators, etc. (see Bitter, et al., Methods in Enzymol., 153:516-44 (1987)). Alternatively, many of these elements are not required in vectors that are specific for RNA-based perturbagens. Instead, sequences that stabilize the RNA transcript or direct the RNA sequence to a particular compartment will be included (see, for instance, Wood Chuck post transcriptional regulatory element, WPRE, Zufferey, R. et al. (1999) “Woodchuck hepatitis virus posttranscriptional regulatory element enhances expression of transgenes delivered by retroviral vectors.” J Virol 73(4):2886-92).

Plant systems may also be used for expression of perturbagens and targets. Transcription of sequences encoding perturbagen or target sequences may be driven by viral promoters, e.g. the 35S and 19S promoters of CaMV used alone or in combination with the omega leader sequence from TMV (Takamatsu, N. (1991) “Deletion analysis of the 5′ untranslated leader sequence of tobacco mosaic virus RNA.” J Virology 65:1619-22). Alternatively, plant promoters such as the small subunit of RUBISCO or heat shock promoters may be used. (see, for example, Coruzzi, G. et al. (1984) “Tissue-specific and light-regulated expression of a pea nuclear gene encoding the small subunit of ribulose-1,5-bisphosphate.” EMBO J. 3:1671-80; Broglie, R. et al. (1984) “Light-regulated expression of a pea ribulose-1,5-bisphosphate carboxylase small subunit gene in transformed plant cells.” Science 24:838-843).

In an insect system, Autographa californica nuclear polyhedrosis virus (AcNPV) is used as a vector to express foreign genes. The virus grows in Spodoptera frugiperda cells. The gene coding sequence may be cloned individually into non-essential regions (for example the polyhedrin gene) of the virus and placed under control of an AcNPV promoter (for example the polyhedrin promoter). Successful insertion of gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (see, e.g., Smith, et al., J. Virol. 46: 584-93 (1983); U.S. Pat. No. 4,745,051).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138, etc.

The selected construct can be introduced into the selected host cell by direct DNA transformation or pathogen-mediated transfection. The terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Preferred technologies for introducing perturbagens into mammalian cells include, but are not limited to, retroviral infection as well as transformation by EBV or similar episomally-maintained viral vectors (Makrides, S. C. (1999) “Components of vectors for gene transfer and expression in mammalian cells.” Protein Expr Purif 17(2):183-202). Other suitable methods for transforming or transfecting host cells can be found in Maniatis, T. et al (“Molecular Cloning: A Laboratory Manual.” Cold Spring Harbor Laboratory Press) and other standard laboratory manuals.

For long term production of recombinant proteins in mammalian systems, stable expression of proteinaceous sequences in cell lines is preferred. For example, sequences encoding targets can be transformed or introduced into cell lines using expression vectors which may contain viral origins of replication and/or endogenous expression elements and a selectable marker gene on the same or on a separate vector. Alternatively, cells can be transfected using, for instance, retroviral, adenoviral, or adeno-associated viral agents as delivery systems for the perturbagen. For example, retroviral vectors (e.g. LRCX, Clontech) may be used to introduce and express perturbagens in a variety of mammalian cell cultures. Such vectors may rely on the virus' own 5′ LTR as a means of driving perturbagen expression or may utilize alternative promoters/enhancers (e.g. those of CMV, RSV and SV40, PIND) to regulate perturbagen or target expression levels.

In a preferred embodiment, timing and/or quantity of expression of the recombinant protein can be controlled using an inducible expression construct. Inducible constructs and systems for inducible expression of recombinant proteins will be well known to those skilled in the art. Examples of such inducible promoters or other gene regulatory elements include, but are not limited to, tetracycline, metallothionine, ecdysone, and other steroid-responsive promoters, rapamycin responsive promoters, and the like (No, et al., Proc. Natl. Acad. Sci. USA, 93:3346-51 (1996); Furth, et al., Proc. Natl. Acad. Sci. USA, 91:9302-6 (1994)). Additional control elements that can be used include promoters requiring specific transcription factors such as viral, particularly HIV, promoters. In one in embodiment, a Tet inducible gene expression system is utilized. (Gossen et al., Proc. Natl. Acad. Sci. USA, 89:5547-51 (1992); Gossen, et al., Science, 268:1766-69 (1995)). Tet Expression Systems are based on two regulatory elements derived from the tetracycline-resistance operon of the E. coli Tn10 transposon the tetracycline repressor protein (TetR) and the tetracycline operator sequence (tetO) to which TetR binds. Using such a system, expression of the recombinant protein is placed under the control of the tetO operator sequence and transfected or transformed into a host cell. In the presence of TetR, which is co-transfected into the host cell, expression of the recombinant protein is repressed due to binding of the TetR protein to the tetO regulatory element. High-level, regulated gene expression can then be induced in response to varying concentrations of tetracycline (Tc) or Tc derivatives such as doxycycline (Dox), which compete with tetO elements for binding to TetR. Constructs and materials for tet inducible gene expression are available commercially from CLONTECH Laboratories, Inc., Palo Alto, Calif.

When used as a component in an assay system, the gene protein may be labeled, either directly or indirectly, to facilitate detection of a complex formed between the gene protein and a test substance. Any of a variety of suitable labeling systems may be used including but not limited to radioisotopes such as ¹²⁵I; enzyme labeling systems that generate a detectable calorimetric signal or light when exposed to substrate; and fluorescent labels. Where recombinant DNA technology is used to produce the gene protein for such assay systems, it may be advantageous to engineer fusion proteins that can facilitate labeling, immobilization and/or detection.

Indirect labeling involves the use of a protein, such as a labeled antibody, which specifically binds to the gene product. Such antibodies include but are not limited to polyclonal, monoclonal, chimeric, single chain, Fab fragments and fragments produced by a Fab expression library.

In some instances, a preliminary selection is performed to verify that the host cells have been successfully transformed/transfected. Following the introduction of the vector, cells are allowed to grow in enriched media, and are then switched to selective media. The selectable marker confers resistance to the selective agent, and thus, only those cells that successfully express the introduced sequences survive in the selective media. Any number of selection systems may be used to recover transformed cell lines. These include, but are not limited to, the herpes simplex virus thymidine kinase and adenine phosphoribosyltransferase genes, for use in tk- or apr-cells, respectively (see e.g. Wigler, M. et al. (1977) “Transfer of purified herpes virus thymidine kinase gene to cultured mouse cells.” Cell 11:223-32; Lowy, I. et al. (1980) “Isolation of transforming DNA: cloning the hamster aprt gene.” Cell 22:817-23). Also antimetabolite, antibiotic, or herbicide resistance can be used as the basis for selection. For example, dhfr confers resistance to methotrexate,; neo confers resistance to the aminoglycosides, neomycin and G-418, and als and pat confer resistance to chlorsulfuron and phosphinotricin acetyltransferase, respectively. (see Wigler, M. et al. (1980) “Transformation of mammalian cells with an amplifiable dominant-acting gene.” PNAS 77:3567-70; Colbere-Garapin, F. et al (1981) “A new dominant hybrid selective marker for higher eukaryotic cells.” J. Mol. Biol. 150:1-14). Additional selectable genes have been described, e.g. trpB and hisD, which alter cellular requirements for metabolites. Visible markers, e.g. anthocyanins, green, red or blue fluorescent proteins (Clontech), B glucuronidase and its substrate B glucuronide, or luciferase and its substrate luciferin, may also be used. Resistant clones containing stably transformed cells may be propagated using tissue culture techniques appropriate to the cell type.

Host cells transformed/transfected with nucleotide sequences encoding for the perturbagen of interest may be cultured under conditions suitable for the expression and recovery of the protein from cell culture. For example, the protein produced by a transformed transfected cell may be secreted when the selected expression vector incorporates signal sequences that direct secretion of the perturbagen through a prokaryotic or eukaryotic cell membrane.

Signal sequences also may be selected so as to direct the perturbagen to a particular intra-cellular compartment (Bradshaw, R. A. (1989) “Protein translocation and turnover in eukaryotic cells.” Trends Biochem Sci 14(7):276-9). Perturbagen sequences may be isolated or purified from recombinant cell culture by methods heretofore employed for other proteins, e.g. native or reducing SDS gel electrophoresis, salt precipitation, isoelectric focusing, immobilized pH gradient electrophoresis, solvent fractionation, and chromatography such as ion exchange, gel filtration, immunoaffinity, and ligand affinity.

H. Host Cells

Host cell lines for use in the methodology described herein typically embody a number of desirable traits such as 1) short cell cycle (i.e. 20-36 hr. doubling time), 2) amenability to high throughput procedures (e.g. FACS) without undue loss of membrane integrity or viability, 3) susceptibility to standard techniques designed to introduce foreign constructs (DNA) into the cell, 4) high susceptibility to viral infection and 5) exhibition of a readily selected phenotype (or its correlative marker gene expression). As one non-limiting example, the cell line is a particular subline of the cervical adenocarcinoma cell line, HeLa. The H1-HeLa subline is highly susceptible to rhinoviral infection. In addition, the cells are amenable to retroviral infection and other methods of introducing foreign genetic materials and can express/maintain said materials for long periods of time using a variety of selectable markers common to the field (e.g. neomycin, puromycin). HeLa cells have two other properties that are useful in this selection. First they can be grown as adherent cells (i.e. cells that replicate and spread across the surface of a suitable tissue culture flask). Second they are capable of long term growth at temperatures that are both permissive and non-permissive for viral reproduction. Though this trait is not essential for the identification of perturbagens that inhibit viral induced cell death, temperature shifting can be useful in restricting secondary infection of cells by virus that have been shed/released by neighboring cells. In addition, it should be noted that in some cases, host cell lines that have been artificially designed to be receptive to viral infection, may be used. In these cases, the viral receptor (e.g. ICAM-1 or LDLR) can be transformed into the cell line of choice.

In addition to cell lines that are receptive to viral infection, sublines that are resistant to viral infection are also useful in testing and optimizing enrichment procedures.

I. Viral Strains

Viral strains for use in the methodology described herein should also embody a range of desirable traits. To begin with, it is advantageous if the viral strains are easily maintained, stored, and grown in the laboratory and have burst sizes (the number of infective virus particles released from an infected cell) that are sufficiently large so as to create viral supernatants that have high titers. If available, attenuated lines which readily infect host cells under artificial conditions (e.g. tissue culture) but place laboratory workers under a minimal level of risk, are also preferred. Alternatively, in cases where attenuated lines are unavailable, genes encoding critical viral functions (e.g. one or more genes involved in viral packaging) can be deleted from the viral genome and provided separately. In this way, the viral particles shed from the infected host cell are crippled and unable to replicate. If possible, the virus should be capable of infecting a broad range of host cells and infection should result in either the death of the host or expression of some other readily identifiable phenotypic change such as expression of a cell surface marker that is recognizable by an antibody. In addition, viral strains that are temperature sensitive, or to which neutralizing antibodies have been developed, are highly desirable. As mentioned previously, restrictive temperatures and neutralizing antibodies are useful in limiting secondary infections that result from viruses released from infected neighboring cells.

As one non-limiting example, the viral strain used in these experiments is the rhinovirus-14 serotype (RV-14, ATCC VR-284). The RV-14 strain can be easily propagated in several human diploid cells including HeLa cells (ATCC CCL-2) and WI-38 cells (ATCC CCL-75) and grows readily (˜8 hour life cycle) at 33° C. Both higher temperatures (e.g.39° C.) and neutralizing antibodies have been shown to limit viral replication (see, for example, Conti, C. et al. (1999) “Antiviral Effect of Hyperthermic Treatment in Rhinovirus Infection.” Antimicrobial Agents and Chemotherapy 43:822; Sherry, B. et al (1986)“Use of monoclonal antibodies to identify four neutralization immunogens on a common cold picornavirus, human rhinovirus 14.” J Virol. 57(1):246-57). Furthermore RV-14 is cytotoxic, thus providing a readily recognizable phenotype for positive screening.

J. Screening for Biological Activity

The phenotypic assay described herein selects for perturbagens that inhibit virusinduced cell death. The procedures used to screen libraries for perturbagens include: i) introducing perturbagen encoding sequences (expression libraries) into the host cell line, ii) infecting said cells with the virus of interest and growing said cells under the appropriate conditions necessary to identify perturbagens that inhibit the viral lifecycle; iii) separating live cells (containing potential anti-viral perturbagens) from dead and/or dying cells; iv) re-isolating perturbagen encoding sequences from live cell populations by various techniques (e.g. PCR); v) enriching for perturbagens by recycling said sequences through the screen; and optionally vi) performing secondary assays to test specificity and scope of the anti-viral agent(s). By performing these steps, the methodology can identify perturbagens that inhibit a number of steps in the viral lifecycle. Depending up on the particular assay used, these steps include, but are not limited to i) release of the viral RNA into the cytosol; ii) translation of the viral genome; iii) cleavage of the viral polyprotein by viral-encoded proteases; iv) replication of the viral genome; v) capsid and virion assembly; vi) maturation of the virion; and vii) exit of the virion from the host cell.

Various methods and instrumentation familiar to those who are skilled in the art are used to screen and test perturbagens. The media, supplements, and reagents used in culturing, packaging, and maintenance of (for instance) HeLa cells, HS293gp packaging cell lines, and additional lines (e.g. WI-38) can be purchased from a variety of commercial and noncommercial sources (Life Technologies, Clonetics, Cocalico Biologicals Inc., ATCC.). It should be noted that although a particular set of procedures and media formulations are used in the work described herein, alternatives may be substituted with little or no effect. For instance, in most cases, retroviral packaging was accomplished using Lipofectamine. Though this is the preferred method of introducing retroviral vectors into 293gp packaging cells, alternative procedures such as the CaCl₂ method of packaging may be used. In addition, molecular techniques used in these procedures such as genomic DNA isolation, PCR amplification, DNA endonculease digestion, ligation, cloning, and sequencing utilize common reagents that are supplied commercially (see, for example, Qiagen, New England BioLabs, Stratagene).

Cell sorting and analysis is performed on a Coulter EPICS Elite Cell Sorter using EXPO software. Again, alternative reagents and equipment, such as the MoFlo^{R} High-Speed Cell Sorter (Cytomation), are compatible with these procedures and may be substituted with little or no effect.

To identify agents that inhibit viral-induced cell death, a retroviral library is first introduced into, e.g. HeLa cells. In some cases the cells are then grown under selective conditions which eliminate cells that do not contain a retroviral (perturbagen) insert. This is achieved by growing the cells in the presence of e.g. neomycin or puromycin which selects for a resistance gene carried by the retroviral vector. If sufficiently high titer retroviral stocks are available so that greater than, for example, 60% of the cells carry at least one retroviral construct, then this step is not necessary. Subsequently the HeLa cells are infected with sufficient quantities of virus e.g. RV-14 to ensure that the majority of the cells are challenged with the pathogen. The nominal enrichment for a perturbagen which blocks viral cytotoxicity is the ratio of the fraction of cells carrying a perturbagen that survive virus challenge to the fraction of control cells that survive virus challenge. Given that the penetrance (percent survivors) of a perturbagen may be only 5-10%, the denominator of the enrichment ratio might be as high as ten or twenty. In this case the numerator of the enrichment factor should be at least 100 to give reasonable enrichment per cycle. Thus the goal of the infection step should be to kill greater than 99% of the cells. For example, if an interesting perturbagen has a survivor rate of roughly 30% under the same conditions in which cells lacking the perturbagen survive at a rate of 0.5%, then in the initial rounds of a selection, when the perturbagen is rare, it's enrichment is 30% divided by 0.5% or 60×.

The distribution of virus among infected cells is governed by the Poisson process and in particular the fraction F of uninfected cells is given by:

F=e^{-MOI}

where MOI is the Multiplicity of Infection. This formula predicts that the smallest possible dose of virus necessary to achieve 99% killing occurs at an MOI of 4.6. However it is necessary to empirically determine the MOI required to achieve any given level of killing. In particular a simple Poisson model may not accurately describe the fraction of survivors at high MOI.

After a proscribed period of incubation that is determined by the time required for infection, the remaining free virus present in the media is eliminated by addition of a neutralizing antibody. Subsequently, prior to the time when the cells would normally lyse and release additional virus into the media, the culture is washed, treated with a second, fresh aliquot of neutralizing antibody, and shifted to a non-permissive temperature that limits the possibility of secondary infections. Adherent cells that are able to resist the cytotoxic effects of RV-14 are then removed from the solid support by trypsinization, and collected by centrifugation. Alternatively, further enrichment of live cells can be obtained by staining cells with any one of a number of vital dyes (e.g. propidium iodide) and then separating viable and non-viable populations by FACS. To complete the cycle, the perturbagen encoding sequences present in the RV-14 resistant cells are then retrieved, repackaged in a retroviral carrier, and recycled through the screen to further enrich for biologically active sequences that protect the cell against RV infection. Again, it should be emphasized that alternative procedures to the ones described above can be practiced. For instance, the timing of application and quantity of the infectious viral agent can vary from experiment to experiment. In some instances, a single infection will be sufficient while in other experiments, double (or even triple) infections may be useful. In other experiments, it may be desirable to identify perturbagens that inhibit a particular step in viral replication. To accomplish this, the methodology may take on additional complexities such as, for instance, transcriptionally-regulated reporter constructs or protease-sensitive reporter molecules to identify perturbagens with unique biological properties.

Several methods may be used to retrieve the perturbagen sequences from cells that have been sorted. For instance, perturbagen-encoding sequences may be recovered by PCR (see, for example, Schott, B. (1997) “Efficient recovery and regeneration of integrated retroviruses.” Nucleic Acids Res. 25(14):2940-2). To accomplish this, genomic DNA (derived from cells taken from the FACS sorting procedures is used as the template for PCR amplification. Using oligonucleotide primers that flank the perturbagen encoding sequence, complex mixtures with diversities of greater than 50,000 can be amplified efficiently. These sequences can subsequently be re-cloned into a retroviral vector, and introduced into a fresh population of, e.g., HeLa cells for additional rounds of screening. Alternatively, retrieval of the perturbagen may be accomplished by reactivating the inserted retroviral vector that contains the perturbagen-encoding sequence. Specifically, host cells containing the perturbagen-encoding (non-infective) retrovirus are transformed with sequences that encode the necessary retroviral gag, pol and envelope proteins. As a result of these procedures, infective retroviral virions that contain the perturbagen-encoding sequences are released and can be isolated in the form of a viral supernatant. These supernatants can then be used to infect fresh populations of, e.g., HeLa cells to recycle the sequences through the screen for additional enrichment.

Secondary viral strains and cell lines may optionally be employed to test individual perturbagens for the ability to protect cells from the cytotoxic effects of viral pathogens. For instance, perturbagens that protect HeLa cells from RV-14 infection can be tested in alternate host backgrounds (e.g. WI-38 cells, ATCC CCL-75) to better understand the host-range and mechanism of the perturbagen. Alternatively, these very same perturbagens can be tested against additional serotypes from both the major and minor classes of rhinovirus to study whether the action of the perturbagen(s) are limited to the RV-14 pathogen. Furthermore, because the rhinoviral structure and lifecycle is closely paralleled by other members of the picornavirus family (e.g. enteroviruses , polio virus) it is reasonable to test the effects of perturbagens on the reproduction of these other viruses. To accomplish this, the perturbagen will be introduced into HeLa cells (or alternative host strains such as Rhesus monkey kidney cells ATCC: LLC-MK2, CCL-7.1) and challenged with other members of the picornavirus family (e.g. Coxsackievirus B2, ATCC#: VR-29; poliovirus, ATCC#: VR193).

K. Cellular Targets

In other embodiments, the invention encompasses the polypeptide, ribonucleotide, or polynucleotide sequence of the target (or fragment of each target) that is identified with each perturbagen agent, as well as the gene encoding each target and relevant fragments of said gene.

Targets of specific perturbagens may be identified by several means. For instance, peptide perturbagens can be modified with homo- or hetero-bifunctional coupling reagents and targets can be identified by chemical cross-linking techniques (see, for example, Tzeng, M. C. et al. (1995) “Binding proteins on synaptic membranes for crotoxin and taipoxin, two phospholipases A2 with neurotoxicity.” Toxicon. 33(4):451-7; Cochet, C. et al. (1988) “Demonstration of epidermal growth factor-induced receptor dimerization in living cells using a chemical covalent cross-linking agent.” J Biol Chem. 263(7):3290-5). Alternatively, one may use various techniques in column affinity chromatography or immunoprecipitation as a method of isolating and identifying target molecules (see, for example, Hentz, N. G. and Daunert, S. (1996) “Bifunctional fusion proteins of calmodulin and protein A as affinity ligands in protein purification and in the study of protein-protein interactions.” Anal Chem. 68(22):3939-44). In yet another example, a particular phenotype may be the result of a perturbagen differentially regulating a distinct combination of genes. For instance, through its interaction with a particular transcription factor that, in turn, recognizes a particular DNA promoter sequence, a perturbagen may specifically elevate the expression of two or more target genes that act in concert to elicit a unique phenotype (e.g. viral resistance). One method of identifying such patterns induced by perturbagen agents is to utilize the recent technology of microarray analysis (see, for instance, Cumrnings C. A. and Relman D. A. (2000) “Using DNA Microarrays to Study Host-Microbe Interactions.” Emerg Infect Dis. 6(5):513-525.)

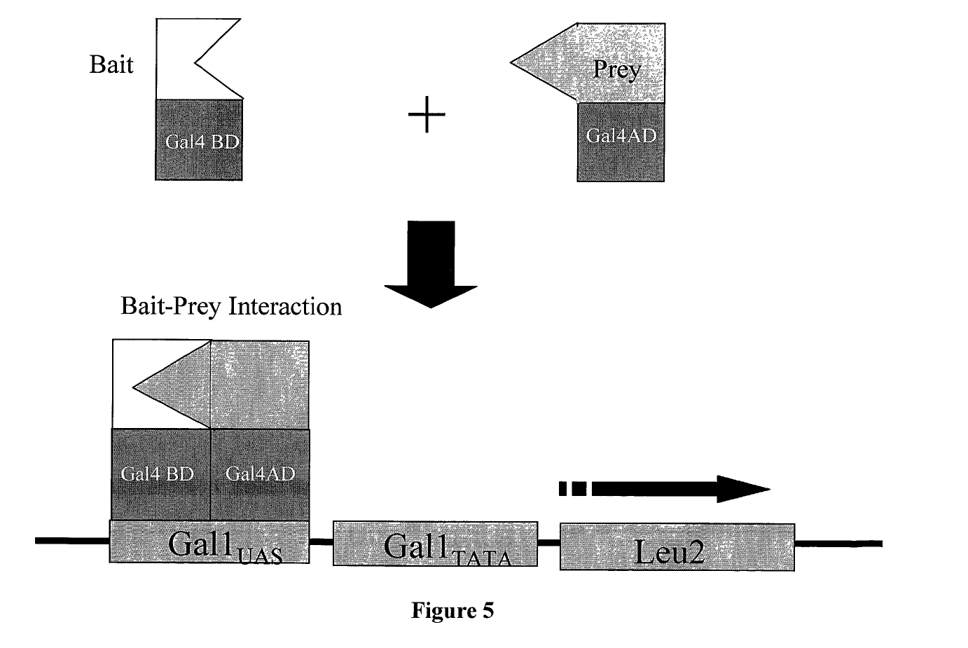

A preferred method of target identification involves application of variants of the standard two-hybrid technology. See, e.g., U.S. Pat. Ser. No. 09/193,759 and WO 00/29565 “Methods for validating polypeptide targets that correlate to cellular phenotypes”, the entire disclosures of which are incorporated by reference herein. Generally stated, the two-hybrid procedure is a quasi-genetic approach designed to detect binding events. This assay often is performed in yeast cells (although it can be adapted for use in mammalian and bacterial cells), and relies upon constructing two vectors; the first having an interaction probe or bait (that in this case, will be the perturbagen) that typically is fused to a DNA binding domain (“BD”) moiety, and a second vector having an interaction target or prey (a cDNA library derived from the host or from the viral pathogen, see, for example, Bryant, L. A. et al. (2000) “The human cytomegalovirus 86-kilodalton major immediate-early protein interacts physically and functionally with histone acetyltransferase P/CAF.” J Virol. 74(16):7230-7; Di Pasquale G and Stacey SN (1998) “Adeno-associated virus Rep78 protein interacts with protein kinase A and its homolog PRKX and inhibits CREB-dependent transcriptional activation” J. Virol 72(10):7916-25) that is typically fused to a DNA transcriptional moiety (the “activation domain” or “AD”). Neither of the two fusion proteins can, individually, induce transcription of the reporter gene. Yet when the bait and prey interact, the AD and BD moieties are brought into sufficient physical proximity to result in transcription of a reporter gene (e.g., the His3 gene or lacZ gene) located downstream of the bound complex (FIG. 5). Prey/bait interactions are then detected by identifying yeast cells that are expressing the reporter gene=13 e.g. which express lacZ or are able to grow in the absence of histidine.

A variety of yeast host strains known in the art are suitable for use for identifying targets of individual perturbagens. One of ordinary skill will appreciate that a number of factors may be considered in selecting suitable host strains, including but not limited to (1) whether the host cells can be mated to cells of opposite mating type (i.e., they are haploid), and (2) whether the host cells contain chromosomally integrated reporter constructs that can be used for selections or screens (e.g., His3 and LacZ). Although mating can be desirable in some embodiments, it is not strictly necessary for purposes of practicing the present invention. For example, the mating procedures can be eliminated by introducing the bait and prey constructs into a single yeast cell, whereupon the screens can be performed on the haploid cell.

Generally, either Gal4 strains or LexA host strains may be used with the appropriate reporter constructs. Representative examples include strains yVT 69, yVT 87, yVT96, yVT97, yVT98 and yVT99, yVT100, yVT360. Additionally, those of ordinary skill will appreciate that the host strains used in the present invention may be modified in other ways known to the art in order to optimize assay performance. For example, it may be desirable to modify the strains so that they contain alternative or additional reporter genes that respond to two-hybrid interactions.

The following host yeast strains are thus constructed to have the indicated characteristics:

YVT69: yVT69 (mat α, ura3-52, his3-200, ade2-101, trp1-901, leu2-3, 112, gal4A, met⁻, gal80Δ, URA3::GAL1_{UAS}-GAL1^{TATA}-lacZ) was obtained from Clontech (Y187).

YVT87: yVT87 (Mat-α ura3-52, his3-200, trp1-901 , LexA_{op(X6)}-LEU2-3, 112) was obtained from Clontech (EGY48).

YVT96: The starting strain was YM4271 (Liu, J. et al., 1993) MATa, ura3-52 his3-200 ade2-101 ade5 lys2-801 leu2-3, 112 trp1-901 tyr1-501 gal 4Δ gal80Δ ade5::hisG. YM4271 was converted to yVT96, MATa ura3-52 his3-200 ade 2-101 ade5 lys2::GAL2-URA3 leu2-3, 112 trp1-901 tyr1-501 gal4D gal80Δ ade5::hisG by homologous recombination of Reporter 1 to the LYS2 locus. The integration is confirmed by PCR.

YVT97: The starting strain is YM4271 (Liu, J. et al., 1993) MATa, ura3-52 his3-200 ade2-101 ade5 lys2-801 leu2-3, 112 trp1-901 tyr1-501 gal4Δ gal80Δ ade5::hisG. YM4271 will be converted to yVT97, MATα ura3-52 his3::GAL1 or GAL7-HIS3 ade2-101 ade5 lys2-801 leu2-3, 112 trp1-901 tyr1-501 gal4Δ gal80Δ ade5::hisG by the steps of (a) converting from MATa to MATα via transient expression of the HO endonuclease, Methods in Enzymology Vol. 194:132-146 (1991) and (b) integrating either of Reporters 3 or 4 at the HIS3 locus via homologous recombination. The integration is confirmed by PCR.

YVT98: The starting strain was EGY48 (Estojak, J. Et al., 1995) MATα, ura3 his3 trp1 leu2::LexAop(x6)-LEU2. EGY48 was converted to strain yVT98 MATα ura3 his3 trp1 leu2::lexAop(x6)-LEU2 lys2::lexAop(8× or 2×)-LacZ by homologous recombination of Reporter 6 into the LYS2 locus.

YVT99: The starting strain was EGY48 (Estojak, J. Et al., 1995) MATα, ura3 his3 trp1 leu2::LexAop(x6)-LEU2. EGY48 was converted to strain yVT99 MATa ura3 his3 trp1 leu2::lexAop(x6)-LEU2 lys2::lexAop(8× or 2×)-URA3 by homologous recombination of Reporter 2 into the LYS2 locus and by switching the mating type from MATα to MATa via transient expression of the HO endonuclease.

YVT100: The starting strain was YM4271 (Liu, J. et al., 1993) MATa, ura3-52 his3-200 ade2-101 ade5 lys2-801 leu2-3, 112 trp1-901 tyr1-501 gal4Δ gal80Δ ade5::hisG. YM4271 was converted to yVT100, MATa ura3-52 his3-200 ade2-101 ade5 lys2::lexAop(8× or 2×)-URA3 leu2-3, 112 trp1-901 tyr-501 gal4Δ gal80Δ ade5::hisG by homologous recombination of Reporter 2 to the LYS2 locus. The integration was confirmed by PCR.

YVT360: yVT360 (mat a, trp1-901, leu2-3, 112, ura3-52, his3-200, gal4Δ, gal80Δ, LYS2::GAL1_{UAS}-GAL1_{TATA}-HIS3, GAL2_{UAS}-GAL2_{TATA}-ADE2, URA3:MEL1_{UAS}-MEL1_{TATA}-lacZ) was obtained from Clontech (AH109).

Exemplary yeast-reporter strains are constructed using a variety of standard techniques. Many of the starting yeast strains already carry multiple mutations that lead to an auxotrophic phenotype (e.g. ura3-52, ade2-101). When necessary, reporter constructs can be integrated into the genome of the appropriate strain by homologous recombination. Successful integration can be confirmed by PCR. Alternatively, reporters may be maintained in the cells episomally.

The yeast two-hybrid reporter gene typically is fused to an upstream promoter region that is recognized by the BD, and is selected to provide a marker that facilitates screening. Examples include the lacZ gene fused to the Gal1 promoter region and the His3 yeast gene fused to Gal1 promoter region. A variety of yeast two-hybrid reporter constructs are suitable for use in the present invention. One of ordinary skill will appreciate that a number of factors may be considered in selecting suitable reporters, including whether (1) the reporter construct provides a rigorous selection (i.e., yeast cells die in the absence of a protein-protein or peptide-protein interaction between the bait and prey sequences), and/or (2) the reporter construct provides a convenient screen (e.g., the cells turn color when they harbor bait and prey sequences that interact). Examples of desirable reporters include (1) the Ura3 gene, which confers growth in the absence of uracil and death in the presence of 5-fluoroorotic acid (5-FOA); (2) the His3 gene, which permits growth in the absence of histidine; (3) the LacZ gene, which is monitored by a colorimetric assay in the presence/absence of beta-galactosidase substrates (e.g. X-gal); (4) the Leu2 gene, which confers growth in the absence of leucine; and (5) the Lys2 gene, which confers growth in the absence of lysine or, in the alternative, death in the presence of α-aminoadipic acid. These reporter genes may be placed under the transcriptional control of any one of a number of suitable cis-regulatory elements, including for example the Gal2 promoter, the Gal1 promoter, the Gal7 promoter, or the LexA operator sequences.

The following are exemplary, non-limiting examples of such reporter constructs.

Reporter 1—(pVT85): This reporter comprises the URA3 gene under the transcriptional control of the yeast Gal2 upstream activating sequence (UAS). In order to facilitate integration of this reporter into the yeast chromosome in place of the Lys2 coding region, the Gal2-Ura3 construct is flanked on the 5′ side by the 500 base pairs that lie immediately upstream of the coding region of the LYS2 gene and on the 3′ side by the 500 base pairs that lie immediately 3′ of the coding region of the LYS2 gene. The entire vector is also cloned into the yeast centromere containing vector pRS413 (Sikorski, R S and Hieter, P., Genetics 122(1):19-27 (1989) and can therefore be used episomally. This reporter is intended for use with a Gal4-based two-hybrid system, e.g., Fields, S. and Song, O., Nature 340:245-246 (1989).

Reporter 2—(pVT86): This reporter is identical to reporter #1 except that the GAL2 UAS sequences have been replaced with regulatory promoter sequences that contain eight LexA operator sequences (Ebina et al., 1983). The number of LexA operator sequences in this reporter may either be increased or decreased in order to obtain the optimal level of transcriptional regulation. This reporter is intended to be used within the general confines of the LexA-based interaction trap devised by Brent and Ptashne.

Reporter 3—(pVT87): This reporter is comprised of the yeast His3 gene under the transcriptional control of the yeast Gal1 upstream activating sequence (UAS). In order to facilitate integration of this reporter into the yeast chromosome in place of the His3 coding region the Gal1-His3 construct is flanked on the 5′ side by the 500 base pairs (bp) immediately upstream of the His3 coding region and on the 3′ side by the 500 bp immediately 3′ of the His3 coding region. The entire reporter is also cloned into the yeast centromere containing vector pRS415 and can therefore be used episomally. This reporter is intended for use with a Gal4-based two-hybrid system.

Reporter 4—(pVT88): This reporter is identical to Reporter 3 except that the His3 gene is under the transcriptional control of Gal7 UAS sequences rather than the Gal1 UAS. The reporter is used with a Gal4-based two-hybrid system.

Reporter 5—(pVT89): This reporter contains the bacterial LacZ gene under the transcriptional control of the Gal1 UAS. The entire reporter will be cloned into a yeast centromere-using vector, e.g., pRS413, and is used episomally.

Reporter 6—(pVT90): This reporter consists of the LacZ gene under the transcriptional control of eight LexA operator sequences. As for Reporter 2, the number of LexA operator sequences in this reporter may either be increased or decreased in order to obtain optimal levels of transcriptional regulation. Two features of this reporter facilitate integration of the reporter into the yeast chromosome in place of the Lys2 coding region. First, it is flanked on the 5′ side by the 500 base pairs that lie immediately upstream of the coding region of the Lys2 gene and on the 3′ side by the 500 base pairs that lie immediately 3′ of the coding region of the Lys2 gene. Second, the neomycin (NEO) resistance gene has been inserted between the 5′ Lys2 sequences and the LexA promoter sequences. This reporter is used in conjunction with a LexA-based interaction trap, e.g., Golemis, E. A., et al., (1996), “Interaction trap/two hybrid system to identify interacting proteins.” Current Protocols in Molecular Biology, Ausebel et al., eds., New York, John Wiley & Sons, Chap. 20.1.1-20.1.28.

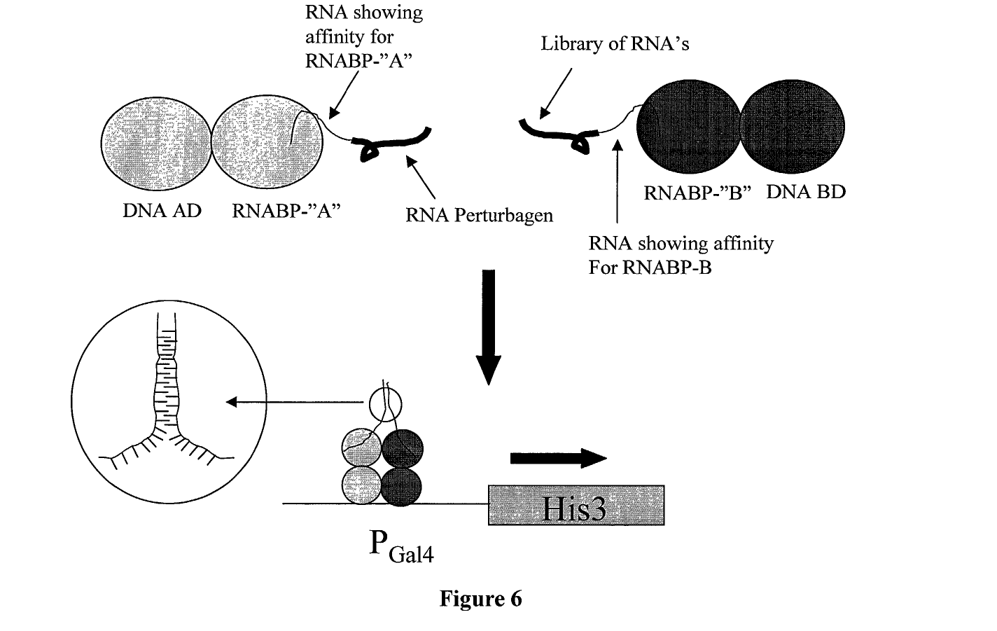

In other embodiments, perturbagen-induced phenotypes may be the result of RNA-RNA, RNA-polypeptide, polypeptide-DNA, or RNA-DNA interactions. In cases such as these, variations of the original two-hybrid theme may be applied to identify the target of the phenotypic probe. (See, for example, Li, J. J. and Herskowitz, I. (1993) Isolation of Orc6, a Component of the Yeast Origin Recognition Complex by a One-Hybrid System. Science, 262:1870-1874; Svinarchuk, F. et al. (1997) “Recruitent of transcription factors to the target site by triplex-forming oligonucleotides.” NAR 25: 3459-3464; Segupta, D. J. et al. (1999) “Identification of RNAs that bind to a specific protein using the yeast three-hybrid system.” RNA 5:596-601; Harada, K. et al. (1996) “Selection of RNA-binding peptides in vivo.” Nature 14;380(6570):175-9; SenGupta, D. J. et al. (1996) “A three-hybrid system to detect RNA protein interactions in vivo.” PNAS 93:8496-8501). For instance, if evidence exists that a perturbagen is acting as an anti-sense agent, it is necessary to construct a system where the association of the DNA binding domains and the transcriptional activation domains is dependent upon and RNA-RNA interaction. To accomplish such a screen, four unique vectors are created (FIG. 6). The first vector consists of the DNABP (e.g. GAL4 BD) described previously, linked to a specific RNA binding protein, arbitrarily called “RNABP-A” (e.g. the Rev responsive element RNA binding protein, RevM10, see Putz, U. et al. (1996) “A tri-hybrid system for the analysis and detection of RNA-protein interactions.” NAR 24:4838-4840). Vector #2 contains the transcriptional activation domain (e.g. GAL4 AD) linked to a second RNA binding protein (“RNABP-B”, e.g. the MS2 coat protein of the MS2 bacteriophage, see for example, SenGupta, D. J. et al. (1996) “A three hybrid system to detect RNA-protein interactions in vivo.” PNAS 93:8496-8501). The third vector encodes an RNA molecule that is recognized by RNABP-A (e.g. the RRE sequence, Zapp, M. L. and Green M. R/“Sequence-specific RNA binding by the HIV-1 Rev protein (1989) Nature, 32:714-716) fused to a sequence encoding the RNA perturbagen, while the final vector encodes a fourth hybrid, the RNA sequence recognized by RNABP-B (e.g. the 21 base nucleotide RNA stem-loop structure of MS2, see Uhlenbeck, O. C. et. al. (1983) “Interaction of R17 coat protein with its RNA binding site for translational repression.” J. Biomol Struct. Dyn. 1, 539-552) linked to a library of expressed sequences (e.g. a library of mRNA molecules). When all four vectors are stably maintained in a yeast cell containing the necessary reporter construct(s) (e.g. P_{GAL4}-LACZ), the cellular target RNA molecule of any given RNA perturbagen can be identified.

Target sequences or fragments thereof can vary greatly in size. Some target fragments can be as small as ten amino acids in length. Alternatively, target sequences can be greater than 10 amino acids but less than thirty amino acids in length. Still other targets can be greater than thirty amino acids in length but shorter than 60 amino acids in length. Still other targets are cellular proteins or subunits or domains therein of more than 60 amino acids in length. Still other targets are cellular proteins or subunits or domains there of more than 60 amino acids in length. Still other targets are cellular proteins or subunits or domains there of more than 60 amino acids in length. In addition, for reasons described previously, the sequences encoding targets can vary greatly due to allelic variation, duplications and closely related gene family members. That said, the invention also encompasses variants of said targets. A preferred target variant is one which has at least about 80%, alternatively at least about 90%, and in another alternative at least about 95% amino acid sequence identity to the original target amino acid sequence and which contains at least one functional or structural characteristic of the original target.

L. Modes of Action

Several experiments can be performed to determine the timing and/or mode of action of a given perturbagen. For instance, viral RNA can be labeled with radioactive isotopes to assess whether the perturbagen prevents the virus from injecting its genome into the cell. Similarly, experiments based on neutral-red sensitivity can be performed to determine whether the perturbagen alters the rate of viral uncoating (see, for example, Fox, P. M. et al. (1986) “Prevention of Rhinovirus and Poliovirus Uncoating by WIN 51711, a New Antiviral Drug.” Antimicrobial Agents and Chemotherapy 30:110-116). Still additional clues to the mode of action of a perturbagen can be obtained by taking a genetic approach. For instance, if a hypothetical perturbagen acts by inhibiting the catalytic activity of a particular viral protease, it may be possible to isolate one or more viral mutants that are resistant to the perturbagen. By sequencing the viral genome of such mutants, it is possible to identify which gene is responsible for the alteration in perturbagen sensitivity (see, for instance, Heinz, B. A. and Vance, L. M. (1995) “The antiviral compound enviroxime targets the 3A coding region of rhinovirus and poliovirus.” J. Virol. 69(7):4189-97).

Another method to understanding the mode of action of an antiviral perturbagen focuses on examining the expression of heat shock proteins (specifically hsp70) in HeLa cells. Previous clinical studies have shown that patients with naturally acquired or experimental-induced colds benefited from brief hyperthermic treatment (HT). This finding, when combined with the observation that various hsp-inducers (e.g. PGA1, and Δ¹²-PGJ2) were also effective in inhibiting RV replication, supported the notion that hsp's mediated the antiviral effects induced by HT (see, for example, Santoro M. G. (1994) “Heat shock proteins and virus replication: hsp70s as mediators of the antiviral effects of prostaglandins.” Experientia 50(11-12):1039-47; Conti, C. (1999) “Antiviral Effect of Hyperthermic Treatment in Rhinoviral Infection.” Antimicrobial Agents and Chemotherapy 43:822-29)). Experiments can be performed to determine whether the action of a perturbagen is mediated by changes in heat shock protein levels or modifications to heat shock proteins (e.g. phosphorylation). For instance, cytosolic proteins purified from HeLa cells cultured under various conditions can be analyzed on a Western Blot with antibodies that recognize hsp70 and to determine whether the perturbagen alters the level of expression of the heat-shock protein. Alternatively, experiments can be designed using radiolabeled isotopes of phosphate to assess the level of phosphorylation of various heat-shock proteins present in HeLa cells (see, for example, Nakatsue, T. et al. (1998) “Acute infection of Sindbis virus induces phosphorylation and intracellular translocation of small heat shock protein HSP27 and activation of p38 MAP kinase signaling pathway.” Biochem Biophys Res Commun 9;253(1):59-64).

M. Databases

The compositions, relations and phenotypic effects yielded by the methodology described herein may advantageously be placed into or stored in a variety of databases. As one example, a database may include information about one or more targets identified by the methods herein, including for example sequence information, motif information, structural information and/or homology information. The database may optionally contain such information regarding perturbagen agents, and may correlate the perturbagen information to corresponding target information. Further helpful database aspects may include information regarding, e.g., variants or fragments of the above. The database may also correlate the indexed compounds to, e.g., immunoprecipitation data, further yeast n-hybrid interaction data, genotypic data (e.g., identification of disrupted genes or gene variants), and with a variety phenotypic data. Such databases are preferably electronic, and may additionally be combined with a search tool so that the database is searchable.

N. Production of antibodies

An additional embodiment of the invention includes antibodies that recognize the perturbagen itself, cellular targets of the perturbagen, or one or more epitopes of the foregoing. Such reagents may include, but are not limited to, polyclonal, monoclonal, humanized, chimeric, and single chain antibodies, Fab fragments, F(ab′)₂ fragments, fragments produced by a Fab expression library, anti-idiotypic (anti-Id) antibodies, and epitope-binding fragements of any of the above. Antibodies directed against perturbagens or cellular targets may be useful for a variety of purposes including i) therapeutics, ii) diagnostic assays, iii) cytoimmunology, iv) target identification, and v) purification.

For the production of antibodies, various hosts including goats, rabbits, rats, mice, humans and others may be immunized by injection with a perturbagen, target or any fragment thereof which has immunogenic properties. Depending on the host species, various adjuvants may be used to increase immunological response. Such adjuvants include, but are not limited to Freund's (complete and incomplete), mineral gels such as aluminum hydroxide, and surface-active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

Polyclonal antibodies are heterogeneous populations of antibody molecules derived from the sera of animals immunized with an antigen, such as a given perturbagen, target, or an antigenic functional derivative thereof. For the production of polyclonal antibodies, host animals such as those described above, may be immunized by injection with gene product supplemented with adjuvants as also described above.

Monoclonal antibodies that recognize perturbagens may be prepared using any technique that provides for the production of antibody molecules by continuous cell lines in culture. These include, but are not limited to, the hybridoma technique, the human B-cell hybridoma technique, and the EBV hybridoma technique. (see, for example, Kohler, G. et al. (1975) “Continuous cultures of fused cells secreting antibody of predefined specificity.” Nature 256:495-497; Kozbor, D. et al (1985) “Specific immunoglobulin production and enhanced tumorigenicity following ascites growth of human hybridomas.” J. Immunol. Methods 81:31-42; Cote, R. J. et al. (1983) PNAS 80:2026-2030; and Cole, S. P. et al. (1984) “Generation of human monoclonal antibodies reactive with cellular antigens” Mol. Cell Biol. 62:109-120).

In addition, one may use techniques developed for the production of chimeric antibodies, such as the splicing of mouse antibody genes to human antibody genes to obtain a molecule with appropriate antigen specificity and biological activity. See, e.g., Morrison, S. L. et al. (1984) “Chimeric human antibody molecules: mouse antigen-binding domains with human constant region domains.” PNAS 81:6851-6855); Neuberger, M. S. et al. (1984) “Recombinant antibodies possessing novel effector functions.” Nature 312:604-608; and Takeda, S. et al. (1985) “Construction of chimeric processed immunoglobulin genes containing mouse variable and human constant region sequences.” Nature 314:452-454). Alternatively, techniques described for the production of single chain antibodies may be adapted, using methods known in the art, to produce perturbagen-specific antibodies (see, e.g. Burton, D. R. (1991) “A large array of human monoclonal antibodies to type I human immunodeficiency virus from combinatorial libraries of asymptomatic seropositive individuals.” PNAS 88:10134-10137).

Antibodies may also be produced by inducing in vivo production in the lymphocyte population or by screening immunoglobulin libraries or panels of highly specific binding reagents as disclosed in the literature. (see, for example, Orlandi, R. et al. (1989) “Cloning immunoglobulin variable domains for expression by the polymerase chain reaction.” PNAS 86:3833-3837; Winter, G. et al. (1991) “Man-made antibodies.” Nature 349: 293-299).

Antibody fragments that contain specific binding sites for perturbagens may also be generated. For example, such fragments include, but are not limited to F(ab′)₂ fragments produced by pepsin digesting of the antibody molecule and Fab fragments generated by reducing the disulfide bridges of the F(ab′)₂ fragments. Alternatively, Fab expression libraries may be constructed to allow rapid and easy identification of monclonal Fab fragments with the desired specificity. (See, for example, Huse, W. D. et al. (1989) “Generation of a large combinatorial library of the immunoglobulin repertoire in phage lambda.” Science 246:1275-1281).

O. Screening Assays

The agents of the invention can be used to screen for drugs or compounds (small molecules) that mimic, or modulate the activity or expression of said phenotypic probes.

Like the perturbagen itself, such compounds may be used to treat disorders characterized by viral infection. Thus, the invention provides a method for identifying modulators, i.e. candidate or test compounds or agents (e.g. peptidomimetics, small molecules or other drugs) that bind to the agent or its target, and have a stimulatory or inhibitory effect on the pathway(s) affected by said agent.

In vitro systems may be designed to identify compounds capable of binding, e.g., a viral target gene product. Such compounds may include, but are not limited to, peptides made of D-andlor L-configuration amino acids (in, for example, the form of random peptide libraries; (see e.g., Lam, et al., Nature, 354:82-4 (1991)), phosphopeptides (in, for example, the form of random or partially degenerate, directed phosphopeptide libraries; see, e.g., Songyang, et al., Cell, 72:767-78 (1993)), antibodies, and small organic or inorganic molecules. Compounds identified may be useful, for example, in modulating the activity of viral target gene proteins, preferably mutant proteins; elaborating the biological function of the viral target gene protein; or screening for compounds that disrupt normal viral target gene interactions or themselves disrupt such interactions.

In one embodiment, the invention provides libraries of test compounds. The test compounds of the present invention can be obtained using any of the numerous approaches in combinatorial library methods known in the art, including: biological libraries, spatially addressable parallel solid phase or solution phase libraries; synthetic library methods requiring deconvolution; the one-bead one-compound library method; and synthetic library methods using affinity chromatography selection. The biological library approach is exemplified by peptide libraries, while the other four approaches are applicable to peptide, non-peptide oligomer or small molecule libraries of compounds (Lam, K. S. (1997) “Application of combinatorial library methods in cancer research and drug discovery.” Anticancer Drug Des. 12:145).

Methods for the synthesis of molecular libraries can be found in the art, for example, in (i) De Witt, S. H. et al. (1993) “Diversomers: an approach to nonpeptide, nonoligomeric chemical diversity.” PNAS 90:6909, (ii) Erb, E. et al. (1994) “Recursive deconvolution of combinatorial chemical libraries.” PNAS 91:11422, (iii) Zuckermann, R. N. et al. (1994) “Discovery of nanomolar ligands for 7-transmembrane G-protein-coupled receptors from a diverse N-(substituted)glycine peptoid library.” J. Med Chem. 37: 2678 and (iv) Cho, C. Y. et al. (1993) “An unnatural biopolymer.” Science 261:1303. Libraries of compounds may be presented in i) solution (e.g. Houghten, R. A. (1992) “The use of synthetic peptide combinatorial libraries for the identification of bioactive peptides.” BioTechniques 13:412) ii) on beads (Lam, K. S. (1991) “A new type of synthetic peptide library for identifying ligand-binding activity.” Nature 354:82), iii) chips (Fodor, S. P. (1993) “Multiplexed biochemical assays with biological chips.” Nature 364:555), iv) bacteria (U.S. Pat. No. 5,223,409), v) spores U.S. Pat. Nos. 5,571,698, 5,403,484, and 5,223,409), vi) plasmids (Cull, M. G. et al. (1992) “Screening for receptor ligands using large libraries of peptides linked to the C terminus of the lac repressor.” PNAS 89:1865) or vii) phage (Scott, J. K. and Smith, G. P. (1990) “Searching for peptide ligands with an epitope library.” Science 249: 386)

There are several methods for identifying small molecule compounds that mimic the action of the phenotypic probes. In one approach, an assay may be devised to directly identify agents that bind to, e.g., an RV-related target protein. Such direct binding assays generally involve preparing a reaction mixture of the RV-related target protein and the test compound under conditions and for a time sufficient to allow the two components to interact and bind, thus forming a complex that can be removed and/or detected in the reaction mixture. These assays can be conducted in a variety of ways. For example, one method to conduct such an assay would involve anchoring the RV-related target protein or the test substance onto a solid phase and detecting target protein/test substance complexes anchored on the solid phase at the end of the reaction. In one embodiment of such a method, the RV-related target protein may be anchored onto a solid surface, and the test compound, which is not anchored, may be labeled, either directly or indirectly.

In practice, microtitre plates are conveniently utilized. The anchored component may be immobilized by non-covalent or covalent attachments. Non-covalent attachment may be accomplished simply by coating the solid surface with a solution of the protein and drying. Alternatively, an immobilized antibody, preferably a monoclonal antibody, specific for the protein may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for an RV-related gene product or the test compound to anchor any complexes formed in solution, and a labeled antibody specific for the other component of the possible complex to detect anchored complexes.

Compounds that are shown to bind to a particular RV-related gene product through one of the methods described above can be further tested for their ability to elicit a biochemical response from the RV-related gene protein. Agonists, antagonists and/or inhibitors of the expression product can be identified utilizing assays well known in the art.

In another approach, perturbagen/target pairs are used to identify small molecule mimetics in a displacement assay format. Such assays can be based upon a variety of technologies including, but not limited to i) ELISAs (see, for example, Rice, J. W. et al. (1996) “Development of a high volume screen to identify inhibitors of endothelial cell activation.” Anal Biochem 241(2):254-9), ii) scintillation proximity assays (see, for example, Lerner, C. G. and Saiki, A. Y. C. (1996) “Scintillation proximity assay for human DNA topoisomerase I using recombinant biotinyl-fusion protein produced in baculovirus-infected insect cells.” Anal Biochem 240(2): 185-96) or iii) time-resolved fluorescence resonance energy transfer-based technology (see, for example, Fernandes, P. B. (1998) “Technological advances in high-throughput screening.” Curr Opin Chem Biol 2(5):597-603; Hemmilä, “Time-resolved fluorometry—advantages and potentials in high throughput screening assays.” “High Throughput Screening”, J. Devlin (ed.). Marcel Dekker Inc, New York, pp. 361-76 (1997)). Two non-limiting examples of such assays, one homogeneous, LANCE™ (Stenroos, K. et al. (1997) “Homogeneous time resolved fluo-rescence energy transfer assay (LANCE) for the determination of IL-2-IL-2 receptor interaction.” Abstract of Papers Presented at the 3rd Annual Conference of the Society for Biomolecular Screening, Sep., California), and one heterogeneous, DELFIA™ (MacGregor, I. et al. (1999) “Application of a time-resolved fluoroimmunoassay for the analysis of normal prion protein in human blood and its components.” Vox Sang 77(2):88-96; Jensen, P. E. et al. (1998) “A europium fluoroimmunoassay for measuring peptide binding to MHC class I molecules.” J. Immunol. Methods 215: 71-80; Takeuchi, T. et al. (1995) “Nonisotopic receptor assay for benzodiazepine drugs using time-resolved fluorometry.” Anal. Chem. 67: 2655-8.) are described as follows.

1. Lance™: Homogeneous Assay

To identify small molecules capable of disrupting the interaction between the perturbagen and its target, assays are designed to utilize the LANCE™ technology (commercially available from E. G. & G. Wallac.). LANCE™ is a homogeneous assay that is performed in solution and requires no wash steps to separate bound and unbound label. Briefly, the target is produced in large quantities and labeled with a lanthanide chelate (i.e. a fluorescent donor such as a Europium, (Eu) or Terbium (Tb) chelate). Concomitantly, the perturbagen is labeled with one of several fluorescent “acceptor” moieties that can be excited by the emissions of the donor molecule (e.g. allophycocyanin (APC) or rhodamine Rh, respectively). Most preferably, 1) the modification of either the perturbagen or the target is not detrimental to the interaction between the two interacting molecules being studied and 2) the distance separating the donor and acceptor moieties when the perturbagen and the target are associated, is sufficiently close to permit FRET (typically 30-100 Angstroms). As an alternative to direct labeling of the perturbagen, monoclonal antibodies directed against the perturbagen can be labeled with Eu, thus allowing small molecule displacement assays to take place via indirect labeling procedures.

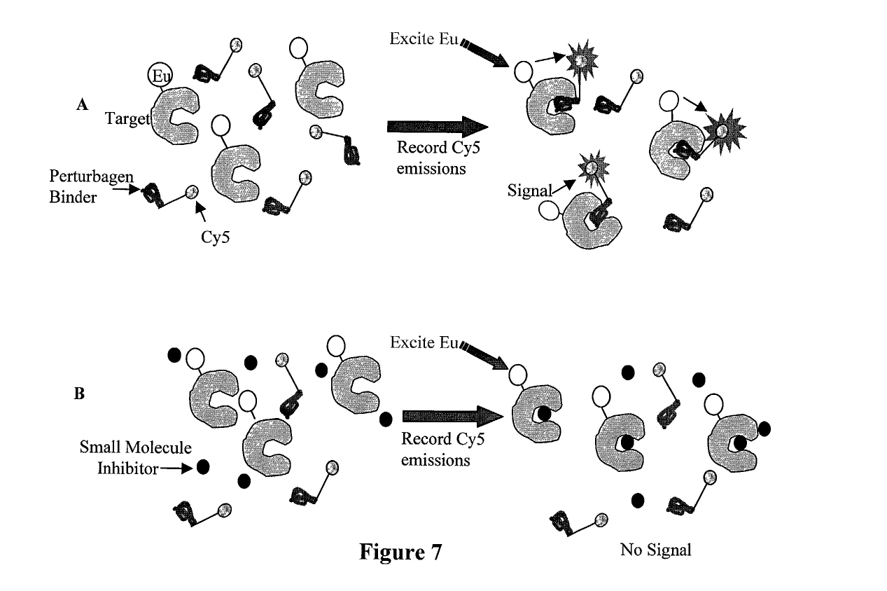

To identify small molecules capable of disrupting the interaction between the perturbagen and its target, the two labeled components are alliquoted into wells (1536 well format) at previously set, optimized conditions that will ensure 50% binding (FIG. 7). Subsequently, each well is then exposed to one or more members of a large chemical combinatorial library and time-resolved measurements are taken using a Wallac 1420 Victor multilabel counter or equivalent fluoremeter. In wells that contain a small molecule that interferes with the interaction between the perturbagen and its target, the distance separating the donor and acceptor molecules is increased. As a result of this dissociation or displacement, the ability of the Eu emissions to excite the acceptor is compromised and the total fluorescence emitted by the acceptor is decreased.

2. DELFIA™: Heterogeneous Assay

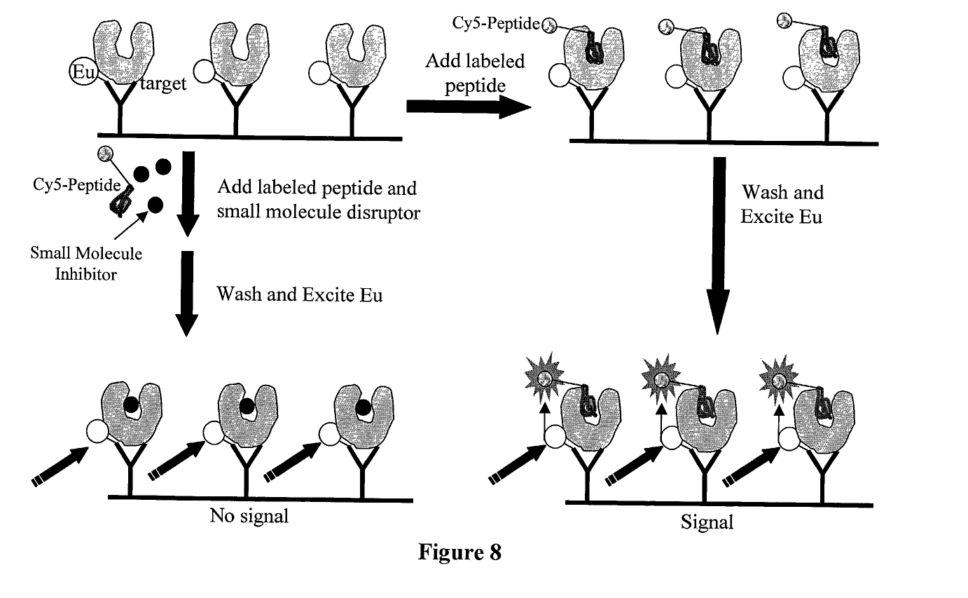

Several variations of a heterogeneous assay (DELFIA™) using an immobilized substrate can be used as an alternative to LANCE™. In one non-limiting example, the target is immobilized to a solid support using a monoclonal antibody that has been labeled with Eu (FIG. 8). Subsequent addition and binding of a rhodamine labeled perturbagen in the presence or absence of a candidate small organic displacement molecule is followed by several wash steps to remove unbound material. TR-FRET is then performed by exciting Eu and measuring the levels of Rh emissions. As an alternative to this procedure, the target is immobilized to the solid support using an unlabeled monoclonal antibody. Subsequently, an Eu-labeled perturbagen (+/− a candidate small organic displacement molecule) is added to each well and allowed to equilibrate, followed by a washing procedure to eliminate unbound Eu-labeled material. Once the well has been cleared of all unbound material, the bound Eu-perturbagen molecules are released and excited in the presence of commercially available enhancement solutions (DELFIA™ Enhancement Solutions, Wallac). By comparing the levels of emissions in wells that contain members of the molecule library with standardized controls, small molecules that disrupt the interaction between the perturbagen and its target are identified.

P. Therapeutic Uses

Anti-rhinoviral agents can be used against over a hundred serotypes of rhinovirus and may be effective fighting other, closely related, infectious agents belonging to the picornaviridae family. For that reason, in one embodiment, perturbagens, fragments or derivatives of a perturbagen, small molecule mimetics of a perturbagen, sequences encoding perturbagens, sequences that can hybridize to perturbagen encoding sequences, targets of the perturbagen, or agents that bind said target (e.g. antibodies) or portions thereof, may be utilized to treat or prevent disorders that result from viral infections by the picomaviridae class. Thus, for example, polypeptides or RNA molecules described herein can be used prevent, combat, or minimize the clinical symptoms evoked by i) members of the rhinovirus genus including human rhinovirus IA-100, 1B, and “Hanks” as well as bovine rhinoviruses 1, 2, and 3; ii) members of the enterovirus genus including human polio viruses 1, 2, and 3, human coxsackieviruses A1-22, 24, B1-6, human echoviruses 1-7, 9, 11-27, 29-34, 68-71, vilyuisk virus, simian enteroviruses 1-18, bovine enteroviruses 1 and 2, and porcine enteroviruses 1-8; iii) apthovirus including Foot and mouth disease virus 1-7 (serotypes A, C, O, SAT-1, 2, 3, and Asian-1); iv) cardioviruses including encephalomyocarditis (EMC) virus and Theiler's murine encephalomyelitis (TME); v) hepatoviruses including human hepatitis virus A, and vi) unassigned viruses including equine rhinoviruses 1, and 2, cricket paralysis virus, Drosophila C virus as well as other related genuses.

Viral-induced ailments can be treated with the perturbagen directly, for example by administering a therapeutically effective dose of a proteinaceous agent intravenously or by other peptide delivery techniques known to the art. A therapeutically effective dose of a pharmaceutical composition comprising a substantially purified perturbagen, or a fragment thereof, or a small molecule mimetic, optionally in conjunction with a suitable pharmaceutical carrier, may be administered to a subject to treat or prevent a viral disorder. A “therapeutically effective” dose refers to that amount of the compound sufficient to result in amelioration of symptoms of the disease. A “pharmaceutical carrier” includes any and all solvents, dispersion media, coatings, antibacterial and antifungal agents, and the like, compatible with pharmaceutical administration. The use of such media and agents for pharmaceutically active substances is well known in the art. Except insofar as any conventional media or agent is incompatible with the active compound, use thereof in the compositions is contemplated.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Pharmaceutical compositions of the invention are formulated to be compatible with intended routes of delivery. Examples of routes of administration include parenteral e.g. intravenous, intradermal, subcutaneous, oral, inhalation, transdermal, topical, transmucosal, and rectal administration. Solutions or suspensions used for parenteral, intradermal, or subcutaneous application can include the following components: a sterile diluent, such as water for injection, saline solution, fixed oils, polyethylene, glycols, glycerine, propylene glycol, or other synthetic solvents, antibacterial agents such as benzyl alcohol or methyl parabens, antioxidants such as ascorbic acid or sodium bisulfite, chelating agents such as ethylenediaminetetraacetic acid, buffers such as acetates, citrates, or phosphates and agents for the adjustment of tonicity such as sodium chloride or dextrose.

Pharmaceutical compositions suitable for injectable use include aqueous solutions (where water-soluble) or dispersions and sterile powders for the extemporaneous preparation of sterile injectable solutions or dispersions. For intravenous administration, suitable carriers include physiological saline, bacteriostatic water Cremophor EL™ (BASF; Parsippany, N.J.) or phosphate buffered saline (PBS). In all cases the composition must be sterile and should be fluid to the extent that easy syringability exists. Oral compositions can also be prepared using any of the following ingredients, or compounds of a similar nature: a binder such as microcrystalline cellulose, gum tragacanth, or gelatin; an excipient such as starch or lactose, disintegrating agent such as alginic acid, Primogel, or corn starch; a lubricant such as magnesium stearate, a glidant such as colloidal silicon dioxide, a sweetening agent such as sucrose or saccharin, or a flavoring agent such as peppermint or orange flavoring. For administration by inhalation, the compounds are delivered in the form of an aerosol spray from a pressurized container or dispenser that contains a suitable propellant. Systemic administration can also be by transmucosal or transdermal means. For these methods of administration, penetrants appropriate to the barrier to be permeated are used in the formulation. Such penetrants are generally known in the art and include, for example, bile salts and fusidic acid derivatives. Transmucosal administration can also be accomplished through the use of nasal sprays and suppositories. For transdermal administration, the active compounds are formulated into ointments, salves, gels, or creams as generally known in the art.

In one embodiment, the active compounds are prepared with carriers that will protect the compound against rapid elimination from the body, such as a controlled microencapsulated delivery system. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to specific cell surface epitopes) can also be used as pharmaceutically acceptable carriers. These can be prepared according to methods known to those skilled in the art, for example, as described in U.S. Pat. No. 4,522,811.

Alternatively, such therapeutics can be administered indirectly, for example by gene therapy utilizing a gene or RNA sequence encoding a perturbagen, viral infection-related target, or variant or fragment of the foregoing. For example, a vector capable of expressing a perturbagen or target, or a fragment or derivative thereof, may be administered to a subject to treat or prevent a disease. Expression vectors including, but not limited to, those derived from retroviruses, adenoviruses, adeno-associated viruses, or herpes or vaccinia viruses or from various bacterial plasmids, may be used for delivery of nucleotide sequences to the targeted organ, tissue, or cell population (see, for example, Carter, P. J. and Samulski, R. J. (2000) “Adeno-associated viral vectors as gene delivery vehicles.” Int J Mol Med. 6(1):17-27; Palu, G. et al. (2000) “Progress with retroviral gene vectors.” Rev Med Virol. 10(3):185-202; Wu, N. and Ataai, M. M. (2000) “Production of viral vectors for gene therapy applications.” Curr Opin Biotechnol. 11(2):205-8). Gene therapy vectors can be delivered to a subject by, for example, intravenous injection, local administration (U.S. Pat. No. 5,328,470) or by stereotactic injection (see, for example, Chen, S. H. et al. (1994) “Gene therapy for brain tumors: regression of experimental gliomas by adenovirus-mediated gene transfer in vivo.” PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

Q. Antisense, Ribozyme and Antibody Therapeutics

Other agents that may be used as therapeutics include any relevant target genes, associated expression product and functional fragments thereof. Additionally, agents that reduce or inhibit mutant target gene activity may be used to ameliorate disease symptoms. Such agents include antisense, ribozyme, and triple helix molecules. Techniques for the production and use of such molecules are well known to those of skill in the art.

Anti-sense RNA and DNA molecules act to directly block the translation of mRNA by hybridizing to targeted MnRNA and preventing protein translation. With respect to antisense DNA, oligodeoxyribonucleotides derived from the translation initiation site, e.g., between the −10 and +10 regions of the viral target gene nucleotide sequence of interest, are preferred.

Ribozymes are enzymatic RNA molecules capable of catalyzing the specific cleavage of RNA. The mechanism of ribozyme action involves sequence-specific hybridization of the ribozyme molecule to complementary target RNA, followed by an endonucleolytic cleavage. The composition of ribozyme molecules must include one or more sequences complementary to a target gene mRNA, and must include the well known catalytic sequence responsible for mRNA cleavage. For this sequence, see U.S. Pat. No. 5,093,246, which is incorporated by reference herein in its entirety.

Specific ribozyme cleavage sites within any potential RNA target are initially identified by scanning the molecule of interest for ribozyme cleavage sites that include the following sequences, GUA, GUU and GUC. Once identified, short RNA sequences of between 15 and 20 ribonucleotides corresponding to the region of the target gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays.

Nucleic acid molecules to be used in triple helix formation for the inhibition of transcription should be single stranded and composed of deoxyribonucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

It is possible that the antisense, ribozyme, and/or triple helix molecules described herein may reduce or inhibit the transcription (triple helix) and/or translation (antisense, ribozyme) of mRNA produced by both normal and mutant target gene alleles. In order to ensure that substantially normal levels of target gene activity are maintained, nucleic acid molecules that encode and express target gene polypeptides exhibiting normal activity may be introduced into cells that do not contain sequences susceptible to whatever antisense, ribozyme, or triple helix treatments are being utilized. Alternatively, it may be preferable to coadminister normal target gene protein into the cell or tissue in order to maintain the requisite level of cellular or tissue target gene activity.

Anti-sense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

Antibodies that are both specific for target gene protein, and in particular, mutant gene protein, and interfere with its activity may be used to inhibit mutant target gene function. Such antibodies may be generated against the proteins themselves or against peptides corresponding to portions of the proteins using standard techniques known in the art and as also described herein. Such antibodies include but are not limited to polyclonal, monoclonal, Fab fragments, single chain antibodies, chimeric antibodies, etc.

In instances where a target gene protein is intracellular and whole antibodies are used, internalizing antibodies may be preferred. However, lipofectin liposomes may be used to deliver the antibody or a fragment of the Fab region that binds to the target gene epitope into cells. Where fragments of the antibody are used, the smallest inhibitory fragment that binds to the target or expanded target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (see, e.g., Creighton, Proteins: Structures and Molecular Principles (1984) W. H. Freeman, New York 1983, supra; and Sambrook, et al., 1989, supra). Alternatively, single chain neutralizing antibodies that bind to intracellular target gene epitopes may also be administered. Such single chain antibodies may be administered, for example, by expressing nucleotide sequences encoding singlechain antibodies within the target cell population by utilizing, for example, techniques such as those described in Marasco, et al., Proc. Natl. Acad. Sci. USA, 90:7889-93 (1993).

R. Diagnostic Uses

The polynucleotides, polypeptides, variants, targets and antibodies to any one of these molecules can, in addition to previously mentioned therapeutic applications, be used in one or more of the following methods: 1) detection assays (e.g. chromosomal mapping, tissue typing, forensic biology, viral serotyping), and 2) predictive medicine (e.g. diagnostic or prognostic assays, pharmacogenomics and monitoring clinical trials). Thus, for example, agents may be used to detect a specific mRNA or gene (e.g. in a biological sample) for a genetic lesion. Alternatively, agents may be used to identify a particular serotype or sub-serotype of a given infectious agent. Similarly, agents described herein may be applied to the field of predictive medicine in which diagnostic assays or prognostic assays, pharmacogenomics, and monitoring clinical trials are used for predictive purposes to thereby treat an individual prophylactically.

Accordingly, one aspect of the present invention relates to diagnostic assays for determining expression of a polypeptide or nucleic acid of the invention and or activity of said agent of the invention, in the context of a biological sample to thereby determine whether an individual is afflicted with a disease or disorder, or is at risk of developing a disorder, associated with aberrant expression or activity of a polypeptide or polynucleotide of the invention.

Alternatively, the invention provides methods for detecting expression of a nucleic acid or polypeptide of the invention or activity of a polypeptide or polynucleotide of the invention in an individual to thereby select appropriate therapeutic or prophylactic agents for that individual (referred to herein as “pharmacogenomics”). Pharmoacogenomics allows for the selection of agents (e.g. drugs) for therapeutic or prophylactic treatment of an individual based on the genotype of the individual (e.g. the genotype of the individual examined to determine the ability of the individual to respond to a particular agent). Still another aspect of the invention pertains to monitoring the influence of agents (e.g. drugs or other compounds) on the expression or activity of a polypeptide or polynucleotide of the invention in clinical trials.

1. Detection Assays

Portions or fragments of the polynucleotide sequences of the invention can be used in numerous ways as polynucleotide reagents. For example, these sequences can be used to i) map their respective genes on a chromosome and, thus, locate gene regions associated with genetic diseases; ii) identify an individual from a minute biological sample (tissue typing); and iii) aid in forensic identification of biological samples.

a. Gene and Chromosome Mapping

Once the sequence (or portion of a sequence) of a gene has been isolated, this sequence can be used to identify the entire gene, analyze the gene for homology to other sequences (i.e., identify it as a member of a gene family such as EGF receptor family) and then map the location of the gene on a chromosome. Accordingly, nucleic acid molecules described herein or fragments thereof, can be used to map the location of the gene on a chromosome. The mapping of the sequences to chromosomes is an important first step in correlating these sequences with genes associated with disease.

Briefly, genes can be mapped to chromosomes by preparing PCR primers from the sequence of a gene of the invention. These primers can then be used for PCR screening of somatic cell hybrids containing individual chromosomes. Only those hybrids containing the human gene corresponding to the gene sequences will yield an amplified fragment (For review of this technique se D'Eustachio, P. and Ruddle, F. H. (1983) “Somatic cell genetics and gene families.” Science 220:919-924). Alternative methods of mapping a gene to its chromosome include in situ hybridization (see, for example, Fan, Y. S. et al. (1990) “Mapping small DNA sequences by fluorescence in situ hybridization directly on banded metaphase chromosomes.” PNAS 87:6223-27), pre-screening with labeled flow sorted chromosomes (CITE), and pre-selection by hybridization to chromosome specific cDNA libraries. Furthermore, fluorescence in situ hybridization (FISH) of a DNA sequence to a metaphase chromosome spread can further be used to provide a precise chromosomal location in one step (see “Human Chromosomes: A Manual of Basic Techniques”, Pergamon Press, New York, 1988). Lastly, with the completion (in the not-to-distant future) of the sequencing of the human genome, chromosome mapping will very quickly switch from elaborate, hands-on methods of mapping genes, to simple database searches

Once the sequence (or portion of a sequence) of a gene has been isolated, these agents can be used to assess the intactness or functionality of a particular gene. Comparison of affected and unaffected individuals can begin with looking for structural alterations in the chromosomes such as deletions, inversions, or translocations that are based on that DNA sequence. Once this is accomplished, the physical position of the sequence on the chromosome can be correlated with genetic data map. (such data are found, for example in McKusick, V. “Mendialian Inheritance in Man” available on-line through John Hopkins University Welch Medical Library). The relationship between genes and disease, mapped to the same chromosomal region can then be identified through linkage analysis (co-inheritance of physically adjacent genes), described in e.g. Egeland, J. A. et al. (1987) “Bipolar affective disorders linked to DNA markers on chromosome 11.” Nature , 325:783-787). Alternatively, polynucleotide sequences can be used as probes in Southern Blot analysis to identify alterations in the organization of the gene of interest and surrounding regions. Ultimately, complete sequencing of genes from several individuals can be performed to confirm the presence of a mutation and to distinguish mutations from polymorphisms. If a specific mutation is observed in some or all individuals affected by a particular disease, but not in any unaffected individuals, then the mutation is likely to be the causative agent of the particular disease.

b. Tissue Typing

The nucleic acid sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. The sequences of the present invention are useful as additional DNA markers for RFLP mapping (described in U.S. Pat. No. 5,272,057).

Furthermore the sequences of the present invention can be used to determine the actual base-by-base DNA sequence of selected portions of an individual's genome. Thus, the nucleic acid sequences described herein can be used to prepare two PCR primers from the 5′ and 3′ ends of the individual's DNA and subsequently sequence it. Panels of corresponding DNA sequences from individuals, prepared in this manner, can provide unique individual identifications, as each individual will have a unique set of such DNA sequences due to allelic variation. The sequences of the present invention can be used to obtain such identification sequences from individuals and from tissue. The nucleic acid sequences of the invention uniquely represent portions of the human genome. Allelic variation occurs to some degree in the coding regions of these sequences, and to a greater degree in the non-coding regions. It is estimated that allelic variation between individual humans occurs with a frequency of about once per 500 bases. Thus, each of the sequences described herein may be, to some degree, used as a standard against which DNA from an individual can be compared for identification purposes.

c. Forensic Biology

In addition the sequences described herein can be used in forensic biology. Forensic biology is a scientific field employing genetic typing of biological evidence found at a crime scene as a means for positively identifying, for example a perpetrator of a crime. To make such an identification, PCR-based technology can be used to amplify DNA sequences taken from very small biological samples such as tissues, (e.g. hair, skin, or body fluids). The amplified sequence can then be compared to a standard thereby allowing identification of the origin of the biological sample.

The sequences of the present invention can be used to provide polynucleotide reagents (e.g. PCR primers) targeted to specific loci in the human genome, which can enhance the reliability of DNA-based forensic identifications by, for example, providing another “identification marker” (i.e. another DNA sequence that is unique to a particular individual.) The nucleic acid sequences described herein can further be used to provide polynucleotide reagents e.g. labeled or labelable probes, which can be used in, for example, an in situ hybridization technique, to identify a specific tissue. This technique can be exceedingly useful in cases where a forensic pathologist is presented with a tissue of unknown origin. Panels of such probes can be used to identify tissue by species and/or organ type.

S. Predictive Medicine

Portions or fragments of the polynucleotide sequences of the invention can be used for predictive purposes to thereby treat an individual prophylactically.

1. Diagnostic /Prognostic Assays

One method of detecting the presence or absence of a polypeptide or nucleic acid in a biological sample is to expose that sample to an agent that recognizes the entity in question. A preferred agent for detecting niRNA or genomic DNA is a labeled nucleic acid probe capable of hybridizing to the sequence one is attempting to detect (for instance, the sequence of the invention). The nucleic acid probe can be, for example, a full length cDNA, or a portion thereof such as an oligonucleotide of at least 15, 30, 50, 100, 250, or 500 nucleotides in length and sufficient to specifically hybridize under stringent conditions to a mRNA or genomic DNA encoding the invention. The term “labeled” in this context refers to modifications in said sequences including, but not limited to, biotin labeling that can then be detected with a fluorescently labeled streptavidin, or ³²P labeling.

A preferred agent for detecting a polypeptide of the invention is an antibody or peptide capable of binding to the invention, preferably an antibody with a detectable label. Antibodies can be polyclonal or more preferably, monoclonal. An intact antibody, or a fragment thereof (e.g. a Fab or F(ab)₂) can be used. The term “labeled” in this context refers to direct labeling of the probe or antibody by coupling (i.e. physical linking) a detectable substance to the probe or antibody, such as a fluorescent labeled moiety or biotin.

The detection methods of the invention can be used to detect niRNA, protein, or genomic DNA in a biological sample in vitro as well as in vivo. For example, in vitro techniques for detection of mRNA include (but are not limited to) Northern Blot hybridization and in situ hybridizations. In vitro techniques for detection of a polypeptide of the invention include enzyme linked immunosorbent assays (ELISA's), Western blots, immunoprecipitations, and immunofluorescence.

The invention also encompasses kits for detecting the presence of a polypeptide or nucleic acid of the invention in a biological sample. Such kits can be used to determine if a subject is suffering from or is at increased risk of developing a disorder associate with aberrant expression of a polypeptide or polynucleotide of the invention. For instance, the kit can comprise a labeled compound or agent (as well as all the necessary supplementary agents needed for signal detection e.g. buffers, substrates, etc.) capable of detecting the polypeptide, or mRNA in the sample (e.g. an antibody which binds the polypeptide or a oligonucleotide probe that binds to DNA or mRNA encoding the polypeptide).

The methods of the invention can also be used to detect genetic lesions or mutations in a gene of the invention, thereby determining if a subject with the lesioned gene is at risk for a disorder characterized by aberrant expression or activity of an agent of the invention. In preferred embodiments, the methods include detecting the presence or absence of a genetic lesion or mutation characterized by at least one alteration affecting the integrity of the agent of the invention. For example, such genetic lesions or mutations can be detected by ascertaining the existence of at least one of: 1) a deletion of one or more nucleotides from a gene; 2) an addition of one or more nucleotides to a gene; 3) a substitution of one or more nucleotides of the gene; 4) a chromosomal rearrangement of the gene; 5) an alteration in the level of a messenger RNA transcript of the gene; 6) an aberrant modification of the gene, such as of the methylation pattern of the genomic DNA; 7) the presence of a non-wild type splicing pattern of a messenger RNA; 8) a non-wild type level of the protein encoded by the gene; 9) an allelic loss of the gene; and 10) an inappropriate post translational modification of the protein encoded by the gene. Many techniques can be used to detect lesions such as those described above. For instance, mutations in a selected gene from a sample can be identified by alterations in restriction enzyme cleavage patterns. In this procedure, sample and control DNA is isolated, digested with one or more restriction endonucleases, and fragment length sizes (determined by gel electrophoresis) are compared. Observable differences in fragment length sizes between sample and control DNA indicates mutations in the sample DNA. Additional techniques that can be applied to detecting mutations include, but are not limited to, detection based on direct sequencing, PCR-based detection of deletions, inversions, or translocations, detection based on mismatch cleavage reactions (Myers, R. M. et al. (1985) “Detection of single base substitutions by ribonuclease cleavage at mismatches in RNA:DNA duplexes.” Science 230:1242), and detection based on altered electrophoretic mobility (e.g. SSCP, see, for example, Orita, M. et al. (1989) “Detection of polymorphisms of human DNA by gel electrophoresis as single-strand conformation polymorphisms.” PNAS 86:2766).

2. Pharmacogenetics

Pharmacogenetics deals with clinically significant hereditary variation in the response to drugs due to altered drug disposition and altered action in affected persons (see Linder, M. W. et al. (1997) “Phannacogenetics: a laboratory tool for optimizing therapeutic efficiency.” Clin Chem. 43(2):254-266). In general, two types of pharmacogenetic conditions can be differentiated. There are genetic conditions transmitted as a single factor altering the way drugs act on the body, referred to as “altered drug action”. Alternatively, there are genetic conditions transmitted as single factors altering the way the body acts on drugs (referred to as “altered drug metabolism”). These two conditions can occur either as rare defects, or as polymorphisms. For example, glucose-6-phosphate dehydrogenase deficiency is a common inherited enyzmopathy in which the main clinical complication is haemolysis after ingestion of oxidant drugs (e.g. anti-malarials, sulfonamides etc.).

The activity of drug metabolizing enzymes is a major determinant of both the intensity and duration of drug action. The discovery of genetic polymorphisms of drug metabolizing enzymes (e.g. N-acetyltransferase 2 (NAT2) and cytochrome P450 enzymes (CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM which all lead to the absence of functional CYP2D6. Poor metabolizers of this sort quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. At the other extreme are the socalled ultra rapid metabolizer who do not respond to standard doses. Recently, the molecular basis of ultra rapid metabolism has been identified to be due to CYP2D6 gene amplification.

Thus the in the context of pharmacogenetics, an agent of the invention can be used to determine or select appropriate agents for therapeutic prophylactic treatment of the individual. In addition, pharmacogenetic studies can be used to apply genotyping of polymorphic alleles encoding drug-metabolizing enzymes to the identification of an individuals drug responsiveness phenotype.

3. Monitoring of Effects During Clinical Trials

Monitoring the influence of agents that effect the expression or activity of a polypeptide or polynucleotide of the invention can be applied in clinical trials. For example, the effectiveness of a drug directed toward a target identified by the invention and intended to treat a particular ailment, can be monitored in clinical trials of subjects exhibiting said ailment by monitoring the level of gene expression of the target, activity of the target, or levels of the target of the invention. Thus in a preferred embodiment, the present invention provides a method for monitoring the effectiveness of treatment of a subject with an agent by comprising the steps of (i) obtaining a pre-administration sample from a subject prior to administration of the agent; (ii) detecting the level of the polypeptide or polynucleotide of the invention in the pre-administration sample; (iii) obtaining one or more post-administration samples from the subject; (iv) detecting the level or activity of said target of the invention in the post-administration samples, (v) comparing the level of said target of the invention in the post administration sample with levels in the pre-administration samples, and (vi) altering the administration of the agent to the subject accordingly.

### EXAMPLES

The following examples are intended to further illustrate certain preferred embodiments of the invention, and are not limiting in nature

### Example 1

### HeLa and RV-14 Viral Cultures

HeLa cells (human cervical adenocarcinoma cells, ATCC CRL-1958) were propagated as monolayers in DMEM media (Gibco BRL) supplemented with 10% FBS, L-Glutamine (2 mM final), non-essential amino acids (1×), and Sodium Pyruvate (1 mM). In some cases, Pen/Strep (1×, 100 ug/ml ea.) was added to the cultures prior to retroviral transductions and/or RV-14 infections to minimize the risk of bacterial contamination. Cultures were grown at 33, 37, or 39° C. (5% CO₂) in standard tissue culture flasks.

Human Rhinovirus-14 (RV-14) was obtained from the American Tissue Culture Center (ATCC, #VR 284). To obtain stocks of rhinoviral supernatants for perturbagen screens, sub-confluent plates of HeLa cells growing at 33° C. were infected with RV-14 in the presence of 2% serum and allowed to propagate until >95% cell death was observed (˜3-7 days). Subsequently, the cells and the media were collected, freeze/thawed two times at −80° C. and centrifuged at 1200×g to remove cellular debris. This viral stock was stored at −80° C. in 5 ml aliquots. Virus thawed for use was kept at 4° C. for up to one month. Titering viral supernatants was accomplished by determining the TCID₅₀ (Tissue culture infectious dose necessary for 50% of cultures to be infected, see, for example Reed and Muench, Am. J. Hyg., vol. 27, pages 493-497 (1938), or U.S. Pat. No. 6,127,422)). Specifically, serial 10-fold dilutions of RV-14 viral supernatants were added to rows of a 96 well microtiter plate which had been seeded the day before with 2000 HeLa cells per well. The virus and HeLa cells were incubated for seven days, upon which time individual wells were scored for infection either by microscopic examination or by fixation with methanol and staining with crystal violet.

### Example 2

### Neutralizing RV-14 with mAb

Monoclonal antibodies directed against critical epitopes have been shown to be effective in neutralizing rhinoviral infection (see, for example, Sherry, B. et al. (1986) “Use of monoclonal antibodies to identify four neutralization immunogens on a common cold picornavirus, human rhinovirus 14”. J Virol. 57(1):246-57. Smith, T. J. et al. (1996) “Neutralizing antibody to human rhinovirus 14 penetrates the receptor-binding canyon.” Nature 383(6598):350-4.). The hybridoma cell line producing “mAb17” (a gift of T. Smith, Purdue University, West Lafayette Indiana) was used to generate large amounts of an RV-14 neutralizing monoclonal antibody. Cells were grown in an Integra Biosciences Cell line CL-350 Passive Membrane Bioreactor according to the manufacture's instructions in DMEM media containing 10% Fetal Calf Serum, 20 mM Hepes and 45 nM beta-mercaptoethanol. Every 3 to 4 days, 5 mls of the media containing cells, cellular debris, and the neutralizing antibody were collected and spun to remove insoluable material. Since it proved unnecessary to purify the antibody, this material was pooled, titered for neutralizing activity as described below, and frozen at 80° C. in aliquots.

The RV-14 neutralizing antibody is used during each round of selection to prevent super-infection by progeny virus produced from the initial innoculum of virus. Since it is not toxic to uninfected cells even at the highest concentrations tested, it was only necessary to have an excess of Ab to virus produced during an infection cycle. An empirical assay for determining a neutralizing titer was developed using a crude visual readout of virus cytotoxicity in a 96 well microplate. A Rhinovirus Inhibitory Unit (RIU) was defined as the amount of Ab needed to completely inhibit cytotoxicity to 1×10⁴H1-Hela cells caused by 2×10⁵ TCID₅₀ virus (from a single reference stock of titer 6.3×10⁶ TCID₅₀/ml) at 24 hrs of infection. After accurately determining the inhibitory titer of one particular reference stock of mAb other samples were titered by comparison to this material. Alternative methods for measuring neutralizing titer are known in the art (see, Sherry B, (1986) and Sherry, B. and Rueckert, R. R. (1985) “Evidence for a least two dominant neutralization Antigens on Human Rhinovirus 14” J. Virol. 53(1):137-143). To confirm that the amount of antibody added to a large scale selection is sufficient these same methods can also be used to determine the titer of the non-neutralized fraction of virus produced in the course of the infection.

### Example 3

### Preparation, Packagying and Titer of a cDNA Library

Using techniques that are familiar to individuals in the art, randomly primed cDNA libraries were used as a source of sequences encoding putative anti-Rhinovirus antiviral agents. As one non-limiting example of how to construct such a library, polyA mRNA derived from placental tissue was PCR amplified using a random 9-mer linked to a unique SfiI sequence (“SfiA”), followed by an additional sequence that is used later for library amplification (OVT 906: 5′ ACTCTGGACTAGGCAGGTTCAGTGGCCAT TATGGCC(N)₉). The product of this reaction was size selected (>400 base pairs) and subjected to RNAse A/H treatment to remove the original RNA template. The remaining single stranded DNA was then subjected to a second round of PCR using a random hexamer nucleotide sequence linked to a second unique SfiI sequence (“SfiB”) which was again followed by an additional sequence for future library amplification: (OVT 908: 5′ AAGCAGTGGTGTCAACG CAGTGAGGCCGAGGCGGCC (N)₆). The final product of this reaction, a double stranded cDNA, was blunted/filled with Klenow Fragment (New England BioLabs), size selected, PCR amplified (OVT 909: 5′ ACTCTGGACTAGGCAGGTTCAGT and OVT 910:5′ AAGCAGTGGTGTCAA CGCAGTGA), digested with SfiI (New England BioLabs), and inserted into a retroviral vector (pVT 352.1, pBabe). As a result of these procedures, the sequences encoding the perturbagens were inserted at the 3′ end of the non-fluorescent variant of EGFP (dead GFP or “dEGFP”). Expression of the dEGFP-perturbagen fusion gene (as well as the neomycin resistance gene present in the retroviral vector) was driven by the 5′ LTR of pBabe. The library (˜12×10⁶ in size) was then packaged in 293 gp cells (laboratory of I. Verma) and retroviral supernatant was collected over the course of the following 48-72 hours. Two methods are commonly used for retroviral packaging. In the first technique, the retroviral library is co-transfected with VSV-G envelope expression plasmid into 293 gp packaging cells (gift of I. Verma, Salk Institute) using LIPOFECTAMINE (ife Technologies). In this technique, 3×10⁶ cells of the packaging cell line (293 gp) are seeded into a T175 flask. On the next day, two tubes are prepared, one containing 15 □g of library DNA and 10 □g of envelope plasmid (pCMV-VSV.G-bpa) in 1.5 ml DMEM (serum free), the second containing 100 □g of LIPOFECTAMINE in 1.5 ml DMEM (serum free). These tubes are incubated at room temperature for 30 minutes, mixed and incubated for another 30 minutes. Subsequently the mix is added to 17 mls of serum free DMEM. This mix was added to previously plated 293 gp cells which had been washed with serum free media. Following a 4 hour incubation at 37° C. The transfection mix was removed and the cells are washed once in DMEM containing 10% serum and left in the same media. After 72 hours at 37° C. the media (now referred to as “viral supernatant”) is collected, filtered through a 0.45 μm filter and frozen at −80° C. It is possible to make a second collection of virus which has a comparable titer by adding 15 mls of DMEM (10% serum) back to the cells and incubating a further 24 hours.

As an alternative methodology, retroviral DNA can be packaged using a technique that is referred to herein as the “CaCl₂ Method”. In this method, 5×10⁶ cells of the packaging cell line (293 gp) are seeded into a 15 cm² flask on Day 1. On the following day, the media is replaced with 22.5 mls of modified DMEM. Subsequently, a single tube carrying 22.5 μg of retroviral library DNA and 22.5 μg of envelope expression plasmid (pCMV-VSV.G-bpa) is brought to 400 μl with dH₂O, to which is added 100 μof CaCl₂ (2.5M) and 500 μl of BBS (drop-wise addition, 2×solution=50 mM, BES (N,N-bis(2-hydroxyethyl)-2-aminoethane-sulfonic acid), 280 mM NaCl, 1.5 mM Na₂HPO₄, pH 6.95). After allowing this retroviral mixture to sit at room temperature for 5-10 minutes, i.e. is added to the 293 gp cells in a drop-wise fashion, and the cells are then incubated at 37° C. (3% CO₂) for 16-24 hours. The media is then replaced and the cells are allowed to incubate for an additional 48-72 hours at 37° C. At that time, the media containing the viral particles is then collected, filtered through a 0.45 μm filter and frozen down at −80° C. Retroviral supernatant can subsequently be thawed and used directly to infect HeLa cells.

Transduction of the cDNA library or sublibraries derived from the different rounds of selection followed standard procedures common to the art. In brief, 2×10⁶ H1-HeLa cells (grown at either 33 or 37° C.) were mixed with the pVT352.1 viral supernatant and plated in a T175 flask at 33° C. In different experiments the ratio of the volume of viral supernatant to total tissue culture media (20 mls DMEM containing 10% serum per T175) varied between 20%-50% vol/vol. To improve transduction, the viral supernatants were supplemented with polybrene at a concentration of 6 ug/ml. After a twenty-four hour incubation, the cells were washed and cultured with fresh media for two more days to allow expression of the genes carried on the transduced retroviral construct.

It is often useful to know what fraction of the cells was transduced with a retroviral vector. Although this can be determined by selecting for the antibiotic resistance marker carried by the vector, a more rapid, method for determining the percentage of transduced is to analyze the cells by flow cytometrey after staining with an antibody to the scaffold carrying the cDNA (in this case dead GFP). This method has the additional advantage of being internally controlled and highly quantitative. Specifically, a sample of 2×10⁶ cells were centrifuged for 5 minutes at 400×g and then fixed with 1% formaldehyde in PBS (1 ml, 20 min, room temperature). The cells were then re-pelleted, treated with ice-cold methanol (1 ml, 10 min), washed once with PBS, and then resuspended in 500 □l of 10% goat serum in PBS for thirty minutes to block nonspecific antibody binding sites. Following the blocking procedure, samples were incubated for 30 minutes with 100 □l primary antibody (a mixture of two mouse anti-GFP monoclonal antibodies, Boehringer-Mannheim) in 10% goat serum/PBS. Each sample was then washed once in 10% goat serum/PBS, resuspended in 100 ul of the 20 antibody (goat anti-mouse labeled with FITC, Pharmingen) and incubated an additional 30 minutes in the dark to prevent photo-bleaching of the FITC chromophore. Samples were then washed once in 10% goat serum in PBS and scanned on a Coulter EPICS XL analyzer to determine the percentage of cells expressing dead GFP. Over the course of these experiments, the percentage of infected cells varied between 85 to 99% . In some experiments the amount of virus supernatent was titered down (to 1%) because the calculation of MOI is more accurate when the fraction of infected cells is small (1% to 10%). From this data the calculated MOI of most large scale transductions fell between 1.6 to 3.5 virus transducing particles per cell.

### Example 4

### Isolation of Perturbagens that Block the Rhinoviral Lifecycle

To isolate perturbagens that inhibited rhinovirus growth, H1-HeLa cells were transduced with cDNA libraries or subpools thereof, infected with RV-14, and screened for viral resistant cells. Cells that survived the RV-14 infection were used as a source of DNA from which to PCR the cDNA inserts. The product of each PCR reaction was then used to create a new sublibrary. During the cycles of enrichment the total number of flasks used, and number of cDNA clones transduced. The conditions in each flask were largely constant.

H1-Hela cells were plated in T175 flasks and simultaneously transduced with the retroviral supernatant containing the cDNA library (this retroviral infection step is referred to herein as a transduction to distinguish it from the subsequent infection with rhinovirus). After three days of growth to allow expression of the dead GFP-cDNA fusion the cell number increased 6 fold. Subsequently, the cells were trypsinized and counted. An aliquot of 5×10⁶ cells was then plated in each T175 and infected with a sufficient quantity of RV-14 to kill 99 to 99.9% of the population.

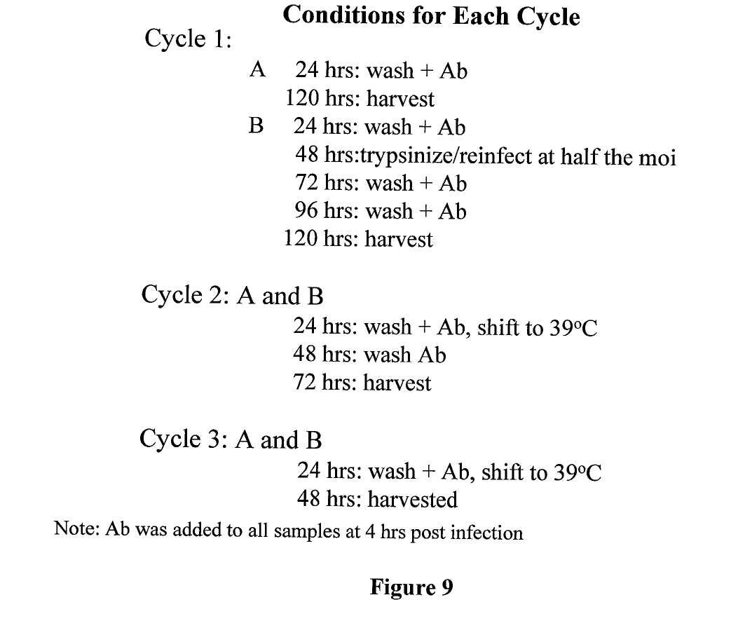

The cells infected in Cycle 1 screen were split into two groups. Half of the cells (referred to herein as Group A) were allowed to incubate for a period of five days before being harvested. Group B cells were washed at t=24 hrs, re-infected a second time at t=48 hrs, and then harvested 72 hours later (total incubation time=5 days). Both Group A and Group B populations were treated in a similar fashion in successive rounds of cycling (see FIG. 9).

To obtain a consistently high infection and killing rate (i.e. >99%) over the course of these experiments, medium-scale (1 T175 flask per sample) test infections were performed with each new viral stock. The amount of virus added was titrated around the calculated MOI of 10 to determine the minimum amount necessary to ensure that at least 99% of the cells would be infected/killed in large-scale perturbagen screens. In addition, several procedures were used to ensure that cells were not subjected to an uncontrolled secondary infection with a potentially high MOI. For instance, four hours post infection, a neutralizing monoclonal antibody was added to the media to inactivate virus released from cells. The amount of antibody added was estimated based on calculations that assumed a burst size of 40 virus per cell. Twenty-four hours after RV-14 infection, the media containing the original inoculate of infectious viral particles was removed, the flasks were washed with sterile PBS to remove the floating and loosely adherent dead cells, and fresh media containing additional antibody (one tenth of the original quantity) was added to the culture. In addition, the cultures were shifted to 39° C. at the twenty-four hour time point. Previous studies have shown that elevated temperatures are not harmful to the HeLa cell cycle, yet suppress infection of the cells by RV (see, for example, Conti, C. et al. (1999) “Antiviral Effect of Hyperthermic Treatment in Rhinovirus Infection.” Antimicrobial Agents and Chemotherapy 43(4):822-829.). The high temperature block to rhinovirus replication is not precisely mapped, however it does block RV-14 mediated cytotoxicity if it is imposed before 6 hours post infection.

Following each cycle, live, adherent cells were collected and used to prepare a new sublibrary. The procedure of retrieving the library sequences after each successive round of selection minimizes the background levels of viral-resistant cells that can accumulate due to mutations in the host chromosomal DNA. As one example of generating a perturbagen sublibrary, adherent cells that had been harvested by trypsinization of the culture flask were collected by centrifugation and used to prepare genomic DNA (Trizol, Reagent, Life Technologies). The library DNA was then recovered by two stages of PCR amplification using oligonucleotides that contained homology with sequences flanking the cDNA insertion site (oVT181: 5′ GGATCACTCTCGGCATGGACGAG and oVT178: 5′ ATTTTATCGATGTTA GCTTGGCCATT). Specifically genomic DNA from 10,000 to 700,000 cells was added to a 100 ul PCR containing 2.5 mM MgSO₄, 10 μM primers, 0.2 mM dNTPs, 100 ug/ml BSA and 10 units HiFi Taq polymerase (Life Technologies) in 1×buffer supplied by the manufacturer. This was denatured at 94° C. for 5 minutes and then amplified by 20 cycles of: 94° C. 15 seconds, 68 C. for 2:20 minutes followed by 5′ at 68 C. Ten microliters of this reaction was further amplified in a 200 ul PCR reaction under the same conditions for a number of cycles determined by cycle course titration (generally 16 cycles).

The PCR product was then purified by phenol/chloroform extraction and ethanol precipitation. Subsequently, each sample was digested with SfiI (New England Biologicals), purified through a Chroma Spin 200 column (Clontech) and directionally ligated (T4 ligase, Boehinger Mannheim) into the original vector (pVT352.1) that had been cut with Sfi and purified by agarose gel electrophoresis. Subsequently, this material was transformed into bacteria by electroporation (DH10B, Electromax, Gibco) and plated on LB-Amp plates for selection of colonies that contain a member of the sublibrary. Ampicillin resistant colonies (Amp^{R}) were then pooled and plasmid DNA purified using a Maxiprep (Qiagen). This cDNA sublibrary was then re-packaged in 293 gp cells in preparation for subsequent rounds of cycling and enrichment in HeLa cells.

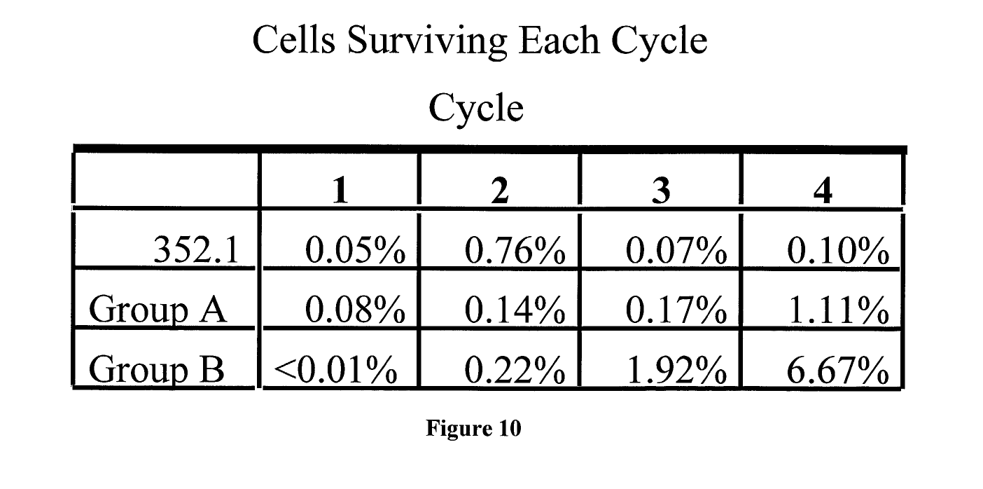

The fraction of HeLa cells surviving RV-14 infection changes dramatically over the course of four rounds of cycling/enrichment . Initially, the number of surviving cells observed in library containing populations mirrored the number observed in control studies (i.e. RV-14 infected cells w/o cDNA library) and numbered less than 0.1% (<1:1000). By the end of four rounds of cycling/enrichment the numbers of RV-14 resistant cells in the library-containing population increased 14-426 fold (depending upon the subpool being measured), and totaled between 1.1-6.6% of the total population at the end of Cycle 4 (FIG. 10).

Following each cycle of the selection, individual library clones were picked into microtiter plates for sequencing on an ABI sequencer. Clones that were observed at a high frequency were repackaged in 293 gp cells and tested individually in the biological assay for their ability to hinder RV-14 replication.

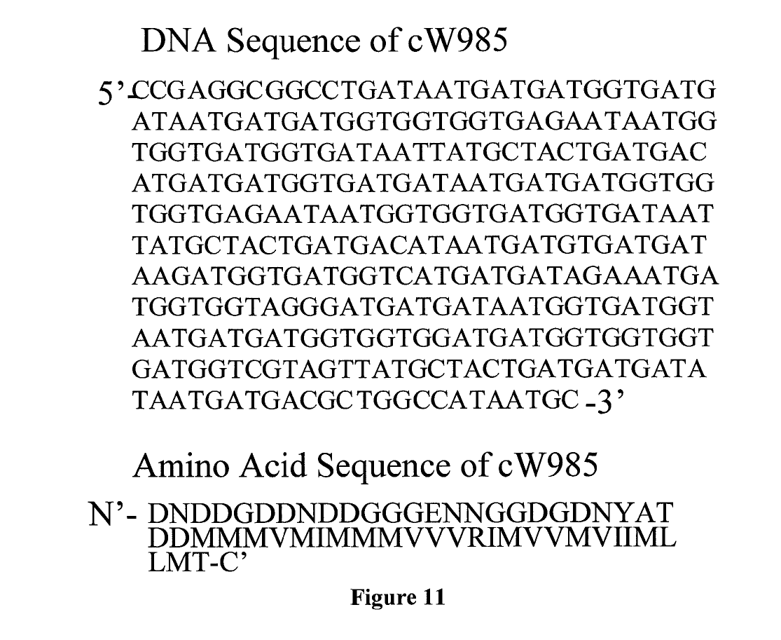

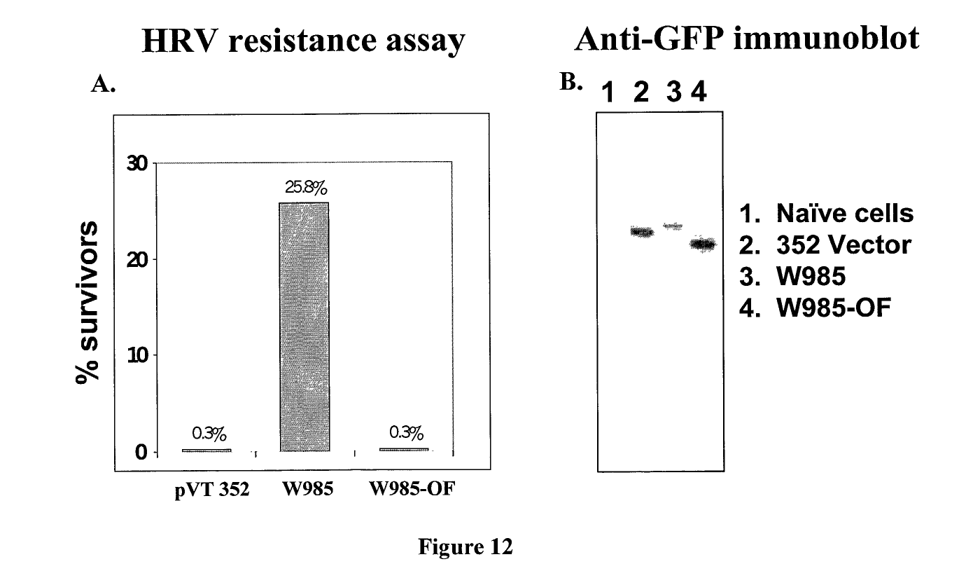

Several hundred clones isolated from cycles 4B, 3B, 2B, 3A, and 2A populations were sequenced to determine the representation of clones within the population. Based on clonal frequency and distribution data obtained from sequencing, twenty clones were chosen to be retested in the bio-assay for the ability to inhibit viral replication. One particular clone (represented herein by the example W985, FIG. 11) was found in multiple sort populations (F3A, F3B and F4B) and was represented 28 times out of the 623 clones sequenced. When W985 was reintroduced into HeLa cells and subsequently challenged with RV-14, a large fraction of the W985-containing population were observed to be resistant to viral-induced cell death. In different experiments, 20 to 60% of the W985 containing population was virus resistant compared to 0.1 to 0.8% virus resistance in the pVT352.1 vector control population. In contrast, HeLa cells transfected with an out-of-frame W985 sequence exhibited no anti-viral properties (FIG. 12), suggesting that the perturbagen acted as a peptide rather than as an RNA molecule.

### Example 5

### Modes of Action

Heat Shock Proteins

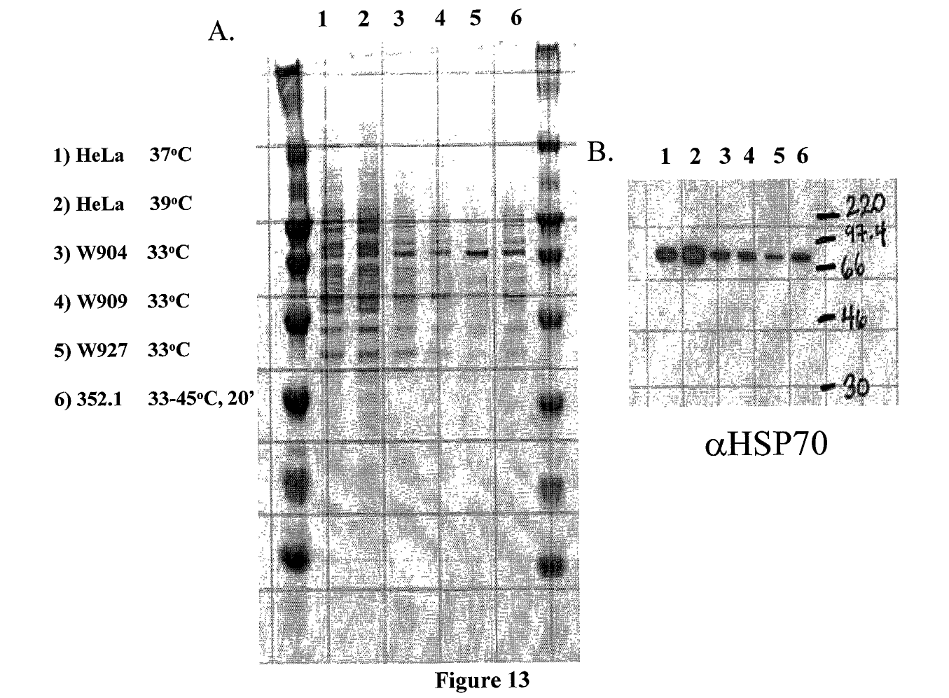

To determine whether the action of perturbagen W985 was mediated by increases in the intracellular concentrations of hsp70, cytosolic proteins purified from HeLa cells cultured under various conditions were probed on a Western Blot with antibodies that recognize hsp70. Specifically, HeLa cells were grown at 1) 37° C., 2) 39° C., 3) at 33° C. with W985 introduced under both moderate (3.5) and low (˜1.0) multiplicities of infection, and 4) at 33° C. with the control vector (pVT352.1). When soluble proteins isolated from each of these samples were examined for increases in hsp70 levels, only the control samples grown at 37° C. and 39° C. were observed to have an elevation in intracellular hsp70 concentrations (FIG. 13). HeLa cells containing the W985 perturbagen exhibited no alteration of intracellular hsp70 suggesting the mode of action of this perturbagen is not mediated through induction of the heat shock response.

Single Step Growth Curves

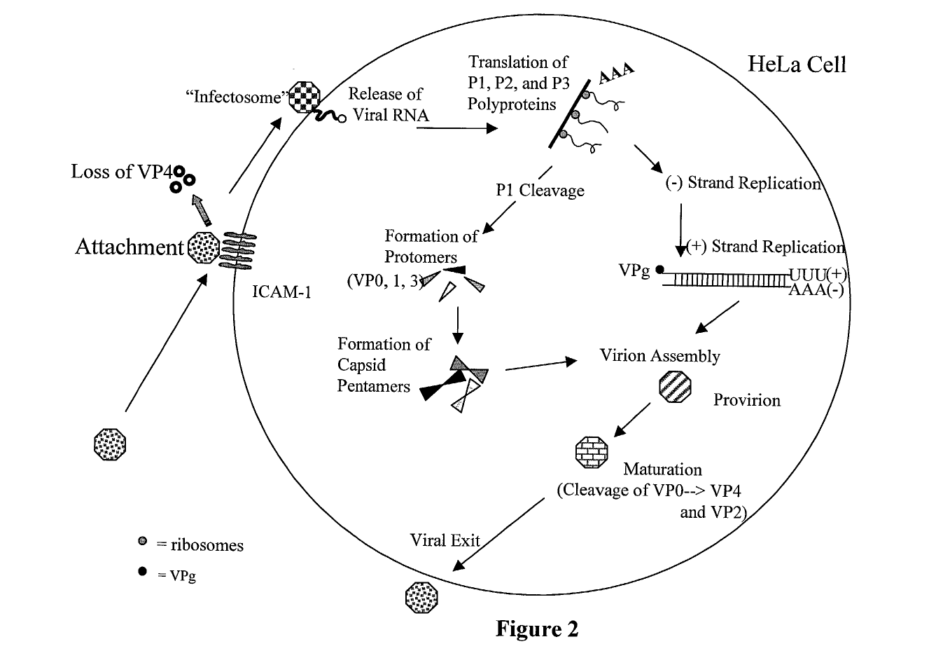

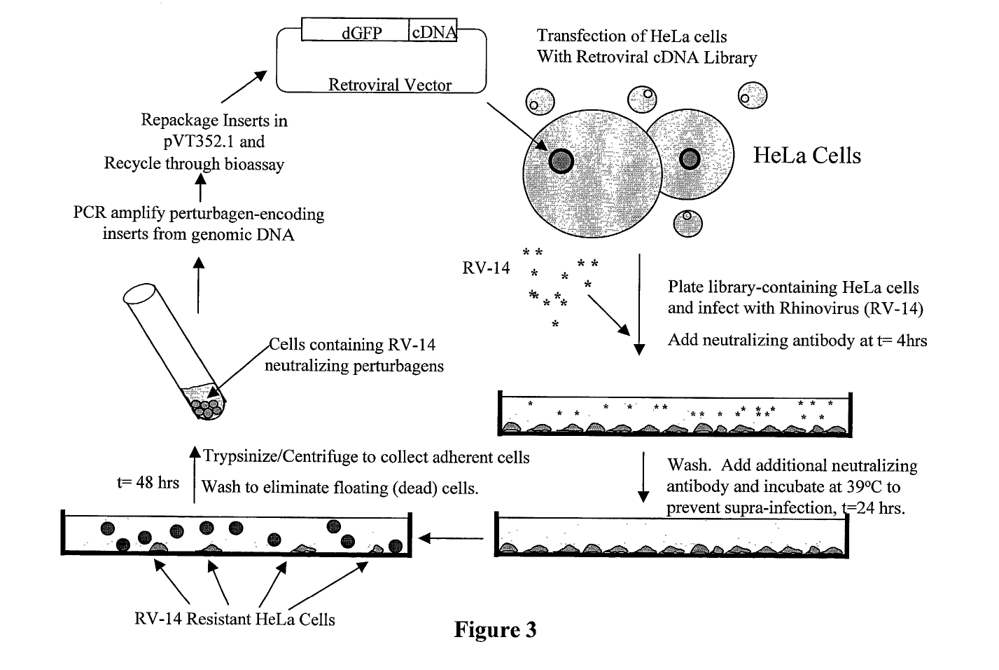

Replication of RV-14 requires a series of ordered steps (FIG. 2) many of which can be observed at the molecular level when the initial infection is synchronized. RV-14 completes its lifecycle in Hela cells (entry to appearance of progeny virus) in approximately 8 hours at 33° C. Since RV-14 is unable to kill W985-expressing cells during an infection the perturbagen most likely acts to block the virus at an early step in the life cycle. This block may be visible as a decrease in the burst size of the virus and/or a delay in the appearance of progeny virus in a synchronized infection. To assess this, single step growth curves were performed on H1-HeLa cells transduced with either the control vector or W985. To accomplish this, cells were trypsinized and resuspended in DMEM+10% FCS. Cells and virus were then mixed at a cell concentration of 0.5×10⁶ cells/0.5 ml and an MOI of 10 and incubated at 33° C. for 30 minutes. The cells were then washed twice (DMEM+2% FBS), centrifuged (400×g, 5 minutes) and then divided into 3 ml aliquots (0.5×10⁶ cells) which were incubated in T25 flasks at 33° C. (defined as t=0). Subsequently, at successive two-hour time points, individual flasks of cells were removed from the incubator and frozen at −80° C. Upon obtaining multiple samples in this fashion, the cells were thawed, centrifuged to clear the lysate, and analyzed to determine the TCID₅₀ of each time. Aliquots of the cell and virus mixture at the beginning of the incubation (t=30′) and just before the wash step (t=5′) were also titered.

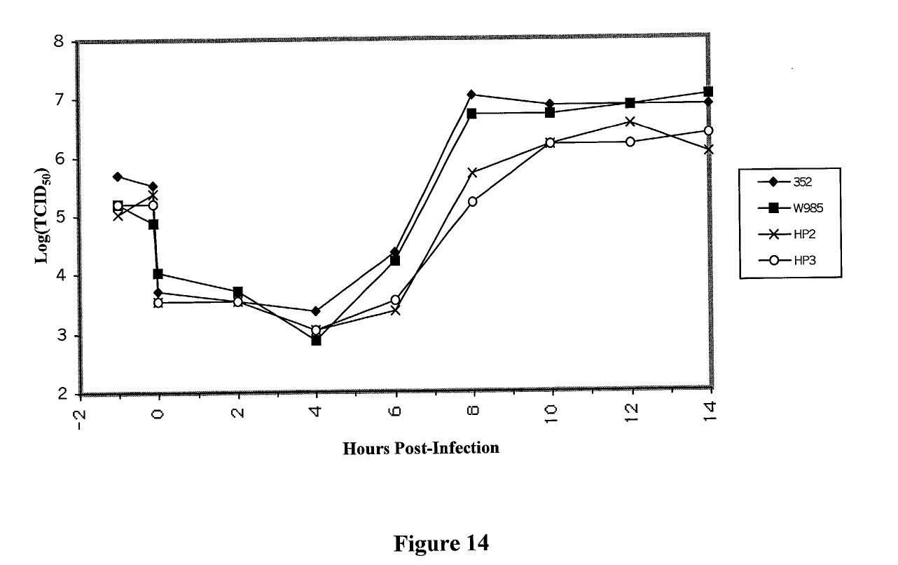

Results of the single step growth curves show that when a population of W985-containing H1-HeLa cells were infected with virus, the burst size was identical to that of cells transduced with the control vector at all times after infection (see FIG. 14). One possible mechanism that could reconcile this with the previous observation of W985 mediated cellular resistance to RV-14 is that heterogeneity exists within the W985 transduced population (e.g. due to retroviral insertion site). If, for example, one half of the cells survive, then the remaining half may go on to produce a normal burst of virus. A reduction of two to three fold in titer would be less than the measurement error of the TCID₅₀ assay.

To test whether heritable differences in virus resistance and/or virus production existed within the W985 containing H1-HeLa population, individual clones were isolated and retested for changes in the RV-14 burst size. To isolate such cell clones, single W985 transduced and neoR selected H1-Hela cells were deposited into the wells of a 96 well microtiter plate using the autoclone attachment of a Coulter Epics Elite cell sorter. After growth at 33° C. for 8 days the cells were trypsinized and replated in the same wells to disperse the colonies that had formed. Eight days later, the cells from 50 wells showing reasonable growth were transferred to two 24-well plates. An aliquot of each was infected for six hours and the approximate viral titer was determined by performing serial 10 fold dilutions of the virus in one row of a microplate. From these procedures, five lines that appeared to yield lower amounts of virus, were isolated and subsequently tested for burst size using the single step growth curve procedure described above. As shown in FIG. 14, analysis of two of the lines (W985 hp2, W985 hp3) showed that the yield of viral progeny at the 6 and 8 hour time points was approximately 50 fold lower than that of pools of cells transduced with either pVT352 or W985, suggesting that one or more stages of virus replication, was delayed in these lines. Interestingly enough, 10 hours after infection the level of virus in the HP clones approaches (but does not equal) that of the control cells, implying that W985 confers a delay rather than an absolute block on virus production.

Since the W985 high penetrance clones can be isolated at a frequency of at least 10%, it is unlikely that the observed RV-14 resistance embodied in these clones is the result of mutations in cellular genes which act either alone or in conjunction with the perturbagen to block RV-14 growth. A more likely scenario is that, factors such as retroviral insertion site in the host genome cause roughly ten percent of the population to express higher levels of the W985 peptide, and thus, are particularly resistant to RV-14 infection.

RNA Blot Analysis of Viral Synthesis

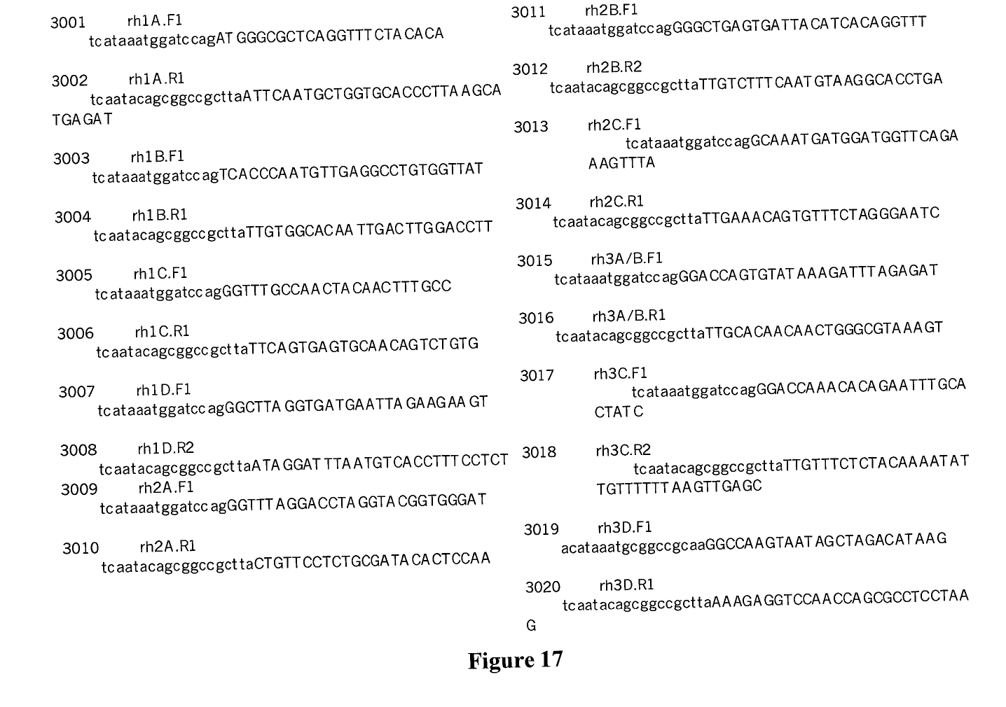

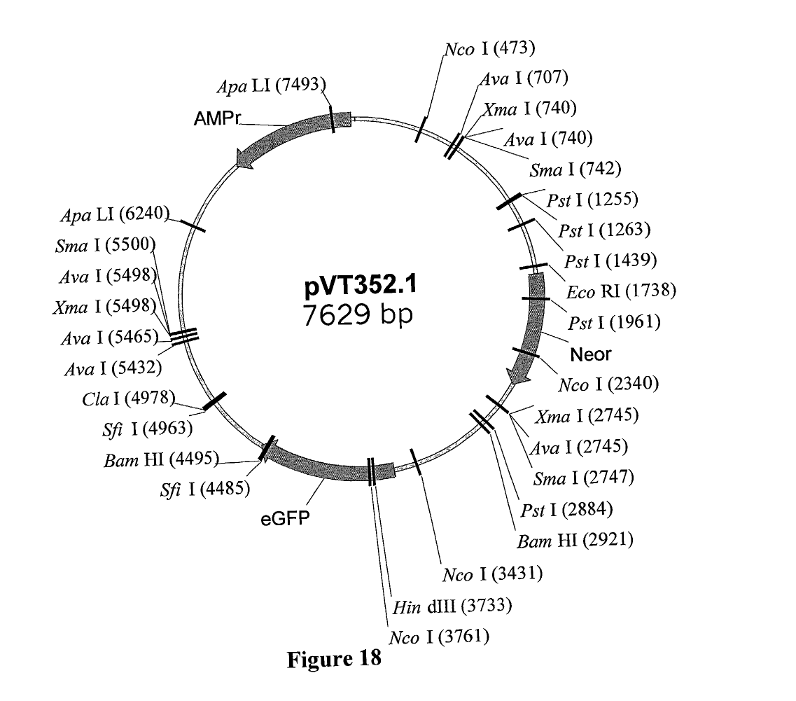

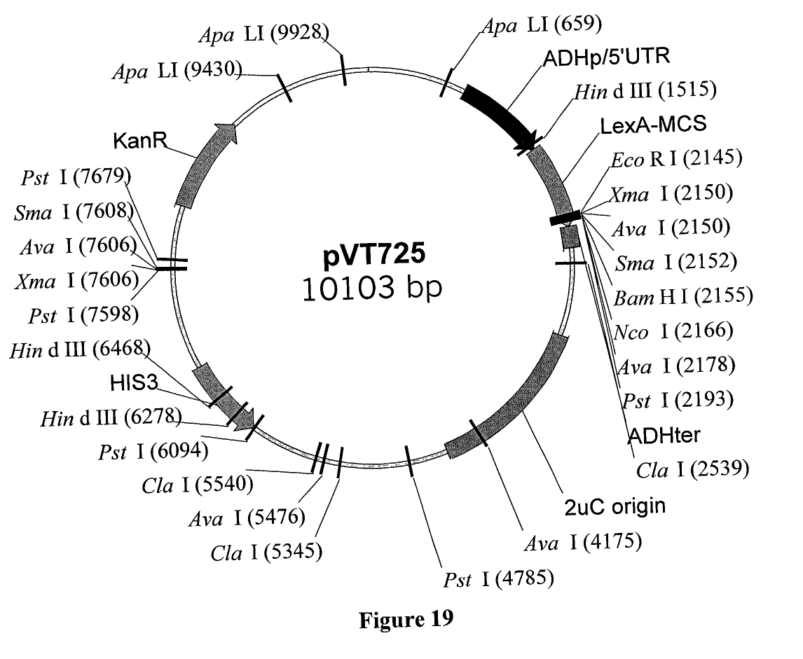

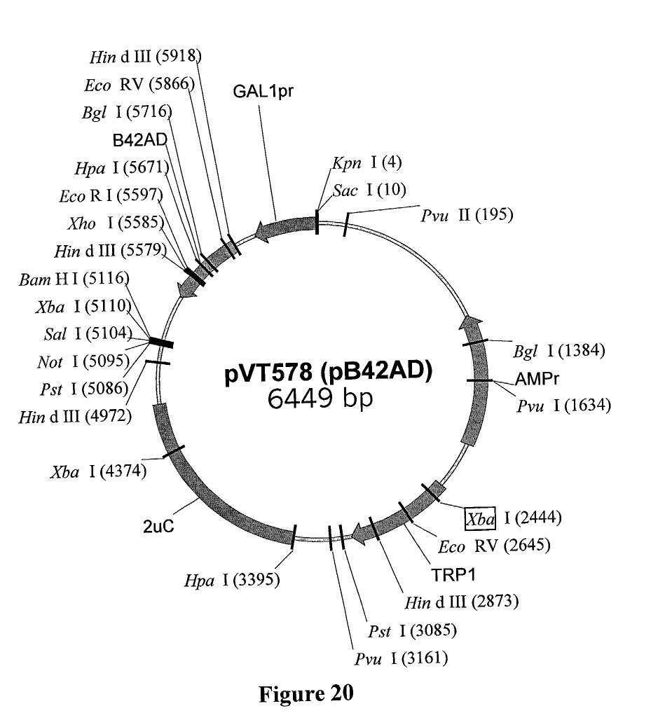

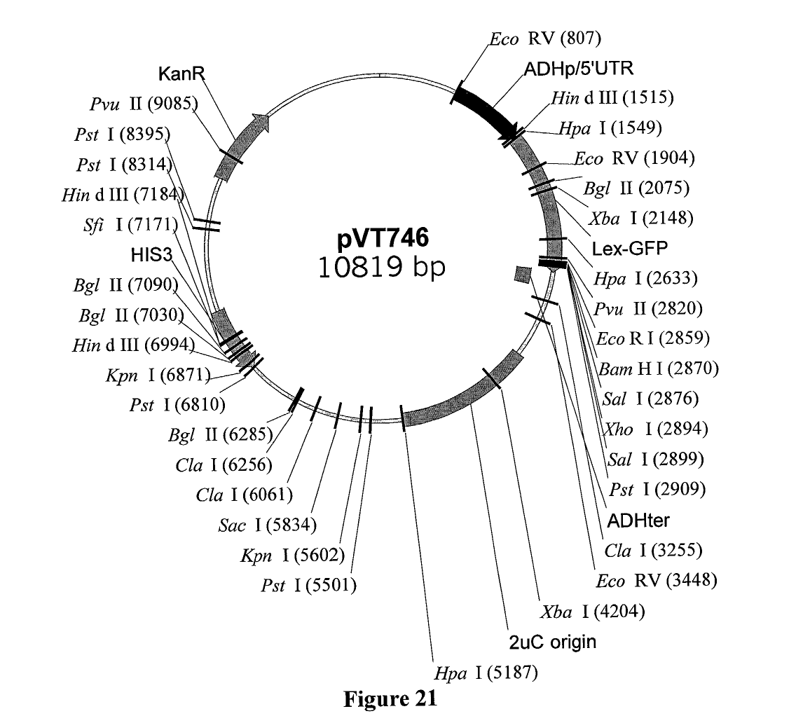

Because W985 hp3 cells show a kinetic delay in virus production these cells can be studied to determine the precise step in the virus life cycle which is blocked. As a first step, RNA blot analysis was used to determine the level of plus strand viral RNA. To accomplish this, an Rneasy Mini Kit (Qiagen) was used to prepare RNA from HeLa cells infected at defined times (1 million cells at MOI of 10 for 60′ in a 6 cm dish). The RNA was quantitated by OD₂₆₀ and 300K cell-equivalents were electrophoresed on a 1.0% formaldehyde/agarose gel. To judge the integrity of each RNA sample and equivalent loading of the 16S and 28S ribosomal RNAs in each lane, the gel was stained with EtBr and visualize under a UV light. The gel was then blotted onto Hybond XL membranes (Amersham-Pharmacia-Biotech), baked at 80° C. for 2 hr, and incubated for 1 hour in hybridization buffer (7% SDS, in 1 mM EDTA in 0.5M Na2PO4, prepared according to Church G M, Gilbert W. (1984) “Genomic sequencing” Proc Natl Acad Sci USA. 81(7):1991-5.). A single stranded radioactive DNA probe complementary to the plus (+) strand of the RV14 genome was then prepared (Bednarczuk T A, Wiggins R C, Konat G W (1991) “Generation of high efficiency, single-stranded DNA hybridization probes by PCR” Biotechniques. 10(4):478.) by performing 30 cycles of PCR in the presence of □³²P dCTP using pWR3.26 (which contains a cDNA copy of RV14 (a gift from Wei-Ming Lee, U W Madison), also see, Lee W. M. et al. (1993) “Role of maturation cleavage in infectivity of picomaviruses: activation of an infectosome”. J Virol. 67(4):2110-22) as a template and 6 primers (oVT numbers: 3004, 3008, 3014, 3016, 3018, 3020, see FIG. 17) complementary to the RV14 plus strand. Specifically, the PCR reaction contained 50 ng DNA, 0.2 □M of each primer, 1 unit Taq, 50 LM dATP, dGTP and dTTP, 50 □M ³²P □dCTP (3000 Ci/mmol, ICN), 2 mM MgSO₄, and 100 □g/ml BSA in 1×Hifi Taq buffer. The reaction was heated to 95° C. for 3′ followed by 30 cycles of 94° C. for 15 seconds, 50° C. for 20 seconds, and 72° C. for 2 minutes. Unincorporated nucleotides were then removed from the sample by centrifugation through a Micro-Bio spin column (Bio-Rad, Tris Buffer), and the blot was incubated with the probe for 16 hours (65° C.) in a total volume of 10 mls of hybridization buffer. After washing with 4 changes of 0.1×SSC at 65° C., the blot was exposed to a Molecular Dynamics phosphoimager screen and the resultant image quantitated using the manufacturers software.

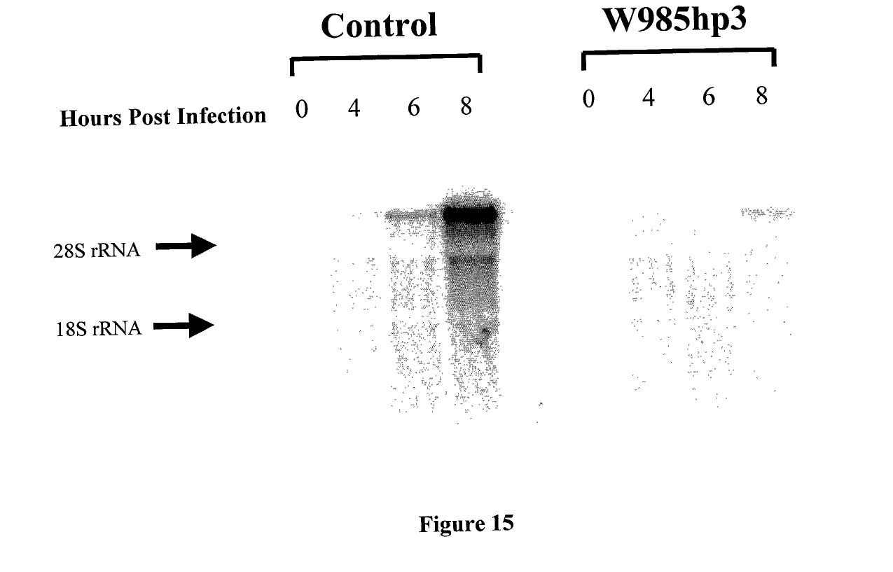

FIG. 15 shows that in a population of H1-HeLa cells infected with RV-14, the viral genome is detected by 4 hours and plateaus at 8 hours. In contrast, in the W985 hp3 cell clone expressing the anti-viral perturbagen, the levels of RV-14 are significantly repressed. This result is consistent with, and explains, the low virus yield at the same time points observed in the single step growth curve. The block imposed by the W985 perturbagen may still be several steps before this.

Plaque Assay

To determine whether the delay in viral maturation and observed reduction in viral burst size affected the ability of the RV-14 to spread to adjacent cells, a viral plaque assay was performed. To accomplish this, H1-Hela or W985 hp3 cells were plated in either a 10 cm or 6 cm dish (5×10⁶ cells or 1×10⁶ cells respectively) and cultured in DMEM +10% FCS at 33° C. The following day the media was removed and the cells were infected with variousdilutions of RV-14 in a total volume of either 5 ml or 2.5 ml (respectively) in DMEM +2% FCS. After 1 hour, the cells were overlayed with 8 mls (or 4 mls) of 1% molten agar in DMEM+2% FCS and then incubated at 33° C. for 3 days. To score the plates for viral plaques, the agar overlay was carefully removed, the cells were fixed with methanol, and then stained with 0.2% crystal violet in 10% phosphate buffered formalin.

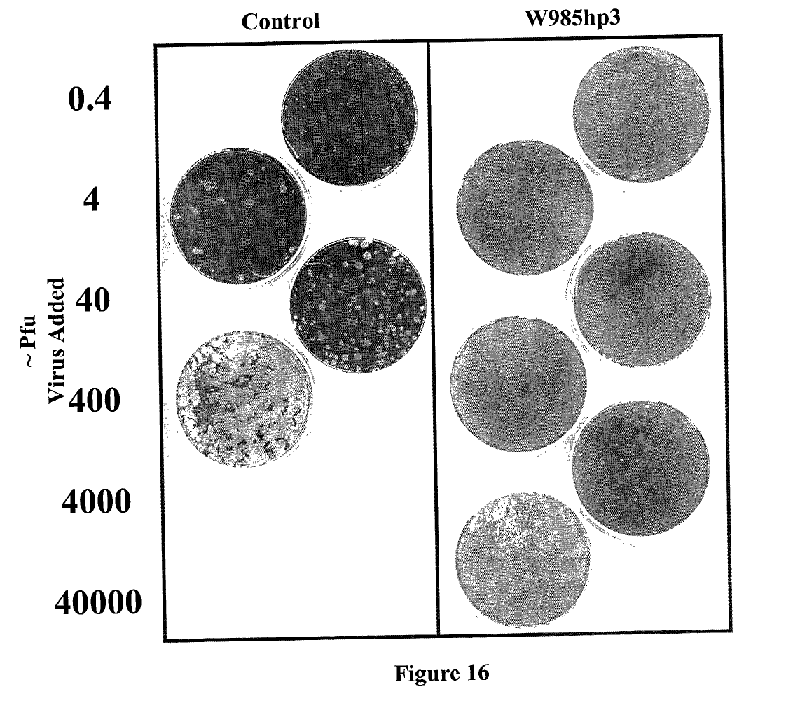

Results of the viral plaque assay revealed that, in contrast to the H1 HeLa cell control, plaque formation in the W985 hp3 cells was almost completely blocked in plates exposed to 0.4, 4, 40, and 400 Pfu (see FIG. 16). On the plate containing W985 hp3 exposed to 4,000 Pfu's, fewer than 40 small plaques were observed (in contrast control plates show nearly complete lysis at 400 Pfu). At the highest level of virus tested (40,000 Pfu) there was a general reduction in overall cell growth suggesting a nonspecific inhibition by high levels of virus.

This result is useful primarily because it opens the way to isolating mutant virus which are resistant to the action of the W985 hp3 perturbagen. Sequence analysis (and reconstruction of such a mutant can often provide valuable information about the mechanism by which the virus overcomes the inhibitory condition and therefore also about the inhibitory condition itself (Heinz, B. A. and Vance, L. M. (1995), Sherry, B. and Rueckert, R. R. (1985)).

### Example 6

### Target Identification

The following examples use perturbagen W985 to describe how the targets of viral-neutralizing perturbagens can be identified.

1. Two-Hybrid Methodology

Perturbagens that inhibit the viral lifecycle may be acting on either a viral or host cell target. For that reason, prospective perturbagens must be screened against both viral and host libraries to identify the perturbagen target.

2. Screening Viral Libraries

To identify the viral target of the W985 perturbagen, the polynucleotide sequence encoding W985 was cloned into the multiple cloning site of pVT578 (TRP⁺) using techniques common to the art. As a result of these procedures, the 53 amino acids of perturbagen W985 are fused in-frame with the C-terminus of the LexA activating domain which is, in turn, regulated by the Gal/Raf promoter. Concomitantly, a viral target library was constructed to identify any potential proteins that interacted with the W985 perturbagen. To accomplish this, ten of the polypeptides encoded by the RV-14 genome were RT-PCR amplified from the RV-14 RNA using viral-specific oligonucleotides flanked with the appropriate restriction sites (FIG. 17) and cloned into the MCS of pVT725 (HIS⁺). As a result of these procedures, each of the viral ORF's is fused in-frame with the Lex A binding domain which is, in turn, regulated by the ADH promoter.

Using conventional means the pVT578-W985 and pVT725-viral-library constructs are introduced into the appropriate yeast strain, for example, yVT 87 (Mat-α ura3-52, his3-200, trp1-901 , LexA_{op (xhd 6)}-LEU2-3,112), and selected on SD-His,

-Trp plates to identify transformants containing both plasmids. Viral proteins that interact with the W985 perturbagen are then identified by growing transformants on SD -His, -Trp, -Leu, plates containing galactose. Cells that are capable of forming colonies under these conditions are then collected and the associated viral ORF(s) are retested and sequenced using standard techniques.

3. Screening for Host Cell Target

To identify a host-cell target of the W985 perturbagen, the sequence encoding W985 was cloned into the pVT746 vector by gap-repair (Kobayashi I, (1992) “Mechanisms for gene conversion and homologous recombination: the double-strand break repair model and the successive half crossing-over model.” Adv Biophys 1992;28:81-133). As a result of these procedures, the W985 polypeptide is fused in-frame with the C-terminus of GFP that is, in turn, fused to the DNA binding domain of LexA. This construct, LexABD-GFP-W985, was then introduced into yVT 87 (Mat-α, ura3-52, his3-200, trp1-901 , LexA_{op (X6)}-LEU2-3, 112) and mated to yVT 99 (MAT-α, ura3, his3, trp1, leu2::lexAop(x6)-LEU2 lys2::lexAop(8× or 2×)-URA3) that contains a HeLa cDNA libraries (Life Technologies, Cat # 11287018) fused downstream of the GAL4 AD-protein. The mated mixture was first plated on SD-His-Trp plates to select and propagate diploids. Subsequently, diploids containing both constructs were then plated on SD-His, -Trp, -Leu, -Ura selection plates to select for cDNA's that bind to the W985 clone. Cells that formed colonies under these conditions were then collected and the associated target cDNA(s) was isolated by transforming the associated plasmid back into bacteria and growing said cells under conditions that selected for the presence of the cDNA (i.e. +Amp).

4. Immunoprecipitation

Viral targets of perturbagen W985 can also be identified by co-immunoprecipitation. Specifically, 10⁶ virally-infected cells containing the scaffolded (GFP-linked) perturbagen of interest are trypsinized, recovered by centrifugation, and washed in PBS containing 100 uM PMSF/1×Protease inhibitor cocktail. Subsequently the cells are lysed (4° C.) by resuspension in an immunoprecipitation buffer (IP buffer) containing 1% Triton X-100, 150 mM NaCl, 10 mM Tris HCl pH 7.4, 1 mM EDTA, 1 mM EGTA, 0.2 mM Na ortho-vanadate, 0.5% Na deoxycholate, 0.5% NP-40, 100 mM PMSF and 1×Protease Inhibitor cocktail. Following centrifugation (13K for 10 min at 4° C.), the lysate is then cleared by adding lug mouse IgG antibody (e.g. Mouse monoclonal IgG1ₖ Clone 7.1 and 13.1, Roche) plus 20 ul Protein A/G plus agarose (Santa Cruz Biologics) at 4° C., 1 hr. The sample is the centrifuged (2500 RPM for 5 min at 4° C.) and the supernatant is treated with lug of anti-GFP monoclonal antibody (Roche) and incubate at 4° C. on rotisserie for 2 hrs. Subsequent addition and incubation of the sample with 20 ul of Protein A/G plus agarose (Santa Cruz Biologics, 4° C. on rotisserie for 2 hrs) allows isolation of the Antibody-GFP-Perturbagen-Target complex by centrifugation (2500 RPM for 5 min at 4° C.). The pellet is then washed/centrifuged three times in IP-Wash Buffer (IP-buffer/PMSF/Protease inhibitors with 150 mM NaCl, 300 mM NaCl, or 450 mM NaCl) to remove non-specific/low-affinity binding contaminants. Following the final wash the pellet is then resuspend in 20 ul 2×sample loading buffer, boiled for 3-5 min spin and spun in a microcentrifuge to separate the pellet from the supernatant. The supernatant containing both the perturbagen and the target is then loaded on a SDS-polyacrylamide gel and visualized by silver stain. Each target is then identified by its molecular weight or alternative methods (e.g. mass spectrometry, peptide sequencing).

As is apparent to one of skill in the art, various modifications of the above embodiments can be made without departing from the spirit and scope of this invention. These modifications and variations are within the scope of this invention.

What is claimed is:

1. An isolated polypeptide having viral activity comprising a polypeptide sequence selected from the group consisting of:
(a) the polypeptide sequence of FIG. 11 (cW985);
(b) biologically active modifications of (a); and
(c) biologically active fragments of (a).

2. The isolated polypeptide of claim 1 wherein said polypeptide sequence is the polypeptide sequence of FIG. 11 (cW985).

3. The isolated polypeptide consisting essentially of the polypeptide sequence of FIG. 11 (cW985).

4. The isolated polypeptide of claim 3 wherein said isolated polypeptide comprises the polypeptide sequence of FIG. 11 (cW985) except for one or more conservative amino acid substitutions.

5. The isolated polypeptide consisting of the polypeptide sequence of FIG. 11 (cW985).

6. The isolated polypeptide of claim 1 wherein said isolated polypeptide comprises a sequence at least 99% identical to the polypeptide sequence of FIG. 11 (cW985).

7. The isolated polypeptide of claim 1 wherein said isolated polypeptide comprises a sequence at least 95% identical to the polypeptide sequence of FIG. 11 (cW985).

8. The isolated polypeptide of claim 1 wherein said isolated polypeptide comprises a sequence at least 90% identical to the polypeptide sequence of FIG. 11 (cW985).

9. The isolated polypeptide of claim 1 wherein said isolated polypeptide comprises a sequence at least 85% identical to the polypeptide sequence of FIG. 11 (cW985).

10. The isolated polypeptide of claim 1 wherein said isolated polypeptide comprises a sequence at least 80% identical to the polypeptide sequence of FIG. 11 (cW985).

11. The isolated polypeptide of claim 1 wherein said isolated polypeptide comprises a biologically active fragment of the polypeptide sequence of FIG. 11 (cW985) that displays viral activity.

12. The isolated polypeptide of claim 1 wherein said isolated polypeptide is a closely related analog of the polypeptide sequence of FIG. 11 (cW985), wherein said analog displays viral activity.

13. The isolated polypeptide of claim 1 wherein said isolated polypeptide is an antigenic analog of the polypeptide sequence of FIG. 11 (cW985), wherein said analog binds to an antibody specific for the polypeptide of FIG. 11 (cW985).

14. The isolated polypeptide of claim 1 wherein said isolated polypeptide is an N-terminal fragment of the polypeptide of FIG. 11 (cW985).

15. The isolated polypeptide of claim 14 wherein said N-terminal fragment comprises at least 10 amino acids of the polypeptide of FIG. 11 (cW985).

16. The isolated polypeptide of claim 1 wherein said isolated polypeptide is a C-terminal fragment of the polypeptide of FIG. 11 (cW985).

17. The isolated polypeptide of claim 16 wherein said C-terminal fragment comprises at least 10 amino acids of the polypeptide of FIG. 11 (cW985).

18. The polypeptide of claim 1 wherein said polypeptide is fused to heterologous sequence.

19. The polypeptide of claim 18 wherein said heterologous sequence is a scaffold.

20. The polypeptide of claim 19 wherein said scaffold is a fluorescent protein.

21. The polypeptide of claim 1 wherein said polypeptide is chemically modified.

22. The polypeptide of claim 21 wherein said polypeptide is radio labeled.

23. The polypeptide of claim 21 wherein said modification is selected from the group consisting of acetylation, glycosylation, or fluorescent tagging.

24. The isolated polypeptide of claim 1 wherein said polypeptide is chemically synthesized.

25. An isolated polynucleotide encoding a polypeptide of claim 1.

26. An isolated polynucleotide encoding a polypeptide of claim 1 wherein said polypeptide encodes the polypeptide sequence of FIG. 11 (cW985).

27. An isolated polynucleotide encoding a polypeptide of claim 3.

28. An isolated polynucleotide encoding a polypeptide of claim 4.

29. An isolated polynucleotide encoding a polypeptide of claim 5.

30. An isolated polynucleotide encoding a polypeptide of claim 6.

31. An isolated polynucleotide encoding a polypeptide of claim 7.

32. An isolated polynucleotide encoding a polypeptide of claim 8.

33. An isolated polynucleotide encoding a polypeptide of claim 9.

34. An isolated polynucleotide encoding a polypeptide of claim 10.

35. An isolated polynucleotide encoding a polypeptide of claim 14.

36. An isolated polynucleotide encoding a polypeptide of claim 16.

37. An isolated polynucleotide comprising the DNA sequence of FIG. 11 (cW985).

38. An isolated polynucleotide consisting essentially of the DNA sequence of FIG. 11 (cW985).

39. An isolated polynucleotide consisting of the DNA sequence of FIG. 11 (cW985).

40. The isolated polynucleotide of claim 37 wherein said isolated polynucleotide comprises a sequence at least 99% identical to said polynucleotide.

41. The isolated polynucleotide of claim 37 wherein said isolated polynucleotide comprises a sequence at least 95% identical to said polynucleotide.

42. The isolated polynucleotide of claim 37 wherein said isolated polynucleotide comprises a sequence at least 90% identical to said polynucleotide.

43. The isolated polynucleotide of claim 37 wherein said isolated polynucleotide comprises a sequence at least 85% identical to said polynucleotide.

44. The isolated polynucleotide of claim 37 wherein said isolated polynucleotide comprises a sequence at least 80% identical to said polynucleotide.

45. A vector comprising the polynucleotide of any one of claims 25, 26, 38 or 39.

46. The vector of claim 45, wherein said vector provides inducible expression.

47. A gene therapy vector comprising the polynucleotide of claims 25, 26, 38 or 39.

48. A host cell comprising the vector of claim 45.

49. A polynucleotide that hybridizes under stringent conditions to the polynucleotide of any one of claims 25, 26, 38 or 39.

50. A method for producing a polypeptide having viral activity comprising culturing a population of host cells of claim 48 under conditions suitable for the expression of an encoded polypeptide and recovering expressed polypeptide from the host cell culture.

51. A composition comprising the polypeptide of claims 1, 2, 3 or 5 in a pharmaceutically acceptable carrier.

52. An antibody to the polypeptide of claims 1, 2, 3 or 5.

53. A method of identifying a cellular target that interacts with a polypeptide having viral activity, comprising the steps of exposing a polypeptide of claim 1 to putative target molecules and identifying a polypeptide/target interaction pair.

54. The method of claim 53 wherein said method is a yeast two-hybrid assay.

55. A method of screening for putative viral related therapeutics, comprising the steps of:
a) exposing a polypeptide/target interaction pair obtained by the method of claim 53 to a plurality of agents; and
b) recovering a subpopulation of disrupting agents which competitively displace said polypeptide from said target; wherein said disrupting agents are putative viral related therapeutics.

56. The method of claim 55, wherein said plurality of agents is a combinatorial chemical library.

57. A method of treating a viral related condition, comprising the step of administering a therapeutically effective amount of the polypeptide of claim 1, or a pharmaceutically acceptable salt thereof.
